(19) United States
(12) Patent Application Publication
(10) Pub. No.: US 2004/0006081 A1
(43) Pub. Date: Jan. 8, 2004

(54) Pharmaceutically active piperidine derivatives, in particular as modulators of chemokine receptor activity

(76) Inventors: Jeremy Burrows, Macclesfield (GB); Anne Cooper, Loughborough (GB); John Cumming, Macclesfield (GB); Thomas McInally, Loughborough (GB); Howard Tucker, Macclesfield (GB)

Correspondence Address: Janis K Fraser, Fish & Richardson, 225 Franklin Street, Boston, MA, 02110-2804, US

(21) Appl. No.: 10/276,430

(22) PCT Filed: May 14, 2001
(86) PCT No.: PCT/SE01/01053

(51) Int. Cl. A61K 31/5377; A61K 31/506; A61K 31/503; A61K 31/501; A61K 31/496; A61K 31/4545; A61K 31/454; C07D 413/02; C 07D 4 3/02; C 07D 4 1/02

(52) U.S. Cl. 514/238.2; 514/248; 514/252.03; 514/256; 514/255.05; 514/310; 514/314; 514/253.01; 514/316; 514/318; 514/326; 544/129; 544/237; 544/238; 544/331; 544/360; 544/405; 546/148; 546/159; 546/186; 546/193; 546/207; 546/208; 546/210

(57) ABSTRACT

Compounds of formula (I), compositions comprising them, processes for preparing them and their use in medical therapy (for example modulating CCR5 receptor activity in a warm blooded animal).

The present invention relates to heterocyclic derivatives having pharmaceutical activity, to processes for preparing such derivatives, to pharmaceutical compositions comprising such derivatives and to the use of such derivatives as active therapeutic agents.

Pharmaceutically active piperidine derivatives are disclosed in EP-A1-1013276, WO00/08013, WO099/38514 and WO099/04794.

Chemokines are chemotactic cytokines that are released by a wide variety of cells to attract macrophages, T cells, eosinophils, basophils and neutrophils to sites of inflammation and also play a rôle in the maturation of cells of the immune system. Chemokines play an important role in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C-X-C, or α) and Cys-Cys (C-C, or β) families. These are distinguished on the basis of a single amino acid insertion between the NH-proximal pair of cysteine residues and sequence similarity.

The C-X-C chemokines include several potent chemoattractants and activators of neutrophils such as interleukin-8 (IL-8) and neutrophil-activating peptide 2 (NAP-2).

The C-C chemokines include potent chemoattractants of monocytes and lymphocytes but not neutrophils such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4. These receptors represent good targets for drug development since agents which modulate these receptors would be useful in the treatment of disorders and diseases such as those mentioned above.

The CCR5 receptor is expressed on T-lymphocytes, monocytes, macrophages, dendritic cells, microglia and other cell types. These detect and respond to several chemokines, principally “regulated on activation normal T-cell expressed and secreted” (RANTES), macrophage inflammatory proteins (MIP) MIP-1a and MIP-1b and monocyte chemoattractant protein-2 (MCP-2).

This results in the recruitment of cells of the immune system to sites of disease. In many diseases it is the cells expressing CCR5 which contribute, directly or indirectly, to tissue damage. Consequently, inhibiting the recruitment of these cells is beneficial in a wide range of diseases.

CCR5 is also a co-receptor for HIV-1 and other viruses, allowing these viruses to enter cells. Blocking the receptor with a CCR5 antagonist or inducing receptor internalisation with a CCR5 agonist protects cells from viral infection.

The present invention provides a compound of formula (I):

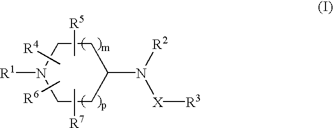

wherein:

R¹ is C₁₋₆ alkyl, C₃₋₇ cycloalkyl, C₃₋₈ alkenyl or C₃₋₈ alkynyl, each optionally substituted with one or more of: halo, hydroxy, cyano, nitro, C₃₋₇ cycloalkyl, NR⁸R⁹, C(O)R¹⁰, NR¹³C(O)R¹⁴, C(O)NR¹⁷R¹⁸, NR¹⁹C(O)NR²⁰R²¹, S(O)ₙR²², C₁₋₆ alkoxy (itself optionally substituted by heterocyclyl or C(O)NR²³R²⁴), heterocyclyl, heterocyclyloxy, aryl, aryloxy, heteroaryl or heteroaryloxy;

R² is hydrogen, C₁₋₈ alkyl, C₃₋₈ alkenyl, C₃₋₈ alkynyl, C₃₋₇ cycloalkyl, aryl, heteroaryl, heterocyclyl, aryl(C₁₋₄)alkyl, heteroaryl(C₁₋₄)alkyl or heterocyclyl(C₁₋₄)alkyl;

R³ is C₁₋₈ alkyl, C₂₋₈ alkenyl, NR⁴⁵R⁴⁶, C₂₋₈ alkynyl, C₃₋₇ cycloalkyl, C₃₋₇ cycloalkenyl, aryl, heteroaryl, heterocyclyl, aryl(C₁₋₄)alkyl, heteroaryl(C₁₋₄)alkyl or heterocyclyl(C₁₋₄)alkyl;

R⁴⁶ is C₁₋₈ alkyl, C₃₋₈ alkenyl, C₃₋₈ alkynyl, C₃₋₇ cycloalkyl, aryl, heteroaryl, heterocyclyl, aryl(C₁₋₄)alkyl, heteroaryl(C₁₋₄)alkyl or heterocyclyl(C₁₋₄)alkyl;

wherein the groups of R², R³ and R⁴⁶, and the heterocyclyl, aryl and heteroaryl moieties of R¹, are independently optionally substituted by one or more of halo, cyano, nitro, hydroxy, S(O)_{q}R²⁵, OC(O)NR²⁶R²⁷, NR²⁸R²⁹, NR³⁰C(O)R³¹, NR³²C(O)NR³³R³⁴, S(O)₂NR³⁵R³⁶, NR³⁷S(O)₂R³⁸, C(O)NR³⁹R⁴⁰, C(O)R⁴¹, CO₂R⁴², NR⁴³CO₂R⁴⁴, C₁₋₆ alkyl, C₃₋₁₀ cycloalkyl, C₁₋₆ haloalkyl, C₁₋₆ alkoxy, C₁₋₆ haloalkoxy, phenyl, phenyl(C₁₋₄)alkyl, phenoxy, phenylthio, phenyl(C₁₋₄)alkoxy, heteroaryl, heteroaryl(C₁₋₄)alkyl, heteroaryloxy or heteroaryl(C₁₋₄)alkoxy;

wherein any of the immediately foregoing phenyl and heteroaryl moieties are optionally substituted with halo, hydroxy, nitro, S(O)ₖC₁₋₄ alkyl, S(O)₂NH₂, cyano, C₁₋₄ alkyl, C₁₋₄alkoxy, C(O)NH₂, C(O)NH(C₁₋₄ alkyl), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; the C₃₋₇ cycloalkyl, aryl, heteroaryl and heterocyclyl moieties of R¹, R² and R³ being additionally optionally substituted with C₁₋₆ alkyl, C₂₋₆ alkenyl, C₂₋₆ alkynyl or C₁₋₆ alkoxy(C₁₋₆)alkyl;

R⁴, R⁵, R⁶ and R⁷ are, independently, hydrogen, C₁₋₆ alkyl {optionally substituted by halo, cyano, hydroxy, C₁₋₄ alkoxy, OCF₃, NH₂, NH(C₁₋₄ alkyl), N(C₁₋₄ alkyl)₂, NHC(O)(C₁₋₄ alkyl), N(C₁₋₄ alkyl)C(O)(C₁₋₄ alkyl), NHS(O)₂(C₁₋₄ alkyl), N(C₁₋₄ alkyl)S(O)₂(C₁₋₄ alkyl), CO₂(C₁₋₄ alkyl), C(O)NH(C₁₋₄ alkyl), C(O)N(C₁₋₄ alkyl)₂, C(O)NH₂, CO₂H, S(O)₂(C₁₋₄ alkyl), S(O)₂NH(C₁₋₄ alkyl), S(O)₂N(C₁₋₄ alkyl)₂, heterocyclyl or C(O)(heterocyclyl)}, S(O)₂NH₂, S(O)₂NH(C₁₋₄ alkyl), C(O)N(C₁₋₄ alkyl)₂, C(O)(C₁₋₄ alkyl), CO₂H, CO₂(C₁₋₄ alkyl) or C(O)(heterocyclyl); or two of R⁴, R⁵, R⁶ and R⁷ can join to form, together with the ring to which they are attached, a bicyclic ring system; or two of R⁴, R⁵, R⁶ and R⁷ can form an endocyclic bond (thereby resulting in an unsaturated ring system);

X is C(O), S(O)₂, C(O)C(O), a direct bond or C(O)C(O)NR⁴⁷;

k, m, n, p and q are, independently, 0, 1 or 2;

R²⁵, R²⁶, R²⁷, R²⁸, Re²⁹, R³⁰, R³¹, R³², R³³, R³⁴, R³⁵, R³⁶, R³⁷,

R³⁸, R³⁹, R⁴⁰, R⁴¹, Re⁴², R⁴³ and R⁴⁴ are, independently, C₁₋₈ alkyl, C₃₋₈ alkenyl, C₃₋₈ alkynyl, C₃₋₇ cycloalkyl, aryl, heteroaryl or heterocyclyl each or which is optionally substituted by halo, cyano, nitro, hydroxy, C₁₋₄ alkyl, C₁₋₄ alkoxy, SCH₃, S(O)CH₃, S(O)₂CH₃, NH₂, NHCH₃, N(CH₃)₂, NHC(O)NH₂, C(O)NH₂, NHC(O)CH₃, S(O)₂N(CH₃)₂, S(O)₂NHCH₃, CF₃, CHF₂, CH₂F, CH₂CF₃ or OCF₃; and R²⁶, R²⁷, R²⁸, R²⁹, R³⁰, R³¹, R³², R³³, R³⁴, R³⁵, R³⁶, R³⁷, R³⁹, R⁴⁰, R⁴¹, R⁴², R⁴³ and R⁴⁴ may additionally be hydrogen;

R⁸, R⁹, R¹⁰, R¹³, R¹⁴, R¹⁷, R¹⁸, R¹⁹, R²⁰, R²¹, R²³, R²⁴, R⁴⁵ and R⁴⁷ are, independently, hydrogen, alkyl {optionally substituted by halo, hydroxy, C₁₋₆ alkoxy, C₁₋₆ haloalkoxy, heterocyclyl or phenyl (itself optionally substituted by halo, hydroxy, cyano, C₁₋₄ alkyl or C₁₋₄ alkoxy)}, phenyl (itself optionally substituted by halo, hydroxy, nitro, S(O)ₖC₁₋₄ alkyl, S(O)₂NH₂, cyano, C₁₋₄ alkyl, C₁₋₄ alkoxy, C(O)NH₂, C(O)NH(C₁₋₄ alkyl), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃) or heteroaryl (itself optionally substituted by halo, hydroxy, nitro, S(O)ₖC₁₋₄ alkyl, S(O)₂NH₂, cyano, C₁₋₄ alkyl, C₁₋₄ alkoxy, C(O)NH₂, C(O)NH(C₁₋₄ alkyl), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃);

R²² is alkyl {optionally substituted by halo, hydroxy, C₁₋₆ alkoxy, C₁₋₆ haloalkoxy, heterocyclyl or phenyl (itself optionally substituted by halo, hydroxy, cyano, C₁₋₄ alkyl or C₁₋₄ alkoxy)}, phenyl (itself optionally substituted by halo, hydroxy, cyano, C₁₋₄ alkyl or C₁₋₄ alkoxy) or heteroaryl (itself optionally substituted by halo, hydroxy, cyano, C₁₋₄ alkyl or C₁₋₄ alkoxy);

the pairs of substituents: R⁸ and R⁹, R¹³ and R¹⁴, R¹⁷ and R¹⁸, R²⁰ and R²¹, R²³ and R²⁴, R²⁶ and R²⁷, R²⁸ and R²⁹, R³⁰ and R³¹, R³² with either R³³ or R³⁴, R³³ and R³⁴, R³⁵ and R³⁶, R³⁷ and R³⁸, R³⁹ and R⁴⁰ and R⁴³ and R⁴⁴ may, independently, join to form a ring and such a ring may also comprise an oxygen, sulphur or nitrogen atom;

where for any of the foregoing heterocyclic groups having a ring —N(H)— moiety, that —N(H)— moiety may be optionally substituted by C₁₋₄ alkyl (itself optionally substituted by hydroxy), C(O)(C₁₋₄ alkyl), C(O)NH(C₁₋₄ alkyl), C(O)N(C₁₋₄ alkyl)₂ or S(O)₂(C₁₋₄ alkyl);

a ring nitrogen and/or sulphur atom is optionally oxidised to form an N-oxide and/or an S-oxide;

foregoing heteroaryl or heterocyclyl rings are C- or, where possible, N-linked;

or a pharmaceutically acceptable salt thereof or a solvate thereof.

Certain compounds of the present invention can exist in different isomeric forms (such as enantiomers, diastereomers, geometric isomers or tautomers). The present invention covers all such isomers and mixtures thereof in all proportions.

Suitable salts include acid addition salts such as a hydrochloride, hydrobromide, phosphate, acetate, fumarate, maleate, tartrate, citrate, oxalate, methanesulphonate or p-toluenesulphonate.

The compounds of the invention may exist as solvates (such as hydrates) and the present invention covers all such solvates.

Alkyl groups and moieties are straight or branched chain and are, for example, methyl, ethyl, n-propyl or iso-propyl.

Alkenyl and alkynyl groups and moieties are, for example, vinyl, allyl or propargyl.

Cycloalkyl is a mono-, bi- or tri-cyclic structure such as, for example, cyclopropyl, cyclopentyl, cyclohexyl or adamantyl.

Cycloalkenyl comprises one double bond and is, for example, cyclopentenyl or cyclohexenyl.

Acyl is, for example, carbonyl substituted by either C₁₋₆ alkyl or optionally substituted phenyl.

Heterocyclyl is a non-aromatic 5 or 6 membered ring comprising at least one heteroatom selected from the group comprising nitrogen, oxygen and sulphur. Heterocyclyl is, for example, piperidinyl, morpholinyl, pyrrolidinyl, piperazinyl or tetrahydrofuryl.

Heteroaryl is an aromatic 5 or 6 membered ring comprising at least one heteroatom selected from the group comprising nitrogen, oxygen and sulphur. Heteroaryl is, for example, pyrrolyl, imidazolyl, pyrazolyl, 1,2,3-triazolyl, 1,2,4-triazolyl, oxazolyl, isoxazolyl, thiazolyl, isothiazolyl, pyridinyl, pyrimidinyl, pyrazinyl, pyridazinyl, thienyl, furyl, quinolinyl, isoquinolinyl, dihydroisoquinolinyl, indolyl, benzimidazolyl, benzo[b]furyl, benzo[b]thienyl, phthalazinyl, indanyl, oxadiazolyl or benzthiazolyl.

Aryl is a carbocyclic aromatic ring system (for example phenyl or naphthyl).

Arylalkyl is, for example, benzyl, 1-(phenyl)ethyl or 2-(phenyl)ethyl.

Heteroarylalkyl is, for example, pyridinylmethyl, pyrimidinylmethyl or 2-(pyridinyl)ethyl.

When R³⁹ and R⁴⁰ join to form a ring the ring is, for example, a piperazinyl, piperidinyl, pyrrolidinyl or morpholinyl ring.

In one aspect the invention provides a compound of formula (I) wherein X is C(O), S(O)₂ or a direct bond. In a further aspect X is C(O).

In another aspect the invention provides a compound of formula (I) wherein m and p are both 1.

In a further aspect the invention provides a compound of formula (I) wherein R⁴, R⁵, R⁶ and R⁷ are all hydrogen.

In yet another aspect the invention provides a compound of formula (I) wherein R² is hydrogen, C₁₋₄ alkyl (optionally substituted by C₃₋₆ cycloalkyl or phenyl), C₃₋₄ alkenyl or C₃₋₄ alkynyl. In another aspect R² is hydrogen.

In another aspect the invention provides a compound of formula (I) wherein R² is methyl, ethyl, allyl, cyclopropyl or propargyl.

In a further aspect the invention provides a compound of formula (I) wherein R² is methyl, ethyl or allyl.

In a still further aspect the invention provides a compound of formula (I) wherein R² is C₃₋₈ alkenyl (such as allyl) or C₃₋₇ cycloalkyl (such as cyclopropyl).

In a further aspect X is C(O).

In a still further aspect R³ is NR⁴⁵R⁴⁶, aryl, heteroaryl, aryl(C₁₋₄)alkyl or heteroaryl(C₁₋₄)alkyl; R⁴⁵ is hydrogen or C₁₋₆ alkyl; R⁴⁶ is aryl, heteroaryl, aryl(C₁₋₄)alkyl or heteroaryl(C₁₋₄)alkyl; wherein the aryl and heteroaryl groups of R³ and R⁴⁶ are independently substituted by S(O)_{q}R²⁵, OC(O)NR²⁶R²⁷, NR³²C(O)NR³³R³⁴ or C(O)R⁴¹, and optionally further substituted by one or more of halo, cyano, nitro, hydroxy, C₁₋₆ alkyl, C₂₋₆ alkenyl, C₂₋₆ alkynyl, C₁₋₆ alkoxy(C₁₋₆)alkyl, S(O)_{q}R²⁵, OC(O)NR²⁶R²⁷, NR²⁸R²⁹, NR³⁰C(O)R³¹, NR³²C(O)NR³³R³⁴, S(O)₂NR³⁵R³⁶, NR³⁷S(O)₂R³⁸, C(O)NR³⁹R⁴⁰, C(O)R⁴¹, CO₂R⁴², NR⁴³CO₂R⁴⁴, C₃₋₁₀ cycloalkyl, C₁₋₆ haloalkyl, C₁₋₆ alkoxy, C₁₋₆ haloalkoxy, phenyl, phenyl(C₁₋₄)alkyl, phenoxy, phenylthio, phenyl(C₁₋₄)alkoxy, heteroaryl, heteroaryl(C₁₋₄)alkyl, heteroaryloxy or heteroaryl(C₁₋₄)alkoxy;

wherein any of the immediately foregoing phenyl and heteroaryl moieties are optionally substituted with halo, hydroxy, nitro, S(O)ₖC₁₋₄ alkyl, S(O)₂NH₂, cyano, C₁₋₄ alkyl, C₁₋₄ alkoxy, C(O)NH₂, C(O)NH(C₁₋₄ alkyl), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; wherein q, ke, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³ and R⁴⁴ are as defined above.

In a still further aspect R³ is NR⁴⁵R⁴⁶, phenyl, heteroaryl, phenyl(C₁₋₄)alkyl or heteroaryl(C₁₋₄)alkyl; R⁴⁵ is hydrogen or C₁₋₆ alkyl; R⁴⁶ is phenyl, heteroaryl, phenyl(C₁₋₄)alkyl or heteroaryl(C₁₋₄)alkyl; wherein the phenyl and heteroaryl groups of R³ and R⁴⁶ are substituted by S(O)₂R²⁵, OC(O)NR²⁶R²⁷, NR³²C(O)NR³³R³⁴ or C(O)R⁴¹, and optionally further substituted by one or more of halo, cyano, nitro, hydroxy, C₁₋₆ alkyl, C₂₋₆ alkenyl, C₂₋₆ alkynyl, C₁₋₆ alkoxy(C₁₋₆)alkyl, S(O)₂R²⁵, OC(O)NR²⁶R²⁷, NR²⁸R²⁹, NR³⁰C(O)R³¹, NR³²C(O)NR³³R³⁴, S(O)₂NR³⁵R³⁶, NR³⁷S(O)₂R³⁸, C(O)NR³⁹R⁴⁰, C(O)R⁴¹, CO₂R⁴², NR CO₂R⁴³CO₂R⁴⁴, C₃₋₁₀ cycloalkyl, C₁₋₆ haloalkyl, C₁₋₆ alkoxy or C₁₋₆ haloalkoxy; wherein R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³ and R⁴⁴ are as defined above.

In another aspect R³ is NR⁴⁵R⁴⁶, phenyl, heteroaryl, phenyl(C₁₋₄)alkyl or heteroaryl(C₁₋₄)alkyl; R⁴⁵ is hydrogen or C₁₋₆ alkyl; R⁴⁶ is phenyl, heteroaryl, phenyl(C₁₋₄)alkyl or heteroaryl(C₁₋₄)alkyl; wherein the phenyl and heteroaryl groups of R³ and R⁴⁶ are substituted by S(O)₂R²⁵, and optionally further substituted by one or more of halo, cyano, nitro, hydroxy, C₁₋₆ alkyl, C₂₋₆ alkenyl, C₂₋₆ alkynyl, C₁₋₆ alkoxy(C₁₋₆)alkyl, C₁₋₆ haloalkyl, C₁₋₆ alkoxy or C₁₋₆ haloalkoxy; wherein R²⁵ is C₁₋₆ alkyl.

In yet another aspect R³ is NR⁴⁵R⁴⁶, phenyl or phenylCH₂; R⁴⁵ is hydrogen or C₁₋₂ alkyl; R⁴⁶ is phenyl or phenylCH₂; wherein the phenyl groups of R³ and R⁴⁶ are mono-substituted by S(O)₂R²⁵; wherein R²⁵ is C₁₋₆ alkyl (for example methyl).

In a further aspect R³ is phenyl or phenylCH₂; wherein the phenyl groups are mono-substituted (for example in the 4-position) by S(O)₂R²⁵; wherein R²⁵ is C₁₋₆ alkyl (for example methyl).

In another aspect R³ is NR⁴⁵R⁴⁶, phenyl, heteroaryl, phenyl(C₁₋₄)alkyl or heteroaryl(C₁₋₄)alkyl; R⁴⁵ is hydrogen or C₁₋₆ alkyl; R⁴⁶ is phenyl, heteroaryl, phenyl(C₁₋₄)alkyl or heteroaryl(C₁₋₄)alkyl; wherein the phenyl and heteroaryl groups of R³ and R⁴⁶ are substituted by S(O)₂NR³⁵R³⁶, and optionally further substituted byone or more of halo, cyano, nitro, hydroxy, C₁₋₆ alkyl, C₂₋₆ alkenyl, C₂₋₆ alkynyl, C₁₋₆ alkoxy(C₁₋₆)alkyl, C₁₋₆ haloalkyl, C₁₋₆ alkoxy or C₁₋₆ haloalkoxy; wherein R³⁵ and R³⁶ are, independently, hydrogen, C₁₋₈ alkyl, C₃₋₈ alkenyl, C₃₋₈ alkynyl, C₃₋₇ cycloalkyl, aryl, heteroaryl or heterocyclyl each or which is optionally substituted by halo, cyano, nitro, hydroxy, C₁₋₄ alkyl, C₁₋₄ alkoxy, SCH₃, S(O)CH₃, S(O)₂CH₃, NH₂, NHCH₃, N(CH₃)₂, NHC(O)NH₂, C(O)NH₂, NHC(O)CH₃, S(O)₂N(CH₃)₂, S(O)₂NHCH₃, CF₃, CHF₂, CH₂F, CH₂CF₃ or OCF₃.

In yet another aspect R³ is NR⁴⁵R⁴⁶, phenyl or phenylCH₂; R⁴⁵ is hydrogen or C₁₋₂ alkyl; R⁴⁶ is phenyl or phenylCH₂; wherein the phenyl groups of R³ and R⁴⁶ are mono-substituted by S(O)₂NR³⁵R³⁶; wherein R³⁵ and R³⁶ are, independently, hydrogen, C₁₋₈ alkyl, C₃₋₈ alkenyl, C₃₋₈ alkynyl, C₃₋₇ cycloalkyl, aryl, heteroaryl or heterocyclyl each or which is optionally substituted by halo, cyano, nitro, hydroxy, C₁₋₄ alkyl, C₁₋₄ alkoxy, SCH₃, S(O)CH₃, S(O)₂CH₃, NH₂, NHCH₃, N(CH₃)₂, NHC(O)NH₂, C(O)NH₂, NHC(O)CH₃, S(O)₂N(CH₃)₂, S(O)₂NHCH₃, CF₃, CHF₂, CH₂F, CH₂CF₃ or OCF₃; where, in a further aspect, R³⁵ is neither hydrogen nor C₁₋₄ alkyl.

In another aspect the present invention provides a compound of formula (I) wherein X is C(O); and R³ is C₃₋₇ cycloalkyl, (CH₂)₃-aryl, (CH₂)₃-heteroaryl, (CH₂)aryl, (CH₂)-heteroaryl, (CH₂)₃C(═O)NH-aryl, (CH₂)₃C(═O)NH-heteroaryl, (CH₂)C₃₋₁₀ cycloalkyl, (CH₂)₅NO₂, (CH₂)₅NC(═O)C₁₋₄ alkyl, CH₂—CH═CH-aryl, CH₂—CH═CH-heteroaryl, NH-aryl, NH-heterocyclyl, NH-allyl, NHCH₂-aryl or NHCH₂-heteroaryl; wherein aryl, heteroaryl and heterocyclyl groups are optionally substituted as defined above.

In a further aspect the present invention provides a compound of formula (I) wherein X is C(O); and R³ is (CH₂)₃-aryl, (CH₂)₃-heteroaryl, (CH₂)aryl, (CH₂)-heteroaryl, (CH₂)₃C(═O)NH-aryl, (CH₂)₃C(═O)NH-heteroaryl, NH-aryl, NH-heterocyclyl, NHCH₂-aryl or NHCH₂-heteroaryl; wherein aryl, heteroaryl and heterocyclyl rings are optionally substituted as defined above.

In a still further aspect the present invention provides a compound of formula (I) wherein X is C(O); and R³ is CH₂-phenyl (wherein the phenyl ring is optionally substituted at the 3-, 4- and/or 5-position with one or more substituents recited for aryl above), (CH₂)₃-phenyl, (CH₂)₃-oxadiazole-aryl, (CH₂)₃-oxadiazole-heteroaryl, (CH₂)₃C(═O)NH-phenyl, NHCH₂-phenyl, NHCH₂-heteroaryl or NH-phenyl (wherein the phenyl ring is optionally substituted at the 3-, 4- and/or 5-position with one or more substituents recited for aryl above); wherein aryl and heteroaryl rings are optionally substituted as defined above; phenyl rings are, unless stated otherwise, optionally substituted with one or more substituents recited for aryl above.

In yet another aspect the present invention provides a compound of formula (I) wherein X is C(O); and R³ is CH₂-phenyl [wherein the phenyl ring is optionally substituted at the 3-, 4- and/or 5-position with one or more of Cl, Br, F, OH, C₁₋₄ alkoxy (such as OMe or OEt), CN, S(O)₂(C₁₋₄ alkyl) (such as S(O)₂Me), S(O)(C₁₋₄ alkyl) (such as S(O)Me), S(C₁₋₄ alkyl) (such as SMe), S(O)₂NH₂, S(O)₂N(C₁₋₄ alkyl)₂ (such as S(O)₂NMe₂), C₁₋₄ alkyl (such as Me), CF₃, OCF₃, NO₂, NHC(O)(C₁₋₄ alkyl) (such as NHCOMe), C(O)(C₁₋₄ alkyl) (such as C(O)Me), S(O)₂CF₃, S(O)CF₃, SCF₃, C(O)NH₂ or CO₂(C₁₋₄ alkyl) (such as CO₂Me)], NHCH₂-phenyl [wherein the phenyl ring is optionally substituted at the 3-, 4- and/or 5-position with one or more of Cl, Br, F, OH, C₁₋₄ alkoxy (such as OMe or OEt), CN, S(O)₂(C₁₋₄ alkyl) (such as S(O)₂Me), S(O)(C₁₋₄ alkyl) (such as S(O)Me), S(C₁₋₄ alkyl) (such as SMe), S(O)₂NH₂, S(O)₂N(C₁₋₄ alkyl)₂ (such as S(O)₂NMe₂), CF₃, OCF₃, NO₂, NHC(O)(C₁₋₄ alkyl such as NHC(O)Me), C(O)(C₁₋₄ alkyl) (such as C(O)Me), S(O)₂CF₃, S(O)CF₃, SCF₃, C(O)NH₂ or CO₂(C₁₋₄ alkyl) (such as CO₂Me)] or NH-phenyl [wherein the phenyl ring is optionally substituted at the 3-, 4- and/or 5-position with one or more of F, Cl, C₁₋₄ alkoxy (such as OMe) or N(C₁₋₄ alkyl)₂ (such as NMe₂)].

In another aspect the present invention provides a compound of formula (I) wherein X is C(O); and R³ is CH₂-phenyl [wherein the phenyl ring is optionally substituted at the 4-position with Cl, Br, F, OH, OMe, CN, S(O)₂Me, S(O)₂NH₂, S(O)₂NMe₂, CF₃, OCF₃, NO₂, NHC(O)Me or CO₂Me], NHCH₂-phenyl [wherein the phenyl ring is optionally substituted at the 4-position with Cl, Me, F or OMe] or NH-phenyl [wherein the phenyl ring is optionally substituted at the 4-position with F, Cl, OMe or NMe₂).

In a further aspect the invention provides a compound as hereinbefore defined wherein R¹ is C₁₋₆ alkyl {optionally substituted by cyano, NR^{13*}C(O)R^{14*}, NR^{15*}R^{16*}, phenyl (itself optionally substituted by halo, hydroxy, nitro, S(O)ₖC₁₋₄ alkyl, S(O)₂NH₂, cyano, C₁₋₄ alkyl, C₁₋₄ alkoxy, C(O)NH₂, C(O)NH(C₁₋₄ alkyl), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃) or heteroaryl (itself optionally substituted by halo, hydroxy, nitro, S(O)ₖC₁₋₄ alkyl, S(O)₂NH₂, cyano, C₁₋₄ alkyl, C₁₋₄ alkoxy, C(O)NH₂, C(O)NH(C₁₋₄ alkyl), CO₂H, CO₂(C₁₋₄ alkyl), NHC(O)(C₁₋₄ alkyl), NHS(O)₂(C₁₋₄ alkyl), C(O)(C₁₋₄ alkyl), CF₃, OCF₃ or phenyl (itself optionally substituted by halo, hydroxy, nitro, S(O)ₖC₁₋₄ alkyl, S(O)₂NH₂, cyano, C₁₋₄ alkyl, C₁₋₄ alkoxy, C(O)NH₂, C(O)NH(C₁₋₄ alkyl), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃))} or C₂₋₆ alkenyl {optionally substituted by phenyl (itself optionally substituted by halogen, hydroxy, nitro, C₁₋₄ alkyl, C₁₋₄ alkoxy or di(C₁₋₄ alkyl)amino)}; R^{13*} is C₁₋₄ alkyl; R^{14*} is phenyl optionally substituted by halo, hydroxy, nitro, S(O)ₖC₁₋₄ alkyl, S(O)₂NH₂, cyano, C₁₋₄ alkyl, C₁₋₄ alkoxy, C(O)NH₂, C(O)NH(C₁₋₄ alkyl), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; and R^{15*} and R^{16*} are, independently, C₁₋₄ alkyl or phenyl (optionally substituted by halo, hydroxy, nitro, S(O)ₖC₁₋₄ alkyl, S(O)₂NH₂, cyano, C₁₋₄ alkyl, C₁₋₄ alkoxy, C(O)NH₂, C(O)NH(C₁₋₄ alkyl), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃). Heteroaryl is, for example, pyrrolyl, furyl, indolyl or pyrimidinyl.

In another aspect R¹ is a three-carbon chain which optionally carries one methyl group along its length (for example a methyl group is carried on the carbon that bonds to the nitrogen atom of the ring shown in formula (I)) wherein said three-carbon chain is optionally substituted as described for R¹ above.

In a still further aspect the invention provides a compound as hereinbefore defined wherein R¹ is 2,6-dimethoxybenzyl, 2,4,6-trimethoxybenzyl, 2,4-dimethoxy-6-hydroxybenzyl, 3-(4-dimethylamino-phenyl)prop-2-enyl, (1-phenyl-2,5-dimethylpyrrol-3-yl)methyl, 2-phenylethyl, 3-phenylpropyl, 3-R/S-phenylbutyl, 3-cyano-3,3-diphenylpropyl, 3-cyano-3-phenylpropyl, 4-(N-methylbenzamido)-3-phenylbutyl or 3,3-diphenylpropyl.

Further examples of R¹ include each individual partial structure presented in Schedule I and each individual partial structure presented in Schedule I can be combined with any definition of X, R², R³, R⁴, R⁵, R⁶, R⁷, m or p as herein defined.

In another aspect the invention provides a compound as hereinbefore defined wherein R¹ is 3-R/S-phenylbutyl or, preferably, 3,3-diphenylpropyl. In a further aspect R¹ is 3-(S)-phenylbutyl. In yet a further aspect R¹ is 3,3-diphenylpropyl.

In a still further aspect the present invention provides a compound of formula (I) wherein R¹ is a hereinbefore defined; R² is ethyl, allyl or cyclopropyl (for example allyl or cyclopropyl); and R³ is NHCH₂C₆H₅, NHCH₂(4-F—C₆H₄), NHCH₂(4-S(O)₂CH₃—C₆H₄), NHCH₂(4-S(O)₂NH₂—C₆H₄), CH₂C₆H₅, CH₂(4-F—C₆H₄), CH₂(4-S(O)₂CH₃—C₆H₄) or CH₂(4-S(O)₂NH₂—C₆H₄) {for example NHCH₂(4-S(O)₂CH₃—C₆H₄) or CH₂(4-S(O)₂CH₃—C₆H₄)}.

In yet another aspect the present invention provides a compound of formula (I) wherein R¹ is 3,3-diphenylpropyl, X is CO, R² is C₁₋₈ alkyl, and R³ is as hereinbefore defined.

In a further aspect the present invention provides a compound of formula (I) wherein R¹ is 3,3-diphenylpropyl, X is CO, R² is allyl, and R³ is as hereinbefore defined.

In a still further aspect the present invention provides a compound of formula (I) wherein R¹ is 3,3-diphenylpropyl or 3-R/S-phenylbutyl, X is C(O), R² is H, and R³ is as hereinbefore defined.

In another aspect the present invention provides a compound of formula (I) wherein R¹ is 3,3-diphenylpropyl or 3-R/S-phenylbutyl, X is C(O), R² is H or methyl, and R³ is NR⁴⁵R⁴⁶ (such as an amine group as hereinbefore defined for R³).

In yet another aspect the present invention provides a compound of formula (Ia):

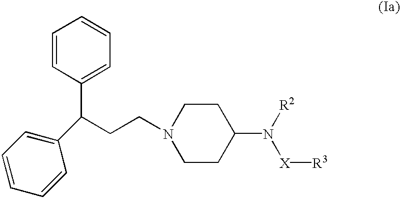

wherein X, R² and R³ are as defined above.

In a further aspect the present invention provides a compound of formula (Ib):

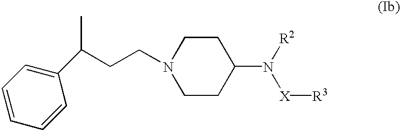

wherein X, R² and R³ are as defined above.

In a still further aspect the present invention provides a compound of formula (Ic):

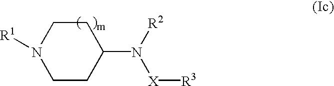

wherein X, m, R¹, R² and R³ are as defined above.

In yet another aspect the present invention provides a compound of formula (Id):

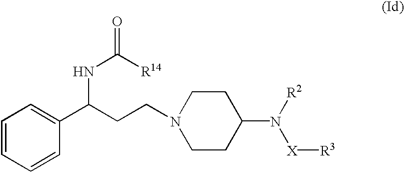

wherein X, R² and R³ are as defined above; and R¹⁴ is hydrogen, alkyl {optionally substituted by halo, hydroxy, C₁₋₆ alkoxy, C₁₋₆ haloalkoxy, heterocyclyl or phenyl (itself optionally substituted by halo, hydroxy, cyano, C₁₋₄ alkyl or C₁₋₄ alkoxy)}, phenyl (itself optionally substituted by halo, hydroxy, nitro, S(O)ₖC₁₋₄ alkyl, S(O)₂NH₂, cyano, C₁₋₄ alkyl, C₁₋₄ alkoxy, C(O)NH₂, C(O)NH(C₁₋₄ alkyl), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃), heteroaryl (itself optionally substituted by halo, hydroxy, nitro, S(O)ₖC₁₋₄ alkyl, S(O)₂NH₂, cyano, C₁₋₄ alkyl, C₁₋₄ alkoxy, C(O)NH₂, C(O)NH(C₁₋₄ alkyl), CO₂H, CO₂(C₁₋₄ alkyl), NHC(O)(C₁₋₄ alkyl), NHS(O)₂(C₁₋₄alkyl), C(O)(C₁₋₄ alkyl), CF₃ or OCF₃) or NR²⁰OR²¹; wherein R²⁰ and R²¹, together with the nitrogen to which they are attached, join to form an aziridine, azetidine or pyrrolidine ring.

The following compounds illustrate the invention.

TABLE I
Table I lists compounds of formula (Ia):

| | |
|---|---|
| 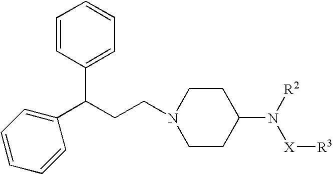 | (Ia) |

wherein X, R² and R³ are listed in the table. Mass Spectrum details are given for certain compounds of Table I.

TABLE II
Table II comprises 409 compounds of formula (Ib):

| | |
|---|---|
| | (Ib) |
| 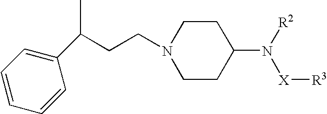 | |

| Compound | | | | LCMS |
|---|---|---|---|---|
| No. | X | R² | R³ | (MH+) |
| 1 | CO | Me | pyridin-4-yl | 415 |
| 2 | CO | Me | fur-3-yl | 404 |
| 3 | CO | Me | 4-(4-OH-C₆H_{4)C}₆H₄ | 506 |
| 4 | CO | Me | thien-3-yl | 419 |
| 5 | CO | Me | 2-NO₂-thien-4-yl | 464 |
| 6 | CO | Me | pyrazin-2-yl | 416 |
| 7 | CO | Me | 2,3-Cl₂-pyridin-5-yl | 482 |
| 8 | CO | Me | 2-Cl-6-Me-pyridin-4-yl | 462 |
| 9 | CO | Me | 3-Me-thien-2-yl | 434 |
| 10 | CO | Me | 3-Me-fur-2-yl | 418 |
| 11 | CO | Me | 2-CN-pyridin-5-yl | 440 |
| 12 | CO | Me | 2-NO₂-thiazol-4-yl | 477 |
| 13 | CO | Me | (CH₂)₅C₆H₅ | 483 |
| 14 | CO | Me | (CH₂)₂CONH(4-MeO-C₆H₄₎ | 514 |
| 15 | CO | Me | cyclopent-1-en-1-yl | 403 |
| 16 | CO | Me | (CH₂)₇COC₆H₅ | 540 |
| 17 | CO | Me | 4-tert-butyl-cyclohexyl | 476 |
| 18 | CO | Me | 2-Me-4,5,6,7-F₄-benzofur-3-yl | 539 |
| 19 | CO | Me | (CH₂)₃(3,4-(MeO)₂—C₆H₃₎ | 516 |
| 20 | CO | Me | (CH₂)₃CONH(C₆H₅) | 499 |
| 21 | CO | Me | (CH₂)_{2S(benzothiazol-2-yl)} | 530 |
| 22 | CO | Me | (CH₂)₃CONH(2-CN—C₆H₄) | 524 |
| 23 | CO | Me | CH₂(1-phenyl-5-methyl-iniidazol-4-yl) | 508 |
| 24 | CO | Me | CH₂(adamant-1-yl) | 486 |
| 25 | CO | Me | (CH₂)₃(1-Me-1,2-dihydro- | 537 |
| | | | isoquinolin-1-on-3-yl) | |
| 26 | CO | Me | CH₂(4-hydroxy-phthalazin-1-yl) | 496 |
| 27 | CO | Me | CH₂(1-Me-cyclohexyl) | 448 |
| 28 | Co | Me | CH₂(indan-2-yl) | 468 |
| 29 | Co | Me | 3-F-4-NO₂—C₆H₃ | 476 |
| 30 | Co | Me | CH₂NH(C₆H₅) | 443 |
| 31 | CO | Me | (CH₂)₅NO₂ | 453 |
| 32 | Co | Me | 2-Cl-pyridin-4-yl | 448 |
| 33 | CO | Me | (CH₂)₅NHCOCF₃ | 517 |
| 34 | CO | Me | CH₂(2-Me-3-NO₂—C₆H₃) | 486 |
| 35 | CO | Me | CH₂(3,5-(MeO)₂—C₆H₃) | 488 |
| 36 | CO | CH₂CH═CH₂ | CH₂(4-EtO—C₆H₄₎ | 497 |
| 37 | CO | CH₂CH═CH₂ | CH₂(5-F-indol-3-yl) | 510 |
| 38 | CO | CH₂CH═CH₂ | CH₂(3,4-(MeO)₂—C₆H₃) | 513 |
| 39 | CO | CH₂CH═CH₂ | CH₂(3,4,5-(MeO)₃—C₆H₂) | 543 |
| 40 | CO | CH₂CH═CH₂ | (CH₂)₃COC₆H₅ | 509 |
| 41 | CO | CH₂CH═CH₂ | CH₂(indol-3-yl) | 492 |
| 42 | CO | CH₂CH═CH₂ | CH₂(3,4-methylenedioxy-C₆H₃) | 497 |
| 43 | CO | CH₂CH═CH₂ | CH₂(4-I—C₆H₄) | 579 |
| 44 | CO | CH₂CH═CH₂ | CH₂(4-OCF₃—C₆H₄) | 537 |
| 45 | CO | CH₂CH═CH₂ | CH₂(3-Me-4-MeO—C₆H₃) | 497 |
| 46 | CO | CH₂CH═CH₂ | CH₂(3,4-(MeO)₂—C₆H₃) | 527 |
| 47 | CO | CH₂CH═CH₂ | CH₂(3-CF₃-4-F—C₆H₃) | 539 |
| 48 | CO | CH₂CH═CH₂ | CH₂(benzthien-3-yl) | 509 |
| 49 | CO | CH₂CH═CH₂ | (CH₂)₃(3-(pyridin-2-yl)-1,2,4- | 550 |
| | | | oxadiazol-5-yl) | |
| 50 | CO | CH₂CH═CH₂ | (CH₂)₃CO(thien-2-yl) | 515 |
| 51 | CO | CH₂CH═CH₂ | (CH₂)₃(4-Me—C₆H₄) | 495 |
| 52 | CO | CH₂CH═CH₂ | CH₂(5-MeO-indol-3-yl) | 522 |
| 53 | S(O)₂ | Me | 2-OCF₃—C₆H₄ | 533 |
| 54 | S(O)₂ | Me | 3-NO₂-4-Cl-C₆H₃ | 528 |
| 55 | S(O)₂ | Me | 2,5-Cl₂—C₆H₃ | 517 |
| 56 | S(O)₂ | Me | 2,5-Cl₂-thien-3-yl | 523 |
| 57 | S(O)₂ | Me | 2-Cl-5-CF₃—C₆H₃ | 551 |
| 58 | S(O)₂ | Me | 2-Cl-thien-2-yl | 489 |
| 59 | S(O)₂ | Me | 2-Cl-4-CF₃—C₆H₃ | 551 |
| 60 | S(O)₂ | Me | 2,4-F₂—C₆H₃ | 485 |
| 61 | S(O)₂ | Me | 2,3-Cl₂—C₆H₃ | 517 |
| 62 | S(O)₂ | Me | 2-NO₂—C₆H₄ | 494 |
| 63 | S(O)₂ | Me | 3-Cl-4-(NHCOMe)—C₆H₃ | 540 |
| 64 | S(O)₂ | Me | 2-CF₃—C₆H₄ | 517 |
| 65 | S(O)₂ | Me | 3,5-Me₂-isoxazol-4-yl | 468 |
| 66 | S(O)₂ | Me | 2-(isoxazol-3-yl)thien-5-yl | 522 |
| 67 | S(O)₂ | H | 3-Cl-4-(NHCOMe)—C₆H₃ | 526 |
| 68 | CO | Me | NH(3,4-Cl₂—C₆H₃) | 496 |
| 69 | CO | Me | NH(3-Cl-4-Me-C₆H₃) | 476 |
| 70 | CO | Me | NH(4-CF₃—C₆H₄) | 496 |
| 71 | CO | Me | NH(4-COMe—C₆H₄) | 471 |
| 72 | CO | Me | NH(2-Me-5-NO₂—C₆H₃) | 487 |
| 73 | CO | Me | NH(3,4-F₂—C₆H₃) | 464 |
| 74 | CO | Me | NH(CH₂)₂thien-2-yl | 462 |
| 75 | CO | Me | NH(4-I—C₆H₄) | 554 |
| 76 | CO | Me | NH(2-Et—C₆H₄) | 457 |
| 77 | CO | Me | NH(2,6-(Me)₂—C₆H₃) | 457 |
| 78 | CO | Me | NHCH₂(2,4-Cl₂—C₆H₃) | 510 |
| 79 | CO | H | NHCH₂C₆H₅ | 428 |
| 80 | CO | H | NH(4-Br—C₆H₄) | 494 |
| 81 | CO | H | NH(4-Cl—C₆H₄) | 448 |
| 82 | CO | H | NH(2-Cl—C₆H₄) | 448 |
| 83 | CO | H | NH(4-Me—C₆H₄) | 428 |
| 84 | CO | H | NH(2,6-Me₂-4-Br—C₆H₂) | 522 |
| 85 | CO | H | NH(2,4,6-Me₃—C₆H₂) | 456 |
| 86 | CO | H | NH(2-NO₂-4-Me—C₆H₃) | 473 |
| 87 | CO | H | NH(3-NO₂-4-Me—C₆H₃) | 473 |
| 88 | CO | H | NH(2-Me-3-NO₂—C₆H₃) | 473 |
| 89 | CO | H | NH(4-MeO—C₆H₄) | 444 |
| 90 | CO | H | NH(CH₂)2thien-2-yl | 448 |
| 91 | CO | H | NH-(n-propyl) | 380 |
| 92 | CO | H | NH(2,6-Me₂—C₆H₃) | 442 |
| 93 | CO | H | NH(2,6-F₂—C₆H₃) | 450 |
| 94 | CO | H | NH(4-NMe₂—C₆H₄) | 457 |
| 95 | CO | H | NHCH₂(2-Me—C₆H₄) | 442 |
| 96 | CO | Me | thien-2-yl | 419 |
| 97 | CO | Me | 2-NO₂-thien-5-yl | 448 |
| 98 | CO | Me | 3-NO₂—C₆H₄ | 458 |
| 99 | CO | Me | 4-NO₂—C₆H₄ | 458 |
| 100 | CO | Me | 4-F—C₆H₄ | 431 |
| 101 | CO | Me | 2-Cl-pyridin-5-yl | 448 |
| 102 | CO | Me | fur-2-yl | 403 |
| 103 | CO | Me | CH₂(4-Br—C₆H₄) | 507 |
| 104 | CO | Me | (CH₂)₂CO₂Me | 423 |
| 105 | CO | Me | cyclobutyl | 391 |
| 106 | CO | Me | (CH₂)₃(2-MeO—C₆H₄) | 471 |
| 107 | CO | Me | 1-(4-MeO—C₆H₄)cyclopropyl | 483 |
| 108 | CO | Me | (CH₂)₃indol-3-yl | 494 |
| 109 | COCO | Me | CH₂CH(CH₃)₂ | 421 |
| 110 | CO | Me | benzyl | 427 |
| 111 | CO | Me | CH₂(3,4-Cl₂—C₆H₃) | 495 |
| 112 | CO | Me | CH₂(tert-butyl) | 407 |
| 113 | CO | Me | CH₂(3,4,5-(MeO)₃—C₆H₂) | 517 |
| 114 | CO | Me | CH₂CH(CH₃)₂ | 393 |
| 115 | CO | Me | CH₂CH═CHC₆H₅ | 453 |
| 116 | CO | Me | CH₂CH₂SCH₃ | 411 |
| 117 | CO | Me | CH₂(4-Cl—C₆H₄) | 461 |
| 118 | CO | Me | 2,6-Cl₂-pyridin-3-yl | 482 |
| 119 | CO | Me | CH₂(2-F—C₆H₄) | 445 |
| 120 | CO | Me | CH₂(3-F—C₆H₄) | 445 |
| 121 | COCO | Me | phenyl | 441 |
| 122 | CO | Me | CH₂(2-Cl—C₆H₄) | 461 |
| 123 | CO | Me | CH₂(3-Cl—C₆H₄) | 461 |
| 124 | CO | Me | CH₂(3-MeO—C₆H₄) | 457 |
| 125 | CO | Me | CH₂(3,4-(MeO)₂—C₆H₃) | 487 |
| 126 | CO | Me | CH₂(4-F—C₆H₄) | 445 |
| 127 | CO | Me | CH₂(4-MeO—C₆H₄) | 457 |
| 128 | CO | Me | CH₂(2,4-F₂—C₆H₃) | 463 |
| 129 | CO | Me | CH₂(thien-2-yl) | 433 |
| 130 | CO | Me | CH₂(thien-3-yl) | 433 |
| 131 | CO | Me | CH₂(indol-3-yl) | 466 |
| 132 | CO | Me | CH₂(2,4-Cl₂—C₆H₃) | 495 |
| 133 | CO | Me | CH₂(3,4-F₂—C₆H₃) | 463 |
| 134 | CO | Me | CH₂(4-CF₃—C₆H₄) | 495 |
| 135 | CO | Me | CH₂(4-CF₃O-C₆H₄) | 511 |
| 136 | CO | Me | CHMe(C₆H₅) | 441 |
| 137 | CO | Me | CH₂(benzthien-3-yl) | 483 |
| 138 | CO | Me | CH₂(4-NO₂—C₆H₄) | 472 |
| 139 | CO | Me | (CH₂)₃(3-(pyridin-2-yl)-1,2,4- | 524 |
| | | | oxadiazol-5-yl) | |
| 140 | CO | H | CH₂(4-NO₂—C₆H₄) | 458 |
| 141 | CO | H | CH₂(3,4,5-(MeO)₃—C₆H₂) | 503 |
| 142 | CO | H | (CH₂)₃(3-(pyridin-2-yl)-1,2,4- | 510 |
| | | | oxadiazol-5-yl) | |
| 143 | CO | H | CH₂(4-Cl—C₆H₄) | 447 |
| 144 | CO | Me | NH(3-Cl—C₆H₄) | 462 |
| 145 | CO | Me | NHCH₂C₆H₅ | 442 |
| 146 | CO | Me | NH(cyclohexyl) | 434 |
| 147 | CO | Me | NH(phenyl) | 428 |
| 148 | CO | Me | NH(2-MeO—C₆H₄) | 458 |
| 149 | CO | Me | NH(3-Me—C₆H₄) | 442 |
| 150 | CO | Me | NH(4-Br—C₆H₄) | 508 |
| 151 | CO | Me | NH(4-Cl—C₆H₄) | 462 |
| 152 | CO | Me | NH(4-NO₂—C₆H₄) | 473 |
| 153 | CO | Me | NH(2-Br—C₆H₄) | 508 |
| 154 | CO | Me | NH(4-CO₂Et—C₆H₄) | 500 |
| 155 | CO | Me | NH(2-F—C₆H₄) | 446 |
| 156 | CO | Me | NH(2-Cl—C₆H₄) | 462 |
| 157 | CO | Me | NH(4-Me—C₆H₄) | 442 |
| 158 | CO | Me | NH(2,4,6-Me₃—C₆H₂) | 470 |
| 159 | CO | Me | NH(2-NO₂-4-Me—C₆H₃) | 487 |
| 160 | CO | Me | NH(2-Me-4-Cl—C₆H₃) | 476 |
| 161 | CO | Me | NH(3-CN—C₆H₄) | 453 |
| 162 | CO | Me | NH(3-NO₂-4-Me—C₆H₃) | 487 |
| 163 | CO | Me | NH(3-COMe—C₆H₄) | 470 |
| 164 | CO | Me | NH(3,5-Me₂—C₆H₃) | 456 |
| 165 | CO | Me | NH(2,4-Me₂—C₆H₃) | 456 |
| 166 | CO | Me | NH(2-Cl-4-NO₂—C₆H₃) | 507 |
| 167 | CO | Me | NH(2-Me-3-NO₂—C₆H₃) | 487 |
| 168 | CO | Me | NH(4-MeO—C₆H₄) | 458 |
| 169 | CO | Me | NH(n-propyl) | 394 |
| 170 | CO | Me | NHEt | 380 |
| 171 | CO | Me | NH(2-phenyl-cyclopropyl) | 468 |
| 172 | CO | Me | NH(CH₂CH═CH₂₎ | 392 |
| 173 | CO | Me | NH(naphth-2-yl) | 478 |
| 174 | CO | Me | NH(CH₂)₂C₆H₅ | 456 |
| 175 | CO | Me | NH(2,6-Cl₂-pyridin-4-yl) | 497 |
| 176 | CO | Me | NH(2,6-F₂—C₆H₃) | 464 |
| 177 | CO | Me | NH(4-N(Me)₂—C₆H₄) | 471 |
| 178 | CO | Me | NH(naphth-1-yl) | 478 |
| 179 | CO | Me | NH(2-Me—C₆H₄) | 442 |
| 180 | CO | Me | NH(2,6-Cl₂—C₆H₃) | 496 |
| 181 | CO | Me | NH(CH₂)₅CO₂Et | 494 |
| 182 | bond | Me | CH₂(4-Cl-imidazol-3-yl) | 424 |
| 183 | bond | Me | CH₂(2-(4-NO₂—C₆H₄)fur-5-yl) | 511 |
| 184 | bond | Me | CH₂(3-OH-4-NO₂—C₆H₃) | 461 |
| 185 | bond | Me | CH₂(4-Br-imidazol-3-yl) | 469 |
| 186 | bond | Me | CH₂(1-(4-Cl-benzyl)-imidazol-3-yl) | 514 |
| 187 | bond | H | CH₂(3-NO₂-4-OH—C₆H₃) | 447 |
| 188 | bond | H | CH₂(3-OH-4-NO₂—C₆H₃) | 447 |
| 189 | CO | Me | CH₂(2,2-Me₂-3-(COMe)-cyclobutyl) | |
| 190 | CO | Me | CH₂(3-MeO-4-OH-C₆H₃) | |
| 191 | CO | Me | CH₂(5-OH-indol-3-yl) | |
| 192 | CO | Me | CH₂(5-F-indol-3-yl) | |
| 193 | CO | Me | CH₂(4-OH—C₆H₄) | 443 |
| 194 | CO | CH₂C≡CH | (CH₂)₃cyclohexyl | |
| 195 | CO | CH₂C≡CH | CH₂CH₂CH(CH₃)C₆H₅ | |
| 196 | CO | CH₂CH═CH₂ | (CH₂)₃cyclohexyl | |
| 197 | CO | CH₂CH═CH₂ | CH₂(benzthien-3-yl) | |
| 198 | CO | CH₂CH═CH₂ | CH₂(4-(S(O)₂Me)—C₆H₄) | 536 |
| 199 | CO | CH₂cyclopropyl | (CH₂)₃cyclohexyl | |
| 200 | CO | (CH₂)₂phenyl | NH(2,4-F₂—C₆H₃) | |
| 201 | CO | H | NH(3,4-Cl₂—C₆H₃) | |
| 202 | CO | H | NH(2,4-Me₂—C₆H₃) | |
| 203 | CO | H | NH(2-Cl-4-NO₂—C₆H₃) | |
| 204 | CO | H | NH(4-MeO—C₆H₄) | |
| 205 | CO | H | NHCH₂(2,4-Cl₂—C₆H₃) | |
| 206 | CO | Me | CH₂(4-Me—C₆H₄) | 441 |
| 207 | CO | H | CH₂(3-Me—C₆H₄) | |
| 208 | CO | H | benzyl | |
| 209 | CO | H | CH₂(4-EtO—C₆H₄) | |
| 210 | CO | H | CH₂(3-F—C₆H₄) | |
| 211 | CO | H | CH₂(4-iso-propyl-C₆H₄) | |
| 212 | CO | H | CH₂-3-indole-5-OH | |
| 213 | CO | H | CH₂(4-Me—C₆H₄) | |
| 214 | CO | H | CH₂(3-Me-4-MeO—C₆H₃) | |
| 215 | CO | H | 5-F-indol-3-yl | |
| 216 | CO | H | CH₂(3,4-Cl₂—C₆H₃) | |
| 217 | CO | H | CH₂(4-phenyl-C₆H₄) | |
| 218 | CO | H | CH₂(3,4-F₂—C₆H₃) | |
| 219 | CO | H | CH₂(4-CF₃O—C₆H₄) | 497 |
| 220 | CO | H | CH₂(3-Br-4-MeO—C₆H₃) | |
| 221 | CO | H | CH₂(3-CF₃-4-F—C₆H₃) | |
| 222 | CO | H | CH₂(benzthien-3-yl) | |
| 223 | CO | H | CH₂(4-(S(O)₂NH₂)—C₆H₄) | |
| 224 | CO | H | CH₂(4-(S(O)₂NMe₂)—C₆H₄) | |
| 225 | CO | H | CH₂(3-CF₃—C₆H₄) | |
| 226 | CO | H | CH₂(3-Br—C₆H₄) | |
| 227 | CO | H | CH₂(4-Br—C₆H₄) | |
| 228 | CO | H | CH₂(4-(4-F—C₆H₄)—C₆H₄) | |
| 229 | CO | Me | NH(4-CF₃O—C₆H₄) | |
| 230 | CO | Me | NH(3-F—C₆H₄) | |
| 231 | CO | Me | NH(2,4-F₂—C₆H₃) | |
| 232 | CO | H | CH₂(4-NH₂—C₆H₄) | |
| 233 | CO | CH₂CH═CH₂ | CH₂(3,5-(MeO)₂-4-OH—C₆H₂) | 529 |
| 234 | CO | Me | CH₂(4-CN—C₆H₄) | 452 |
| 235 | CO | Me | CH₂(4-(S(O)₂NH₂)—C₆H₄) | 506 |
| 236 | CO | Me | CH₂(4-(S(O)₂NMe₂)—C₆H₄) | 534 |
| 237 | CO | H | CH₂(3,4-(OMe)₂—C₆H₃) | 473 |
| 238 | CO | H | CH₂(4-OMe—C₆H₄) | 443 |
| 239 | CO | H | CH₂(4-OH—C₆H₄) | 429 |
| 240 | CO | H | CH₂(4-CF₃—C₆H₄) | 481 |
| 241 | CO | H | CH₂(4-F—C₆H₄) | 431 |
| 242 | CO | H | CH₂(3-CF₃—C₆H₄) | |
| 243 | CO | CH₂CH═CH₂ | NH(4-F—C₆H₄) | 472 |
| 244 | CO | CH₂CH═CH₂ | NH(4-CH₃—C₆H₄) | 468 |
| 245 | CO | CH₂CH═CH₂ | NHCH₂C₆H₅ | 468 |
| 246 | CO | CH₂CH═CH₂ | NH(phenyl) | 454 |
| 247 | CO | CH₂CH═CH₂ | NH(4-OCH₃—C₆H₄) | 484 |
| 248 | CO | CH₂CH═CH₂ | NH((S)—CH₃CH(phenyl)) | 482 |
| 249 | CO | CH₂CH═CH₂ | NHCH₂CH═CH₂ | 418 |
| 250 | CO | CH₂CH═CH₂ | NHCH₂(3-CH₃—C₆H₄) | 482 |
| 251 | CO | CH₂CH═CH₂ | NHCH₂(4-OCH₃—C₆H₄) | 498 |
| 252 | CO | CH₂CH═CH₂ | NHCH₂(4-CH₃—C₆H₄) | 482 |
| 253 | CO | CH₂CH═CH₂ | NHCH₂(4-F—C₆H₄) | 486 |
| 254 | CO | Et | CH₂(4-F—C₆H₄) | 459 |
| 255 | CO | Et | CH₂(4-Cl—C₆H₄) | 475 |
| 256 | CO | Et | CH₂(4-NO₂—C₆H₄) | 486 |
| 257 | CO | Et | CH₂(4-CN—C₆H₄) | 466 |
| 258 | CO | Et | CH₂(4-S(O)₂NH₂—C₆H₄) | 520 |
| 259 | CO | Et | CH₂(4-S(O)₂N(CH₃)₂—C₆H₄) | 548 |
| 260 | CO | Et | NH(4-Me—C₆H₄) | 456 |
| 261 | CO | Et | NH(CHCH₃C₆H₅) | 470 |
| 262 | CO | Et | NHCH₂CH═CH₂ | 406 |
| 263 | CO | Et | NHCH₂C₆H₅ | 456 |
| 264 | CO | Et | NHCH₂(3-Me—C₆H₄) | 470 |
| 265 | CO | Et | NHCH₂(4-OMe—C₆H₄) | 486 |
| 266 | CO | Et | NHCH₂(4-Me—C₆H₄) | 470 |
| 267 | CO | Et | NHCH₂(4-F—C₆H₄) | 474 |
| 268 | CO | Me | CH₂(4-(OCH₂C₆H₄)—C₆H₄) | 533 |
| 269 | CO | CH₂CH═CH₂ | CH₂(3-F—C₆H₄) | 471 |
| 270 | CO | CH₂CH═CH₂ | (CH₂)₃-3-(4-Cl—C₆H₄)— | 583 |
| | | | [1,2,4]oxadiazol-5-yl | (585) |
| 271 | CO | CH₂CH═CH₂ | (CH₂)₃-3-(3-NO₂—C₆H₄)— | 594 |
| | | | [1,2,4]oxadiazol-5-yl | |
| 272 | CO | CH₂CH═CH₂ | CH₂(3-OMe—C₆H₄) | 483 |
| 273 | CO | CH₂CH═CH₂ | CH₂(4-Br—C₆H₄) | 533/531 |
| 274 | CO | CH₂CH═CH₂ | CH₂(4-Cl—C₆H₄) | 487 |
| | | | | (489) |
| 275 | CO | CH₂CH═CH₂ | CH₂(4-OMe—C₆H₄) | 483 |
| 276 | CO | CH₂CH═CH₂ | CH₂(4-CF₃—C₆H₄) | 521 |
| 277 | CO | Me | CH₂(4-NHC(O)Me—C₆H₄) | 484 |
| 278 | CO | Me | CH₂(4-SMe—C₆H₄) | 473 |
| 279 | CO | Me | CH₂(4-CO₂Me—C₆H₄) | 485 |
| 280 | CO | CH₂CH═CH₂ | CH₂(3,5-(OMe)₂-4-OH—C₆H₄ | 529 |
| 281 | CO | Me | CH₂(4-S(O)₂Me—C₆H₄) | 505 |
| 282 | CO | Et | CH₂(4-OCF₃—C₆H₄) | 525 |
| 283 | CO | Et | CH₂(4-S(O)₂Me—C₆H₄) | 519 |
| 284 | CO | cPr | CH₂(4-NO₂—C₆H₄) | 498 |
| 285 | CO | cPr | CH₂(4-OCF₃—C₆H₄) | 537 |
| 286 | CO | cPr | CH₂(4-S(O)₂Me—C₆H₄) | 531 |
| 287 | CO | cPr | CH₂(4-S(O)₂NH₂—C₆H₄) | 532 |
| 288 | CO | cPr | CH₂(4-F—C₆H₄) | 471 |
| 289 | CO | (CH₂)₂OH | CH₂(4-NO₂—C₆H₄) | 502 |
| 290 | CO | (CH₂)₂OH | CH₂(4-OCF₃C₆H₄) | 541 |
| 291 | CO | (CH₂)₂OH | CH₂(4-S(O)₂Me—C₆H₄) | 535 |
| 292 | CO | (CH₂)₂OH | CH₂(4-S(O)₂NH₂—C₆H₄) | 536 |
| 293 | CO | (CH₂)₂OH | CH₂(4-F—C₆H₄) | 475 |
| 294 | CO | (CH₂)₂F | CH₂(4-NO₂—C₆H₄) | 504 |
| 295 | CO | (CH₂)₂F | CH₂(4-OCF₃—C₆H₄) | 543 |
| 296 | CO | (CH₂)₂F | CH₂(4-S(O)₂Me—C₆H₄) | 537 |
| 297 | CO | (CH₂)₂F | CH₂(4-S(O)₂NH₂—C₆H₄) | 538 |
| 298 | CO | (CH₂)₂F | CH₂(4-F—C₆H₄) | 477 |
| 299 | CO | CH₂CH═CH₂ | CH₂(4-NO₂—C₆H₄) | 498 |
| 300 | CO | CH₂CH═CH₂ | CH₂(4-S(O)₂NH₂—C₆H₄) | 532 |
| 301 | CO | CH₂CH═CH₂ | CH₂(4-F—C₆H₄) | 471 |
| 302 | CO | cPr | CH₂(pyridin-2-yl) | 454 |
| 303 | CO | cPr | CH₂(1-Me-imidazol-4-yl) | 457 |
| 304 | CO | cPr | CH₂(1-Me-4-NO₂-pyrazo1-5-yl) | 502 |
| 305 | CO | cPr | CH₂(6-Cl-pyridin-3-yl) | 488 |
| | | | | (490) |
| 306 | CO | cPr | CH₂(3-Me-isoxazol-5-yl) | 458 |
| 307 | CO | cPr | CH₂(3,5-Me₂-isoxazol-4-yl) 472 | |
| 308 | CO | Et | CH₂(5-Cl-thien-2-yl) | 481 |
| | | | | (483) |
| 309 | CO | Et | CH₂(5-(NHCO₂-tert-Bu)- | 564 |
| | | | [2,4]oxadiazol-3-yl) | |
| 310 | CO | Et | CH₂(6-Cl-pyridin-3-yl) | 476 |
| | | | | (478) |
| 311 | CO | Et | CH₂(3,5-Me₂-isoxazol-4-yl) | 460 |
| 312 | CO | Et | CH₂(3-Me-isoxazol-5-yl) | 446 |
| 313 | CO | Et | CH₂(1-Me-4-NO₂-pyrazol-5-yl) | 490 |
| 314 | CO | (CH₂)₂phenyl | NH(2,4-F₂—C₆H₃) | 555 |
| 315 | CO | H | NH(2,4-Me₂—C₆H₃) | 422 |
| 316 | CO | cPr | NHCH₂C₆H₅ | 468 |
| 317 | CO | (CH₂)₂OCONHCH₂phenyl | NHCH₂C₆H₅ | 605 |
| 318 | CO | (CH₂)₂OH | NHCH₂C₆H₅ | 472 |
| 319 | CO | (CH₂)₂F | NHCH₂C₆H₅ | 474 |
| 320 | CO | cPr | NHCH₂(4-F—C₆H₄) | 486 |
| 321 | CO | (CH₂)₂OH | NHCH₂(4-F—C₆H₄) | 490 |
| 322 | CO | (CH₂)₂F | NHCH₂(4-F—C₆H₄) | 492 |
| 323 | CO | Et | NHCH₂(4-CF₃—C₆H₄) | 524 |
| 324 | CO | Et | NHCH₂(thien-3-yl) | 462 |
| 325 | CO | Et | NHCH₂(indol-3-yl) | 495 |
| 326 | CO | Et | NHCH₂(5-OMe-indol-3-yl) | 525 |
| 327 | CO | Et | NHCH₂(2,5-F₂—C₆H₃) | 492 |
| 328 | CO | Et | NHCH₂(3-Cl-4-OH—C₆H₃) | 507 |
| 329 | CO | Et | NHCH₂(thien2-yl) | 462 |
| 330 | CO | Et | NHCH₂(3-OMe—C₆H₄) | 486 |
| 331 | CO | Et | NHCH₂(2,6-F₂—C₆H₃) | 492 |
| 332 | CO | Et | NHCH₂(3,5-F₂—C₆H₃) | 492 |
| 333 | CO | Et | NHCH₂(2-F—C₆H₄) | 474 |
| 334 | CO | Et | NHCH₂(4-OCF₃—C₆H₄) | 540 |
| 335 | CO | Et | NHCH₂(2,2-Me₂-3-C(O)Me—cBu) | 504 |
| 336 | CO | Et | NHCH₂(2-phenyl-5-Me-oxazol-4-yl) | 537 |
| 337 | CO | Et | NH(indazol-3-yl) | 482 |
| 338 | CO | Et | NHCH₂(4-S(O)₂Me—C₆H₄) | 534 |
| 339 | CO | Et | NHCH₂(2-OMe—C₆H₄) | 486 |
| 340 | CO | Et | NHCH₂(3,5-Me₂-isoxazol-4-yl) | 475 |
| 341 | CO | Et | NHCH₂(5-phenyl-[1,2,4]triazol-3-yl) | 523 |
| 342 | CO | Et | NHCH₂(5-CN-indol-3-yl) | 520 |
| 343 | CO | Et | NHCH₂(2,5-(OMe)₂—C₆H₃) | 516 |
| 344 | CO | Et | NHCH₂(3-F—C₆H₄) | 474 |
| 345 | CO | Et | NLICH₂(3,4-(OMe)₂—C₆H₃) | 516 |
| 346 | CO | Et | NHCH₂(3,4,5-(OMe)₃—C₆H₄ | 546 |
| 347 | CO | Et | NHCH₂(3-OH—C₆H₄) | 472 |
| 348 | CO | Et | NHCH₂(4-OH—C₆H₄) | 472 |
| 349 | CO | Et | NHCH₂-(3-F-4-OH—C₆H₃) | 490 |
| 350 | CO | Et | NHCH₂(3-OMe-4-OH—C₆H₃) | 502 |
| 351 | CO | Et | NHCH₂(4-NH₂—C₆H₄) | 471 |
| 352 | CO | Et | NHCH₂(3,5-(OMe)₂-4-OH—C₆H₂) | 532 |
| 353 | CO | Et | NHCH₂(3-NH₂—C₆H₄) | 471 |
| 354 | CO | Me | CH₂(4-(S(O)₂NH—cPr)—C₆H₄) | 546 |
| 355 | CO | Me | CH₂(4-(S(O)₂NH-isoBu)-C₆H₄) | 562 |
| 356 | CO | Me | CH₂(4-(S(O)₂NH(CH₂)₂OMe)—C₆H₄) | 564 |
| 357 | CO | Me | CH₂(4-(S(O)₂NH(CH₂)₂OH)—C₆H₄) | 550 |
| 358 | CO | Me | CH₂(4-(S(O)₂NHCH₂C≡CH)—C₆H₄) | 544 |
| 359 | CO | Me | CH₃(4-(S(O)₂NHCH₂CH═CH_{2)—} | 546 |
| | | | C₆H₄) | |
| 360 | CO | Me | CH₂(4-(S(O)₂NH(CH₂)₃OH)—C₆H₄) | 564 |
| 361 | CO | Me | CH₂(4-(S(O)₂N(Me)CH₂C≡CH)— | 558 |
| | | | C₆H₄) | |
| 362 | CO | Me | CH₂(4-(S(O)₂N(Me)CH₂CH═CH_{2)—} | 560 |
| | | | C₆H₄) | |
| 363 | CO | Me | CH₂(4-(S(O)₂N(Me)Et)—C₆H₄ | 548 |
| 364 | CO | Me | CH₂-4-(S(O)₂N(Me)(CH₂)₂OH)— | 564 |
| | | | C₆H₄) | |
| 365 | CO | Me | CH₂(4-(S(O)₂NHCH₂—cPr)—C₆H₄) | 560 |
| 366 | CO | Me | CH₂(4-(S(O)₂N(Me)isoPr)—C₆H₄) | 562 |
| 367 | CO | Me | CH₂(4-(S(O)₂NHCH(Me)CH₂OH)— | 564 |
| | | | C₆H₄) | |
| 368 | CO | Me | CH₂(4-(S(O)₂-azetidinyl)-C₆H₄) | 546 |
| 369 | CO | Me | CH₂(4-(S(O)₂-pyrrolidinyl)-C₆H₄) | 560 |
| 370 | CO | Me | CH₂(4-(S(O)₂-morpholin-4-yl)-C₆H₄) | 576 |
| 371 | CO | Me | CH₂(4-(S(O)₂NH-isoPr)—C₆H₄) | 548 |
| 372 | CO | Me | CH₂(4-(S(O)₂NHMe)—C₆H₄) | 520 |
| 373 | CO | Me | CH₂(4-(S(O)₂NHCH₂CH(Me)OH)— | 564 |
| | | | C₆H₄) | |
| 374 | CO | Me | CH₂(4-(S(O)₂-3-CH₂OH-piperidin-1- | 604 |
| | | | yl)-C₆H₄) | |
| 375 | CO | Me | CH₂(4-(S(O)₂NH(CH₂)₂-imidazol-4- | 600 |
| | | | yl)-C₆H₄) | |
| 376 | CO | Me | CH₂(4-(S(O)₂-3-CH₂OH-pyrrolidin- | 590 |
| | | | 1-yl)-C₆H₄) | |
| 377 | CO | Me | CH₂(4-(S(O)₂-3-OH-piperidin-1-yl- | 590 |
| | | | C₆H₄) | |
| 379 | CO | Me | CH₂(4-(S(O)₂NH-pyridin-3-yl)-C₆H₄) | 583 |
| 380 | CO | Me | CH₂(4-(S(O)₂NHCH₂CN)—C₆H₄) | 545 |
| 381 | CO | Me | CH₂(4-(S(O)₂-pyrrolen-1-yl)-C₆H₄) | 558 |
| 382 | CO | Me | CH₂(4-(S(O)₂-4-OH-piperidin-1-yl)- | 590 |
| | | | C₆H₄) | |
| 383 | CO | Me | CH₂(4-(S(O)₂NH-pyrazo1-3yl)-C₆H₄) | 572 |
| 384 | CO | Me | CH₂(4-(S(O)₂-3-OH-pyrrolidin-1-yl)- | 576 |
| | | | C₆H₄) | |
| 385 | CO | Me | CH₂(4-(S(O)₂NH(CH₂)₂OH)—C₆H₄) | 514 |
| 386 | CO | Me | CH₂(4-(S(O)₂NH(CH₂)₃OH)—C₆H₄) | 528 |
| 387 | CO | Me | CH₂(4-(S(O)₂NHCH₂CH(OH)Me)— | 528 |
| | | | C₆H₄) | |
| 388 | CO | Me | NH(4-F—C₆H₄) | 446 |
| 389 | CO | Me | NHCH(Me)phenyl | 456 |
| 390 | CO | H | CH(CH₂CH═CH₂)-4-S(O)₂Me—C₆H₄ | 531 |
| 391 | CO | Me | pyrrolidin-1yl | 406 |
| 392 | CO | H | CH₂(1,3-benzodioxol-5-yl) | 395 |
| 393 | CO | H | CH₂(4-NMe₂—C₆H₄) | 394 |
| 394 | CO | H | CH₂(3-Cl-4-OH—C₆H₃) | 402 |
| | | | | (404) |
| 395 | CO | H | CH₂(4-CO₂Me—C₆H₄) | 409 |
| 396 | CO | H | CH₂(3-CN-4-OH—C₆H₃) | 392 |
| 397 | CO | H | CH₂(3-F-4-(thiomorphlin-4-yl)-C₆H₃) | 470 |
| 398 | CO | H | CH₂(3-OMe—C₆H₄) | 381 |
| 399 | CO | H | CH₂(3-OH—C₆H₄) | 367 |
| 400 | CO | H | CH₂(3-F-4-OH—C₆H₃) | 384 |
| 401 | CO | Et | NHCH₂(4-S(O)₂Me—C₆H₄) | |
| 402 | CO | Et | NHCH₂(4-S(O)₂NH₂—C₆H₄) | |
| 403 | CO | Et | CH₂C₆H₅ | |
| 404 | CO | CH₂CH═CH₂ | NHCH₂(4-S(O)₂Me—C₆H₄) | |
| 405 | CO | CH₂CH═CH₂ | NHCH₂(4-S(O)₂NH₂—C₆H₄) | |
| 406 | CO | CH₂CH═CH₂ | CH₂C₆H₅ | |
| 407 | CO | cPr | NHCH₂(4-S(O)₂Me—C₆H₄) | |
| 408 | CO | cPr | NHCH₂(4-S(O)₂NH₂—C₆H₄) | |
| 409 | CO | cPr | CH₂C₆H₅ | |

wherein the variables X, R² and R³ for each compound of Table II are the same as the correspondingly numbered compound in Table I. Mass Spectrum details are given for certain compounds of Table II.

| Example | MS |
|---|---|
| Number | (MH+) |
| 38 | 451 |
| 71 | 408 |
| 79 | 366 |
| 80 | 430 |
| 81 | 386 |
| 83 | 366 |
| 86 | 411 |
| 88 | 411 |
| 103 | 445 |
| 107 | 421 |
| 108 | 432 |
| 110 | 365 |
| 111 | 433 |
| 112 | 345 |
| 115 | 391 |
| 117 | 399 |
| 118 | 433 |
| 122 | 399 |
| 123 | 399 |
| 126 | 383 |
| 127 | 395 |
| 128 | 401 |
| 129 | 371 |
| 130 | 371 |
| 131 | 404 |
| 132 | 433 |
| 133 | 401 |
| 134 | 433 |
| 135 | 449 |
| 140 | 396 |
| 140 (R) | 396 |
| 140 (S) | 396 |
| 143 (R) | 385 (387) |
| 143 (S) | 385 (387) |
| 144 | 400 |
| 145 | 380 |
| 147 | 366 |
| 150 | 444 |
| 151 | 400 |
| 157 | 380 |
| 160 | 414 |
| 165 | 394 |
| 166 | 445 |
| 168 | 396 |
| 189 | 414 |
| 190 | 411 |
| 191 | 420 |
| 192 | 422 |
| 193 | 381 |
| 194 | 423 |
| 195 | 467 |
| 196 | 425 |
| 197 | 447 |
| 198 | 469 |
| 199 | 439 |
| 200 | 492 |
| 201 | 420 |
| 202 | 380 |
| 203 | 431 |
| 204 | 382 |
| 205 | 434 |
| 206 | 379 |
| 207 | 365 |
| 208 | 351 |
| 209 | 395 |
| 210 | 369 |
| 211 | 393 |
| 212 | 406 |
| 213 | 365 |
| 214 | 395 |
| 215 | 408 |
| 216 | 419 |
| 217 | 427 |
| 218 | 387 |
| 219 | 435 |
| 220 | 461 |
| 221 | 437 |
| 222 | 407 |
| 223 | 430 |
| 224 | 458 |
| 225 | 419 |
| 226 | 431 |
| 227 | 429 (431) |
| 228 | 445 |
| 229 | 450 |
| 230 | 383 |
| 231 | 402 |
| 232 | 366 |
| 237 | 411 |
| 239 | 367 |
| 240 | 419 |
| 245 | 406 |
| 392 | 395 |
| 393 | 394 |
| 394 | 402 (404) |
| 395 | 409 |
| 396 | 392 |
| 397 | 470 |
| 398 | 381 |
| 399 | 367 |
| 400 | 384 |

TABLE IlI
Table III discloses compounds of formula (Ic):

| | |
|---|---|
| | (Ic) |
| 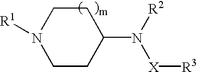 | |

wherein the variables R¹, X, R² and R³ are as defined in the Table below.
Mass Spectrum details are given for certain compounds of Table III.

| Compound No. | R¹ | m | X | R² | R³ | LCMS (MH+) |
|---|---|---|---|---|---|---|
| 1 | CH₂(2,6-(MeO)₂—C₆H₃) | 1 | CO | (CH₂)₂phenyl | NH(2,4-F₂—C₆H₃) | 510 |
| 2 | CH₂(2-(4-NO₂—C₆H₄)-fur-5-yl) | 1 | CO | (CH₂)₂phenyl | NH(2,4-F₂—C₆H₃) | 561 |
| 3 | CH₂(3-OH-4-NO₂—C₆H₃) | 1 | CO | (CH₂)₂phenyl | NH(2,4-F₂—C₆H₃) | 511 |
| 4 | CH₂(2-Et-fur-5-yl) | 1 | CO | (CH₂)₂phenyl | NH(2,4-F₂—C₆H₃) | 468 |
| 5 | CH₂(3-Me—C₆H₄) | 1 | CO | (CH₂)₂phenyl | NH(2,4-F₂—C₆H₃) | 463 |
| 6 | CH₂(2,4-MeO₂-pyrimidin-5-yl) | 1 | CO | (CH₂)₂phenyl | NH(2,4-F₂—C₆H₃) | 512 |
| 7 | CH₂(indol-3-yl) | 1 | CO | (CH₂)₂phenyl | NH(2,4-F₂—C₆H₃) | 489 |
| 8 | CH₂(1-phenyl-pyrrol-3-yl) | 1 | CO | (CH₂)₂phenyl | NH(2,4-F₂—C₆H₃) | 515 |
| 9 | (CH₂)₃phenyl | 1 | CO | (CH₂)₂phenyl | NH(2,4-F₂—C₆H₃) | 464 |
| 10‡ | iso-propyl | 1 | CO | 4-Cl—C₆H₄ | benzyl | |
| 11 | (CH₂)₂C(C₆H₅)(4-F—C₆H₄)OH | 1 | CO | Et | CH₂(4-S(O)₂Me—C₆H₄) | 553 |
| 12 | (CH₂)₂CH(CH═CH₂)C₆H₅ | 1 | CO | Me | CH₂(4-F—C₆H₄) | 395 |
| 13 | (CH₂)₂CH(C₆H₅)azetidin-1-yl | 1 | CO | Me | CH₂(4-F—C₆H₄) | 424 |
| 14 | (CH₂)₂CH(C₆H₅)pyrrolidin-1-yl | 1 | CO | Me | CH₂(4-F—C₆H₄) | 438 |
| 15 | (CH₂)₂CH(C₆H₅)(4-F—C₆H₄) | 1 | CO | Me | CH₂(4-F—C₆H₄) | 463 |
| 16 | (CH₂)₂CH(4-F—C₆H₄)₂ | 1 | CO | Me | CH₂(4-F—C₆H₄) | 481 |
| 17 | (CH₂)₂CH(4-F—C₆H₄)₂ | 1 | CO | Me | CH₂(4-S(O)₂NH₂—C₆H₄) | 542 |
| 18 | (CH₂)₂N(C₆H₅)₂ | 1 | CO | CH₂CH═CH₂ | CH₂(4-S(O)₂Me—C₆H₄) | 532 |
| 19 | (CH₂)₂N(C₆H₅)₂ | 1 | CO | Me | CH₂(4-F—C₆H₄) | 446 |
| 20 | (CH₂)₂N(C₆H₅)CO(CH₂)₂(4-OH— | 1 | CO | Et | CH₂(4-S(O)₂Me—C₆H₄) | 591 |
| | C₆H₄) | | | | | |
| 21 | (CH₂)₂N(C₆H₅)CO(2-SMe- | 1 | CO | Et | CH₂(4-S(O)₂Me—C₆H₄) | 595 |
| | pyridin-3-yl) | | | | | |
| 22 | (CH₂)₂N(C₆H₅)CO(2-OH-5-F— | 1 | CO | Et | CH₂(4-S(O)₂Me—C₆H₄) | 580 (M-H) |
| | C₆H₃) | | | | | |
| 23 | (CH₂)₂CH(C₆H₅)NH₂ | 1 | CO | Et | CH₂(4-S(O)₂Me—C₆H₄) | 458 |
| 24 | (CH₂)₂NHC₆H₅ | 1 | CO | Et | CH₂(4-S(O)₂Me—C₆H₄) | 444 |
| 25 | (CH₂)₂NHC₆H₅ | 1 | CO | Et | CH₂(4-F—C₆H₄) | 384 |
| 26 | (CH₂)₂CH(OH)C₆H₅ | 1 | CO | Et | CH₂(4-S(O)₂Me—C₆H₄) | 459 |
| 27 | CH(Me)CH₂CH(C₆H₅)₂ | 1 | CO | Et | CH₂(4-S(O)₂Me—C₆H₄) | 533 |
| 28 | CH(Me)(CH₂)₂C₆H₅ | 1 | CO | Et | CH₂(4-S(O)₂Me—C₆H₄) | 457 |
| 29 | (CH₂)₂CH(Me)(3-CF₃—C₆H₄) | 1 | CO | Et | CH₂(4-S(O)₂Me—C₆H₄) | 525 |
| 30 | (CH₂)₂CH(Me)(3-Cl—C₆H₄) | 1 | CO | Et | CH₂(4-S(O)₂Me—C₆H₄) | 491 |
| 31 | (CH₂)₂CH(Me)C₆H₅ | 1 | CO | Et | CH₂(4-S(O)₂Me—C₆H₄) | 457 |
| 32 | (CH₂)₂CH(Me)(3,4-Cl₂—C₆H₃) | 1 | CO | Et | CH₂(4-S(O)₂Me—C₆H₄) | 525 |
| 33 | (CH₂)₂CH(C₆H₅)₂ | 0 | CO | Et | CH₂(4-S(O)₂Me—C₆H₄) | 477 |
| 34 | (CH₂)₂CH(4-Cl—C₆H₄)4-pyridyl | 1 | CO | Et | CH₂(4-S(O)₂Me—C₆H₄) | 554 |
| 35 | (CH₂)₂CH(4-Cl—C₆H₄)2-pyridyl | 1 | CO | Et | CH₂(4-S(O)₂Me—C₆H₄) | 554 |
| 36 | (CH₂)₂CH(C₆H₅)-(1,3- | 1 | CO | Et | CH₂(4-S(O)₂Me—C₆H₄) | 563 |
| | benzodioxol-5-yl) | | | | | |
| 37 | (CH₂)₂CH(C₆H₅)(4-Cl—C₆H₄) | 1 | CO | Et | CH₂(4-S(O)₂Me—C₆H₄) | 553 |
| 38 | (CH₂)₂CH(C6115)(3,4-Cl₂—C₆H₃) | 1 | CO | Et | CH₂(4-S(O)₂Me—C₆H₄) | 587 |
| 39 | (CH₂)₂CH(C₆H₅)(4-MeO—C₆H₄) | 1 | CO | Et | CH₂(4-S(O)₂Me—C₆H₄) | 549 |
| 40 | (CH₂)₂CH(C₆H₅)(3-Cl—C₆H₄) | 1 | CO | Et | CH₂(4-S(O)₂Me—C₆H₄) | 553 |
| 41 | (CH₂)₂CH(C₆H₅)(4-Me—C₆H₄) | 1 | CO | Et | CH₂(4-S(O)₂Me—C₆H₄) | 533 |
| 42 | (CH₂)₂CH(C₆H₅)(4-CF₃—C₆H₄) | 1 | CO | Et | CH₂(4-S(O)₂Me—C₆H₄) | 587 |
| 43 | (CH₂)₂CH(4-F—C₆H₄)₂ | 1 | CO | Et | CH₂(4-S(O)₂Me—C₆H₄) | 555 |
| 44 | (CH₂)₂CH(4-F—C₆H₄)₂ | 1 | CO | CH₂CH═CH₂ | CH₂(4-S(O)₂Me—C₆H₄) | 567 |

‡Ref: Stefan Sanczuk, Hubert K. F. Hermans (Janssen Pharmaceutica N. V., Beig.). Chemical Abstracts 87: 53094.

TABLE IV
Table IV discloses compounds of formula (Id):

| | |
|---|---|
| | (Id) |
| 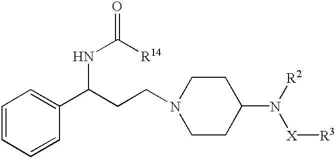 | |

wherein the variables R¹⁴, X, R² and R³ are as defined in the Table below.
Mass Spectrum details are given for certain compounds in Table IV.

| Compound | | | | | LCMS |
|---|---|---|---|---|---|
| No. | X | R² | R³ | R¹⁴ | (MH+) |
| 1 | CO | Et | CH₂(4-S(O)₂Me—C₆H₄) | phenyl | 562 |
| 2 | CO | Et | CH₂(4-S(O)₂Me—C₆H₄) | iso-Pr | 528 |
| 3 | CO | Et | CH₂(4-S(O)₂Me—C₆H₄) | CH(CH₂CH₃)₂ | 556 |
| 4 | CO | Et | CH₂(4-S(O)₂Me—C₆H₄) | CH(CH₃)CH₂CH₂CH₃ | 556 |
| 5 | CO | Et | CH₂(4-S(O)₂Me—C₆H₄) | CH₂C(CH₃)₃ | 556 |
| 6 | CO | Et | CH₂(4-S(O)₂Me—C₆H₄) | CH₂CH(CH₃)₂ | 542 |
| 7 | CO | Et | CH₂(4-S(O)₂Me—C₆H₄) | CH₂CH(CH₃)CH₂CH₃ | 556 |
| 8 | CO | Et | CH₂(4-S(O)₂Me—C₆H₄) | Et | 514 |
| 9 | CO | Et | CH₂(4-S(O)₂Me—C₆H₄) | CH₂CH₂CH(CH₃)₂ | 556 |
| 10 | CO | Et | CH₂(4-S(O)₂Me—C₆H₄) | n-Pr | 528 |
| 11 | CO | Et | CH₂(4-S(O)₂Me—C₆H₄) | 1-Me-pyrrol-2-yl | 565 |
| 12 | CO | Et | CH₂(4-S(O)₂Me—C₆H₄) | furan-2-yl | 552 |
| 13 | CO | Et | CH₂(4-S(O)₂Me—C₆H₄) | tert-Bu | 542 |
| 14 | CO | Et | CH₂(4-S(O)₂Me—C₆H₄) | C(CH₃)₂CH₂CH₃ | 556 |
| 15 | CO | Et | CH₂(4-S(O)₂Me—C₆H₄) | CH₂OEt | 544 |
| 16 | CO | Et | CH₂(4-S(O)₂Me—C₆H₄) | n-Bu | 542 |
| 17 | CO | Et | CH₂(4-S(O)₂Me—C₆H₄) | n-pentyl | 556 |
| 18 | CO | Et | CH₂(4-S(O)₂Me—C₆H₄) | C(OH)Me₂ | 544 |
| 19 | CO | Et | CH₂(4-S(O)₂Me—C₆H₄) | pyrrol-2-yl | 551 |
| 20 | CO | Et | CH₂(4-S(O)₂Me—C₆H₄) | furan-3-yl | 552 |
| 21 | CO | Et | CH₂(4-S(O)₂Me—C₆H₄) | thien-2-yl | 568 |
| 22 | CO | Et | CH₂(4-S(O)₂Me—C₆H₄) | thien-3-yl | 568 |
| 23 | CO | Et | CH₂(4-S(O)₂Me—C₆H₄) | pyrazin-2-yl | 564 |
| 24 | CO | Et | CH₂(4-S(O)₂Me—C₆H₄) | pyridin-2-yl | 563 |
| 25 | CO | Et | CH₂(4-S(O)₂Me—C₆H₄) | pyridin-3-yl | 563 |
| 26 | CO | Et | CH₂(4-S(O)₂Me—C₆H₄) | pyridin-4-yl | 563 |
| 27 | CO | Et | CH₂(4-S(O)₂Me—C₆H₄) | 3-Me-furan-2-yl | 566 |
| 28 | CO | Et | CH₂(4-S(O)₂Me—C₆H₄) | CH₂CH₂OMe | 544 |
| 29 | CO | Et | CH₂(4-S(O)₂Me—C₆H₄) | CH₂CH₂OEt | 558 |
| 30 | CO | Et | CH₂(4-S(O)₂Me—C₆H₄) | CH(OH)CH₂CH₂CH₃ | 558 |
| 31 | CO | Et | CH₂(4-S(O)₂Me—C₆H₄) | 2-Me-furan-3-yl | 566 |
| 32 | CO | Et | CH₂(4-S(O)₂Me—C₆H₄) | 4-Me-oxazol-5-yl | 567 |
| 33 | CO | Et | NHCH₂C₆H₅ | azetidin-1-yl | |
| 34 | CO | Et | NHCH₂(4-F—C₆H₄) | azetidin-1-yl | |
| 35 | CO | Et | NNCH₂(4-S(O)₂Me—C₆H₄) | azetidin-1-yl | |
| 36 | CO | Et | NHCH₂(4-S(O)₂NH₂—C₆H₄) | azetidin-1-yl | |
| 37 | CO | Et | CH₂C₆H₅ | azetidin-1-yl | |
| 38 | CO | Et | CH₂(4-F—C₆H₄) | azetidin-1-yl | |
| 39 | CO | Et | CH₂(4-S(O)₂Me—C₆H₄) | azetidin-1-yl | |
| 40 | CO | Et | CH₂(4-S(O)₂NH₂C₆H₄) | azetidin-1-yl | |
| 41 | CO | allyl | NHCH₂C₆H₅ | azetidin-1-yl | |
| 42 | CO | allyl | NHCH₂(4-F—C₆H₄) | azetidin-1-yl | |
| 43 | CO | allyl | NHCH₂(4-S(O)₂Me—C₆H₄) | azetidin-1-yl | |
| 44 | CO | allyl | NHCH₂(4-S(O)₂NH₂—C₆H₄) | azetidin-1-yl | |
| 45 | CO | allyl | CH₂C₆H₅ | azetidin-1-yl | |
| 46 | CO | allyl | CH₂(4-F—C₆H₄) | azetidin-1-yl | |
| 47 | CO | allyl | CH₂(4-S(O)₂Me—C₆H₄) | azetidin-1-yl | |
| 48 | CO | allyl | CH₂(4-S(O)₂NH₂—C₆H₄) | azetidin-1 -yl | |
| 49 | CO | cPr | NHCH₂C₆H₅ | azetidin-1-yl | |
| 50 | CO | cPr | NHCH₂(4-F—C₆H₄) | azetidin-1-yl | |
| 51 | CO | cPr | NHCH₂(4-S(O)₂Me—C₆H₄) | azetidin-I-yl | |
| 52 | CO | cPr | NHCH₂(4-S(O)₂NH₂—C₆H₄) | azetidin-1-yl | |
| 53 | CO | cPr | CH₂C₆H₅ | azetidin-1-yl | |
| 54 | CO | cPr | CH₂(4-F—C₆H₄) | azetidin-1-yl | |
| 55 | CO | cPr | CH₂(4-S(O)₂Me—C₆H₄) | azetidin-1-yl | |
| 56 | CO | cPr | CH₂(4-S(O)₂NH₂—C₆H₄) | azetidin-1-yl | |
| 57 | CO | Et | CH₂(4-S(O)₂Me—C₆H₄) | 2-F—C₆H₄ | 580 |
| 58 | CO | Et | CH₂(4-S(O)₂Me—C₆H₄) | 2,6-F₂—C₆H₃ | 598 |
| 59 | CO | Et | CH₂(4-S(O)₂Me—C₆H₄) | 2-Cl—C₆H₄ | 596 |
| 60 | CO | Et | CH₂(4-S(O)₂Me—C₆H₄) | 2-MeO—C₆H₄ | 592 |
| 61 | CO | Et | CH₂(4-S(O)₂Me—C₆H₄) | 3-CN—C₆H₄ | 587 |
| 62 | CO | Et | CH₂(4-S(O)₂Me—C₆H₄) | 3-F—C₆H₄ | 580 |
| 63 | CO | Et | CH₂(4-S(O)₂Me—C₆H₄) | 3-MeO—C₆H₄ | 592 |
| 64 | CO | Et | CH₂(4-S(O)₂Me—C₆H₄) | 3-Me—C₆H₄ | 576 |
| 65 | CO | Et | CH₂(4-S(O)₂Me—C₆H₄) | 4-CN—C₆H₄ | 587 |
| 66 | CO | Et | CH₂(4-S(O)₂Me—C₆H₄) | 4-F—C₆H₄ | 580 |
| 67 | CO | Et | CH₂(4-S(O)₂Me—C₆H₄) | 4-Cl—C₆H₄ | 596 |
| 68 | CO | Et | CH₂(4-S(O)₂Me—C₆H₄) | 4-(COCH₃)C₆H₄ | 604 |
| 69 | CO | Et | CH₂(4-S(O)₂Me—C₆H₄) | 4-Me—C₆H₄ | 576 |
| 70 | CO | Et | CH₂(4-S(O)₂Me—C₆H₄) | CH(Me)C₆H₅ | 590 |
| 71 | CO | Et | CH₂(4-S(O)₂Me—C₆H₄) | CH₂(2-F—C₆H₄) | 594 |
| 72 | CO | Et | CH₂(4-S(O)₂Me—C₆H₄) | CH₂(2-MeO—C₆H₄) | 606 |
| 73 | CO | Et | CH₂(4-S(O)₂Me—C₆H₄) | CH₂(3-MeO—C₆H₄) | 606 |
| 74 | CO | Et | CH₂(4-S(O)₂Me—C₆H₄) | CH₂(4-F—C₆H₄) | 594 |
| 75 | CO | Et | CH₂(4-S(O)₂Me—C₆H₄) | CH₂(4-MeO—C₆H₄) | 606 |
| 76 | CO | Et | CH₂(4-S(O)₂Me—C₆H₄) | indol-5-yl | 601 |
| 77 | CO | Et | CH₂(4-S(O)₂Me—C₆H₄) | 6-Cl-pyridin-3-yl | 597 |
| 78 | CO | Et | CH₂(4-S(O)₂Me—C₆H₄) | 2-NO₂—C₆H₄ | 607 |
| 79 | CO | Et | CH₂(4-S(O)₂Me—C₆H₄) | 3-NO₂—C₆H₄ | 607 |
| 80 | CO | Et | CH₂(4-S(O)₂Me—C₆H₄) | 4-NO₂—C₆H₄ | 607 |
| 81 | CO | Et | CH₂(4-S(O)₂Me—C₆H₄) | 3,4-F₂—C₆H₃ | 598 |
| 82 | CO | Et | CH₂(4-S(O)₂Me—C₆H₄) | benztriazol-4-yl | 603 |
| 83 | CO | Et | CH₂(4-S(O)₂Me—C₆H₄) | 2-Me-pyridin-3-yl | 577 |
| 84 | CO | Et | CH₂(4-S(O)₂Me—C₆H₄) | 6-Me-pyridin-2-yl | 577 |
| 85 | CO | Et | CH₂(4-S(O)₂Me—C₆H₄) | CH(OMe)C₆H₅ | 606 |
| 86 | CO | Et | CH₂(4-S(O)₂Me—C₆H₄) | 5-Me-pyrazin-2-yl | 578 |
| 87 | CO | Et | CH₂(4-S(O)₂Me—C₆H₄) | dihydrobenzofuran-4-yl | 604 |
| 88 | CO | Et | CH₂(4-S(O)₂Me—C₆H₄) | 2-OMe-pyridin-3-yl | 593 |
| 89 | CO | Et | CH₂(4-S(O)₂Me—C₆H₄) | 6-Cl-pyridin-2-yl | 597 |
| 90 | CO | Et | CH₂(4-S(O)₂Me—C₆H₄) | 2-Cl-pyridin-4-yl | 597 |
| 91 | CO | Et | CH₂(4-S(O)₂Me—C₆H₄) | 1H-pyridin-2-on-6-yl | 579 |
| 92 | CO | Et | CH₂(4-S(O)₂Me—C₆H₄) | indol-7-yl | 601 |
| 93 | CO | Et | CH₂(4-S(O)₂Me—C₆H₄) | dihydrobenzofuran-7-yl | 604 |
| 94 | CO | Et | CH₂(4-S(O)₂Me—C₆H₄) | 6-CN-pyridin-3-yl | 588 |
| 95 | CO | Et | CH₂(4-S(O)₂Me—C₆H₄) | 2-F-pyridin-3-yl | 581 |

The following abbreviations are used in Tables I to IV:

| | | | |
|---|---|---|---|
| Me = | methyl | Et = | ethyl |
| Pr = | propyl | Bu = | butyl |
| cPr = | cyclopropyl | cBu = | cyclobutyl |

The compounds of formula (I), (Ia), (Ib), (Ic) or (Id) can be prepared as shown in the processes on pages marked Schemes 1 to 14 below. (In Scheme 10 suitable coupling agents include HATU (O-(7-Azabenzotriazol-1-yl)-N,N,N′,N′-tetramethyluronium hexafluorophosphate) and PyBROP (bromo-tris-pyrrolidinophosphonium hexafluorophosphate) which may be employed according to Example 26.) The starting materials for these processes are either commercially available or can be prepared either by literature methods or by adapting literature methods. In the Schemes the variables R^{1*}, R^{2*} and R^{3*} have been used where the group R¹, R² or R³ is, respectively, CH₂R^{1*}, CH₂R^{2*} or CH₂R^{3*}; Ac is CH₃C(O); and Ar¹ and Ar² denote aromatic rings which are optionally substituted. Although Schemes 1-14 are depicted for m and p=1, and R⁴, R⁵, R⁶ and R⁷ as hydrogen, it is clear that they can be readily adapted for alternative values of m, p, R⁴, R⁵, R⁶ and R⁷.

In a further aspect the invention provides processes for preparing the compounds of formula (I), (Ia), (Ib), (Ic) and (Id). Many of the intermediates in the processes are novel and these are provided as further features of the invention.

The compounds of the invention have activity as pharmaceuticals, in particular as modulators (such as agonists, partial agonists, inverse agonists or antagonists) of chemokine receptor (especially CCR5) activity, and may be used in the treatment of autoimmune, inflammatory, proliferative or hyperproliferative diseases, or immunologically-mediated diseases (including rejection of transplanted organs or tissues and Acquired Immunodeficiency Syndrome (AIDS)). Examples of these conditions are:

(1) (the respiratory tract) obstructive diseases of airways including: chronic obstructive pulmonary disease (COPD) (such as irreversible COPD); pulmonary fibrosis; asthma {such as bronchial, allergic, intrinsic, extrinsic or dust asthma, particularly chronic or inveterate asthmia (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis; sarcoidosis; farmer's lung and related diseases; nasal polyposis; fibroid lung or idiopathic interstitial pneumonia;

(2) (bone and joints) artbrides including rheumatic, infectious, autoimmune, seronegative spondyloarthropathies (such as ankylosing spondylitis, psoriatic arthritis or Reiter's disease), Behcet's disease, Sjogren's syndrome or systemic sclerosis;

(3) (skin and eyes) psoriasis, atopic dermatitis, contact dermatitis or other eczmatous dermitides, seborrhoetic dermatitis, Lichen planus, Phemphigus, bullous Phemphigus, Epidermolysis bullosa, urticaria, angiodermas, vasculitides erythemas, cutaneous eosinophilias, uveitis, Alopecia areata or vernal conjunctivitis;

(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);

(5) (Allograft rejection) acute and chronic following, for example, transplantation of kidney, heart, liver, lung, bone marrow, skin or cornea; or chronic graft versus host disease; and/or

(6) (other tissues or diseases) Alzheimer's disease, multiple sclerosis, atherosclerosis, inhibiting the entry of viruses into target cells, Acquired Immunodeficiency Syndrome (AIDS), Lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, Sezary syndrome, idiopathic thrombocytopenia pupura, disorders of the menstrual cycle, glomerulonephritis or cerebral malaria.

The compounds of the present invention are also of value in inhibiting the entry of viruses (such as human immunodeficiency virus (HIV)) into target calls and, therefore, are of value in the prevention of infection by viruses (such as HIV), the treatment of infection by viruses (such as HIV) and the prevention and/or treatment of acquired immune deficiency syndrome (AIDS).

According to a further feature of the invention there is provided a compound of the formula (I), (Ia), (Ib), (Ic) or (Id), or a pharmaceutically acceptable salt thereof or a solvate thereof, for use in a method of treatment of a warm blooded animal (such as man) by therapy (including prophylaxis).

According to a further feature of the present invention there is provided a method for modulating chemokine receptor activity (especially CCR5 receptor activity) in a warm blooded animal, such as man, in need of such treatment, which comprises administering to said animal an effective amount of a compound of the present invention, or a pharmaceutically acceptable salt thereof or a solvate thereof.

The present invention also provides the use of a compound of the formula (I), (Ia), (Ib), (Ic) or (Id), or a pharmaceutically acceptable salt thereof or a solvate thereof, as a medicament, especially a medicament for the treatment of transplant rejection, respiratory disease, psoriasis or rheumatoid arthritis (especially rheumatoid arthritis). [Respiratory disease is, for example, COPD, asthma {such as bronchial, allergic, intrinsic, extrinsic or dust asthma, particularly chronic or inveterate asthma (for example late asthma or airways hyper-responsiveness)} or rhinitis {acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis}; and is particularly asthma or rhinitis].

In another aspect the present invention provides the use of a compound of the formula (I), (Ia), (Ib), (Ic) or (Id), or a pharmaceutically acceptable salt thereof or a solvate thereof, in the manufacture of a medicament for use in therapy (for example modulating chemokine receptor activity (especially CCR5 receptor activity (especially rheumatoid arthritis)) in a warm blooded animal, such as man).

The invention also provides a compound of the formula (I), (Ia), (Ib), (Ic) or (Id), or a pharmaceutically acceptable salt thereof or a solvate thereof, for use as a medicament, especially a medicament for the treatment of rheumatoid arthritis.

In another aspect the present invention provides the use of a compound of the formula (I), (Ia), (Ib) or (Ic), or a pharmaceutically acceptable salt thereof or a solvate thereof, in the manufacture of a medicament for use in therapy (for example modulating chemokine receptor activity (especially CCR5 receptor activity (especially rheumatoid arthritis)) in a warm blooded animal, such as man).

The invention further provides the use of a compound of formula (I), (Ia), (Ib), (Ic) or (Id), or a pharmaceutically acceptable salt thereof, in the manufacture of a medicament for use in the treatment of:

(1) (the respiratory tract) obstructive diseases of airways including: chronic obstructive pulmonary disease (COPD) (such as irreversible COPD); asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis; sarcoidosis; farmer's lung and related diseases; nasal polyposis; fibroid lung or idiopathic interstitial pneumonia;

(2) (bone and joints) arthrides including rheumatic, infectious, autoimmune, seronegative spondyloarthropathies (such as ankylosing spondylitis, psoriatic arthritis or Reiter's disease), Behget's disease, Sjogren's syndrome or systemic sclerosis;

(3) (skin and eyes) psoriasis, atopic dermatitis, contact dermatitis or other eczmatous dermitides, seborrhoetic dermatitis, Lichen planus, Phemphigus, bullous Phemphigus, Epidermolysis bullosa, urticaria, angiodermas, vasculitides erythemas, cutaneous eosinophilias, uveitis, Alopecia areata or vernal conjunctivitis;

(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);

(5) (Allograft rejection) acute and chronic following, for example, transplantation of kidney, heart, liver, lung, bone marrow, skin or cornea; or chronic graft versus host disease; and/or

(6) (other tissues or diseases) Alzheimer's disease, multiple sclerosis, atherosclerosis, Acquired Immunodeficiency Syndrome (AIDS), Lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, Sezary syndrome, idiopathic thrombocytopenia pupura or disorders of the menstrual cycle;

in a warm blooded animal, such as man.

The present invention further provides a method of treating a chemokine mediated disease state (especially a CCR5 mediated disease state) in a warm blooded animal, such as man, which comprises administering to a mammal in need of such treatment an effective amount of a compound of formula (I), (Ia), (Ib), (Ic) or (Id), or a pharmaceutically acceptable salt thereof or solvate thereof.

In order to use a compound of the invention, or a pharmaceutically acceptable salt thereof or solvate thereof, for the therapeutic treatment of a warm blooded animal, such as man, in particular modulating chemokine receptor (for example CCR5 receptor) activity, said ingredient is normally formulated in accordance with standard pharmaceutical practice as a pharmaceutical composition.

Therefore in another aspect the present invention provides a pharmaceutical composition which comprises a compound of the formula (I), (Ia), (Ib), (Ic) or (Id), or a pharmaceutically acceptable salt thereof or a solvate thereof (active ingredient), and a pharmaceutically acceptable adjuvant, diluent or carrier. In a further aspect the present invention provides a process for the preparation of said composition which comprises mixing active ingredient with a pharmaceutically acceptable adjuvant, diluent or carrier. Depending on the mode of administration, the pharmaceutical composition will preferably comprise from 0.05 to 99% w (per cent by weight), more preferably from 0.05 to 80% w, still more preferably from 0.10 to 70% w, and even more preferably from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), oral, rectal or parenteral administration. For these purposes the compounds of this invention may be formulated by means known in the art into the form of, for example, aerosols, dry powder formulations, tablets, capsules, syrups, powders, granules, aqueous or oily solutions or suspensions, (lipid) emulsions, dispersible powders, suppositories, ointments, creams, drops and sterile injectable aqueous or oily solutions or suspensions.

A suitable pharmaceutical composition of this invention is one suitable for oral administration in unit dosage form, for example a tablet or capsule which contains between 0.1 mg and 1 g of active ingredient.

In another aspect a pharmaceutical composition of the invention is one suitable for intravenous, subcutaneous or intramuscular injection.

Each patient may receive, for example, an intravenous, subcutaneous or intramuscular dose of 0.01 mgkg⁻¹ to 100 mgkg⁻⁷ of the compound, preferably in the range of 0.1 mgkg⁻¹ to 20 mgkg⁻¹ of this invention, the composition being administered 1 to 4 times per day. The intravenous, subcutaneous and intramuscular dose may be given by means of a bolus injection. Alternatively the intravenous dose may be given by continuous infusion over a period of time. Alternatively each patient will receive a daily oral dose which is approximately equivalent to the daily parenteral dose, the composition being administered 1 to 4 times per day.

The following illustrate representative pharmaceutical dosage forms containing the compound of formula (I), (Ia), (Ib), (Ic) or (Id), or a pharmaceutically acceptable salt thereof or a solvent thereof (hereafter Compound X), for therapeutic or prophylactic use in humans:

(a)

| Tablet I | mg/tablet |
|---|---|
| Compound X | 100 |
| Lactose Ph. Eur. | 179 |
| Croscarmellose sodium | 12.0 |
| Polyvinylpyrrolidone | 6 |
| Magnesium stearate | 3.0 |

(b)

| Tablet II | mg/tablet |
|---|---|
| Compound X | 50 |
| Lactose Ph. Eur. | 229 |
| Croscarmellose sodium | 12.0 |
| Polyvinylpyrrolidone | 6 |
| Magnesium stearate | 3.0 |

(c)

| Tablet III | mg/tablet |
|---|---|
| Compound X | 1.0 |
| Lactose Ph. Eur. | 92 |
| Croscarmellose sodium | 4.0 |
| Polyvinylpyrrolidone | 2.0 |
| Magnesium stearate | 1.0 |

(d)

| Capsule | mg/capsule |
|---|---|
| Compound X | 10 |
| Lactose Ph. Eur. | 389 |
| Croscarmellose sodium | 100 |
| Magnesium stearate | 1.0 |

(e)

| Injection I | (50 mg/ml) |
|---|---|
| Compound X | 5.0% w/v |
| Isotonic aqueous solution | to 100% |

Buffers, pharmaceutically-acceptable cosolvents such as polyethylene glycol, polypropylene glycol, glycerol or ethanol or complexing agents such as hydroxy-propyl β-cyclodextrin may be used to aid formulation.

The above formulations may be obtained by conventional procedures well known in the pharmaceutical art. The tablets (a)-(c) may be enteric coated by conventional means, for example to provide a coating of cellulose acetate phthalate.

The invention will now be illustrated by the following non-limiting examples in which, unless stated otherwise:

(i) temperatures are given in degrees Celsius (° C.); operations were carried out at room or ambient temperature, that is, at a temperature in the range of 18-25° C.;

(ii) organic solutions were dried over anhydrous magnesium sulphate; evaporation of solvent was carried out using a rotary evaporator under reduced pressure (600-4000 Pascals; 4.5-30 mm Hg) with a bath temperature of up to 60° C.;

(iii) chromatography unless otherwise stated means flash chromatography on silica gel; thin layer chromatography (TLC) was carried out on silica gel plates; where a “Bond Elut” column is referred to, this means a column containing 10 g or 20 g of silica of 40 micron particle size, the silica being contained in a 60 ml disposable syringe and supported by a porous disc, obtained from Varian, Harbor City, Calif., USA under the name “Mega Bond Elut SI”. Where an “Isolute™ SCX column” is referred to, this means a column containing benzenesulphonic acid (non-endcapped) obtained from International Sorbent Technology Ltd., 1st House, Duffryn Industial Estate, Ystrad Mynach, Hengoed, Mid Clamorgan, UK. Where “Argonaut™ PS-tris-amine scavenger resin” is referred to, this means a tris-(2-aminoethyl)amine polystyrene resin obtained from Argonaut Technologies Inc., 887 Industrial Road, Suite G, San Carlos, Calif., USA.

(iv) in general, the course of reactions was followed by TLC and reaction times are given for illustration only;

(v) yields, when given, are for illustration only and are not necessarily those which can be obtained by diligent process development; preparations were repeated if more material was required;

(vi) when given, ¹H NMR data is quoted and is in the form of delta values for major diagnostic protons, given in parts per million (ppm) relative to tetramethylsilane (TMS) as an internal standard, determined at 300 MHz using perdeuterio DMSO (CD₃SOCD₃) as the solvent unless otherwise stated; coupling constants (J) are given in Hz;

(vii) chemical symbols have their usual meanings; SI units and symbols are used;

(viii) solvent ratios are given in percentage by volume;

(ix) mass spectra (MS) were run with an electron energy of 70 electron volts in the chemical ionisation (APCI) mode using a direct exposure probe; where indicated ionisation was effected by electrospray (ES); where values for m/z are given, generally only ions which indicate the parent mass are reported, and unless otherwise stated the mass ion quoted is the positive mass ion—(M+H)⁺;

(x) LCMS characterisation was performed using a pair of Gilson 306 pumps with Gilson 233 XL sampler and Waters ZMD4000 mass spectrometer. The LC comprised water symmetry 4.6×50 column C18 with 5 micron particle size. The eluents were: A, water with 0.05% formic acid and B, acetonitrile with 0.05% formic acid. The eluent gradient went from 95% A to 95% B in 6 minutes. Where indicated ionisation was effected by electrospray (ES); where values for m/z are given, generally only ions which indicate the parent mass are reported, and unless otherwise stated the mass ion quoted is the positive mass ion—(M+H)⁺ and

(xi) the following abbreviations are used:

| | |
|---|---|
| DMSO | dimethyl sulphoxide; |
| DMF | N-dimethylformamide; |
| DCM | dichloromethane; |
| THF | tetrahdydrofuran; |
| DIPEA | N,N-diisopropylethylamine; |
| NMP | N-methylpyrrolidinone; |
| HATU | O-(7-Azabenzotriazol-1-yl)-N,N,N′,N′-tetramethyluronium |
| | hexafluorophosphate; |
| Boc | tert-butoxycarbonyl |
| MeOH | methanol; |
| EtOH | ethanol; and |
| EtOAc | ethyl acetate. |

### EXAMPLE 1

This Example illustrates the preparation of N-[1-(3,3-diphenylpropyl)-4-piperidinyl]-N-methylisonicotinamide (Compound No. 1 of Table I).

To a solution of isonicotinic acid (0.6 mg, 5 μM) in NMP (50 μL) was added a solution of 4-methylamino-1-(3,3-diphenylpropyl)piperidine dihydrochloride (Method A) (1.9 mg, 5 μM) and diisopropylethylamine (8 μL, 45 μM) in NMP (50 μL) followed by a solution of bromo-tris-pyrrolidinophosphonium hexafluorophosphate (4.7 mg, 10 μM) in NMP (100 μL). After 15 h the reaction mixture was concentrated to give the title compound which was characterised by LCMS; MS: 415.

The method of Example 1 can be repeated using different acids in place of isonicotinic acid, or different piperidines (such as 4-methylamino-1-(3-R/S-phenylbutyl)piperidine dihydrochloride (Method B), 4-propargylamino-1-(3-R/S-phenylbutyl)piperidine (Method C), 4-allylamino-1-(3,3-diphenylpropyl)piperidine (Method D), 4-allylamino-1-(3-R/S-phenylbutyl)piperidine (Method E) or 4-(cyclopropylmethyl)amino-1-(3-R/S-phenylbutyl)piperidine (Method R)) in place of 4-methylamino-1-(3,3-diphenylpropyl)piperidine dihydrochloride.

### EXAMPLE 2

This Example illustrates the preparation of N′-(2,4-difluorophenyl)-N-[1-(2,6-dimethoxybenzyl)piperidin-4-yl]-N-phemethylurea (Compound No. 1 of Table III).

To a solution of 2,6-dimethoxybenzaldehyde (1.7 mg, 10 μM) in NMP (100 μL) was added a solution of 4-piperidinyl-N-(2-phenylethyl)-2,4-difluorophenylurea.trifluoroacetic acid (Method F) (2.4 mg, 5 μM) and diisopropylethylamine (1 μL, 5.5 μM) in NMP (100 μL). After 1.5 h a solution of sodium triacetoxyborohydride (2.8 mg, 15 μM) in acetonitrile: NMP, 1:1 (100 μL) was added. After 16 h at room temperature the reaction mixture was concentrated to give the title compound which was characterised by LCMS; MS: 510.

The procedure described in Example 2 can be repeated using different aldehydes in place of 2,6-dimethoxybenzaldehyde or other piperidines (such as 4-methylamino-1-(3,3-diphenylpropyl)piperidine.dihydrochloric acid (Method A) or 4-amino-1-(3,3-diphenylpropyl)piperidine.ditrifluoroacetic acid (Method G)) in place of 4-piperidinyl-N-(2-phenylethyl)-2,4-difluorophenylurea trifluoroacetic acid.

### EXAMPLE 3

This Example illustrates the preparation of N-[1-(3,3-diphenylpropyl)-piperidin4-yl]-N-methyl-2-(trifluoromethoxy)benzenesulphonamide (Compound No. 53 of Table I).

To a solution of 2-trifluoromethoxybenzenesulphonyl chloride (1.3 mg, 5 μM) in acetonitrile (50 μL) was added a solution of 4-methylamino-1-(3,3-diphenylpropyl)-piperidine.dihydrochloride (Method A) (1.9 mg, 5 μM) and N,N-diisopropylethylamine (1.8 μL, 10 μM) in pyridine (50 μL). After 15 h the reaction mixture was concentrated to give the title compound which was characterised by LCMS; MS: 533.

The procedure described in Example 3 can be repeated using different sulphonylchlorides (such as 4-acetamido,3-chlorobenzenesulphonyl chloride) in place of 2-trifluoromethoxybenzenesulphonyl chloride or different piperidines (such as 4amino-1-(3,3-diphenylpropyl)piperidine.ditrifluoroacetic acid (Method G)) in place of 4-methylamino-1-(3,3-diphenylpropyl)piperidine dihydrochloride.

### EXAMPLE 4

This Example illustrates the preparation of N′-(3,4-dichlorophenyl)-N-[1-(3,3-diphenylpropyl)piperidin-4-yl]-N-methylurea (Compound No. 68 of Table I).

A solution of 4-methylamino-1-(3,3-diphenylpropyl)piperidine.dihydrochloride (Method A) (1.9 mg, 5 μM) and DIPEA (1.8 μL, 10 μM) in DCM (100 μL) was added to 3,4-dichlorophenylisocyanate (19 mg, 0.1 mM). After 15 h DCM (800 μL) was added and Argonaut™ PS-tris-amine scavenger resin (0.66 g) was added and the reaction mixture agitated. The resin swelled considerably and the mixture was left to stand in order for the DCM to evaporate. Methanol (0.5 ml) was added and the mixture agitated; the organic layer was then transferred to another vessel and concentrated to give the title compound as an oil, which was characterised by LCMS; MS: 496.

The procedure described in Example 4 can be repeated using various isocyanates or carbamoyl chlorides in place of 3,4-dichlorophenylisocyanate or other piperidines (such as 4-amino-1-(3,3-diphenylpropyl)piperidine.ditrifluoroacetic acid (Method G), 4-amino-1-(3-R/S-phenylbutyl)piperidine ditrifluoroacetic acid salt (Method H)) in place of 4-methylamino-1-(3,3-diphenylpropyl)piperidine dihydrochloride.

### EXAMPLE 5

This Example illustrates the preparation of N-[1-(3,3-diphenylpropyl)-piperidin-4-yl]-N-methylthiophene-2-carboxamide (Compound No. 96 of Table I).

A solution of 4-methylamino-1-(3,3-diphenylpropyl)piperidine (the free base of the compound described in Method A) (0.1 g, 0.32 mmol) in dichloromethane (4.0 ml) was added to 2-thiophene carboxylic acid (1.0 mmol). To the resulting mixture was added a solution of diisopropylcarbodiimide (0.15 ml, 1.0 mmol) in dichloromethane (1.0 ml) followed by a solution of 1-hydroxybenzotriazole (0.135 g, 1.0 mmol) in DMF (2.0 ml) and the resulting mixture stirred at ambient temperature for 18 hours. The reaction mixture was then applied to an ISOLUTE™ SCX column (5 g) which was then washed with MeOH (30 ml) followed by a 1:4 mixture of aqueous ammonia and methanol (30 ml). Evaporation of the final wash gave the title compound as an oil (101 mg, 75% yield); MS: 419.

The procedure described in Example 5 can be repeated using different carboxylic acids in place of 2-thiophene carboxylic acid or other piperidines (such as 4-amino-1-(3,3-diphenylpropyl)piperidine (free base from Method G), 4-methylamino-1-(3-R/S-phenylbutyl)piperidine (free base from Method B) or 4-amino-1-(3-R/S-phenylbutyl)piperidine (free base from Method H)) in place of 4-methylamino-1-(3,3-diphenylpropyl)piperidine.

### EXAMPLE 6

This Example illustrates the preparation of N-[1-(3,3-diphenylpropyl)-4-piperidinyl]-(N-methyl)-3-chlorophenylurea (Compound 144 of Table I).

A solution of 4-methylamino-1-(3,3-diphenylpropyl)piperidine (the free base of the compound described in Method A) (0.1 g; 0.32 mmol) in DCM (4.0 ml) was added to 3-chlorophenyl isocyanate (1.0 mmol). The resulting mixture was stirred at ambient temperature for 18 hours. The reaction mixture was then applied to an ISOLUTE™ SCX column (5 g) which was then washed with methanol (30 ml) followed by a 1:4 mixture of aqueous ammonia and MeOH (30 ml). Evaporation of the final wash gave the product as an oil (112 mg, 76% yield); MS: 462.

The procedure described in Example 6 can be repeated using different isocyanates or carbamoyl chlorides in place of 3-chlorophenylisocyanate or other piperidines (such as 4-methylamino-1-(3-R/S-phenylbutyl)piperidine (free base from Method B)) in place of 4-methylamino-1-(3,3-diphenylpropyl)piperidine.

### EXAMPLE 7

This Example illustrates the preparation of N-[1-(3,3-diphenylpropyl)-4-piperidinyl]-N-methyl-4-(phenylmethoxy)phenylacetamide (Compound No. 268 of Table I).

To a solution of 4-methoxyphenylacetic acid (0.8 mg, 511 mol) in NMP (50 μL) was added a solution of 4-methylamino-1-(3,3-diphenylpropyl)piperidine dihydrochloride (Method A) (1.9 mg, 5 μmol) and DIPEA (8 μL, 45 μmol) in NMP (50 μL) followed by a solution of bromo-tris-pyrrolidino-phosphonium hexafluorophosphate (4.7 mg, 10 μmol) in NMP (100 μL). After 15 h the reaction mixture was concentrated to give the title compound which was characterised by LCMS; MS: 533.

### EXAMPLE 8

This Example illustrates the preparation of N-[1-(3,3-diphenylpropyl)-4-piperidinyl]-N-allyl-4-fluorophenylacetamide (Compound No. 269 of Table I).

To 4-fluorophenylacetic acid (1 mmol) was added 4-allylamino-1-(3,3-diphenylpropyl)piperidine (0.1 g; 0.3 mmol) in dichloromethane (2 ml). A solution of 1-hydroxybenztiazole (0.135 g; 0.1 mmol) in DMF (2 ml) and di-isopropyl-carbodiimide (0.126 ml; 1 mmol) in DCM was then added. The resulting mixture was stirred at room temperature overnight. The mixture was then applied to an ISOLUTE™ SCX cartridge (5 g) and washed with methanol (30 ml). The product was then eluted with 15% methylamine in ethanol. Purification was achieved by BondElut chromatography eluting with a solvent mixture of DCM to 5% methanol in DCM yielding the title compound (72 mg, 50%), which was characterised by LCMS; MS: 471.

### EXAMPLE 9

This Example illustrates the preparation of N-[1-(3,3-diphenylpropyl)-4-piperidinyl]-N-ethyl-4-trifluoromethoxyphenylacetamide (Compound No. 282 of Table I).

To a solution of 4-trifluoromethoxyphenylacetic acid (188 mg, 0.92 mmol) in dichloromethane (2 ml) was added 1-hydroxybenztriazole (124 mg) followed by diisopropylcarbodiimide (0.14 ml) and DMF (1 ml). The mixture was stirred at room temperature for 1 h, then a solution of 4-ethylamino-1-(3,3-diphenylpropyl)piperidine (147 mg, 0.46 mmol) in dichloromethane (2 ml) was added. The resulting mixture was stirred overnight then purified by eluting through an ISOLUTE™ SCX column with methanol followed by 2% aqueous ammonia in methanol. The product was then dissolved in ethyl acetate (2 ml) and treated with 1M HCl in diethyl ether (4 ml) giving the hydrochloride salt which was isolated by filtration, yielding N-[1-(3,3-diphenylpropyl)-4-piperidinyl]-N-ethyl-4-trifluoromethoxyphenylacetamide hydrochloride as a foam, 210 mg, 87%; NMR: 1.1 (m,3H), 1.7 (m, 2H), 2.1 (m, 2H), 3.0 (m, 4H), 3.5 (m, 5H), 3.8 (m, 4H), 4.3 (m, 1H), 7.1 (m, 2H), 7.3 (m, 12H); MS: 525.

### EXAMPLE 10

This Example illustrates the preparation of N′-(4-fluorophenylmethyl)-N-[1-(3,3-diphenylpropyl)-4-piperidinyl]-N-methylurea (Compound No. 388 of Table I).

To 4-fluorophenyl isocyanate (0.75 mmol) was added a solution of 4-methylamino-1-(3,3-diphenylpropyl)piperidine (0.19 g; 0.5 mmol) in DCM (4 ml). The resulting mixture was stirred at room temperature overnight. The resulting reaction mixture was then applied to an ISOLUTE™ SCX cartridge (5 g) and washed with methanol (30 ml). The product was then eluted using a 4:1 mixture of methanol and aqueous ammonia. Purification was achieved by BondElut chromatography eluting with a solvent mixture of DCM to 5% methanol in DCM to give the title compound (26 mg, 11%) which was characterised by LCMS; MS: 446.

### EXAMPLE 11

This Example illustrates the preparation of N′-(2,4-difluorophenyl)-N-[1-(3,3-diphenylpropyl)-4-piperidinyl]-N-phemethylurea (Compound No. 314 of Table I).

To a solution of N′-(2,4-difluorophenyl)-N-(4-piperidinyl)-N-phemethylurea trifluoroacetic acid salt (300 mg, 0.63 mmol) in DMF (5 ml) was added 3,3-diphenyl-1-bromopropane (360 mg, 1.26 mmol) followed by DIPEA (0.442 ml, 2.52 mmol). The resulting mixture was stirred at room temperature for 24 h. The reaction mixture was partitioned between water and dichloromethane, the organic phase was washed with water, dried (MgSO₄) and concentrated. The residue was purified by eluting through a silica gel cartridge with ethyl acetate followed by 5% ethanol in ethyl acetate to give the title compound as a gum, 80 mg; NMR: 1.6 (m, 6H), 4.9 (m, 5H), 2.2 (m, 3H), 2.8 (m, 3H), 3.9 (m, 2H), 7.0 (m, 1H), 7.2 (m, 15H), 7.4 (m, 1H), 8.0 (s, 1H); MS: 554.

### EXAMPLE 12

This Example illustrates the preparation of N′-(4-trifluoromethylphenylmethyl)-N-[1-(3,3-diphenylpropyl)-4-piperidinyl]-N-ethylurea (Compound No. 323 of Table I).

A solution of 4-trifluoromethylphenylacetic acid (0.8 mmol) in dry THF (2.0 ml) was cooled to 0° C. and triethylamine (0.11 ml; 0.8 mmol) in THF (1.0 ml) and diphenylphosphorylazide (0.17 ml; 0.8 mmol) in THF (2 ml) were added. Stirring was continued for 30 min. The mixture was allowed to warm to ambient temperature before toluene (5 ml) was added and the mixture heated to 100° C. for 1 h. After cooling to room temperature, a solution of4-ethylamino-1-(3,3-diphenylpropyl)piperidine (0.2 g; 0.6 mmol) in ethyl acetate (2 ml) was added and the mixture allowed to stir at room temperature for 72 h. The reaction mixture was then washed with aq. NaHCO₃ solution, dried and evaporated. Purification was by passage through a BondElut cartridge (Si) eluting with a gradient from 0-5% methanol in DCM, yielding the title compound (153 mg, 49%) which was characterised by LCMS; MS: 524.

### EXAMPLE 13

This Example illustrates the preparation of pyrrolidine carboxylic acid N-[1-(3,3-diphenylpropyl)-4-piperidinyl]-N-methyl amide (Compound No. 391 of Table I).

To diethylcarbamoyl chloride (0.75 mmol) was added a solution of 4-methylamino-1-(3,3-diphenylpropyl)piperidine (0.19 g; 0.5 mmol) in DCM (4 ml) followed by triethylamine (0.14 ml; 1 mmol). The resulting mixture was stirred at room temperature overnight. The resulting reation mixture was then applied to an ISOLUTE™ SCX cartridge (5 g) and washed with methanol (30 ml). The product was then eluted using a 4:1 mixture of methanol and 0.88 aqueous ammonia. Purification was achieved by BondElut chromatography eluting with a solvent mixture of DCM to 5% methanol in DCM to give the product (79 mg, 39%) which was characterised by LCMS; MS: 406.

### EXAMPLE 14

This Example illustrates the preparation of N-[1-(3,3-diphenylpropyl)-4-piperidinyl]-N-methyl-4-(cyclopropylaminosulfonyl)phenylacetamide (Compound No. 354 of Table I).

N-[1-(3,3-Diphenylpropyl)-4-piperidinyl]-N-methyl-4-fluorosulfonylphenylacetamide (0.005 mmol, in 100 μL MeCN) and cyclopropylamine (0.01 mmol in 100 μL MeCN) were mixed and allowed to stand overnight. The solvent was then evaporated to dryness under Genevac high vacuum.

### EXAMPLE 15

This Example illustrates the preparation of N-[1-(3,3-diphenylpropyl)-4-piperidinyl]-N-methyl-4-(2-hydroxyethylaminocarbonyl)phenylacetamide hydrochloride (Compound No. 385 of Table I).

A mixture of N-[1-(3,3-diphenylpropyl)-4-piperidinyl]-N-methyl-4-methoxycarbonylphenylacetaride (0.1 g; 0.2 mmol) was heated at 60° C. in a mixture of ethanolamine (1.0 mL) and acetonitrile (1.0 mL) for 12 hours. After cooling the mixture was partitioned between ethyl acetate (5 mL) and water (8 mL). The organic layer was washed a further twice with water and dried (Na₂SO₄) before purification on a silica BondElut, eluting with a gradient from 5-25% methanol in dichloromethane. The purified product was dissolved in ethyl acetate and treated with HCl in diethyl ether before evaporation to give the title compound as a solid (68 mg, 62%) which was characterised by LC-MS; MS: 514.

### EXAMPLE 16

This Example illustrates the preparation of 4(2-[4-methanesulfonylphenyl])-pentenoic acid N-[1-(3,3-diphenylpropyl)4-piperidinyl]amide hydrochloride salt (Compound No. 390 of Table I).

To a cooled (5° C.) solution of N-[1-(3,3-diphenylpropyl)-4-piperidinyl]-4-methanesulfonylphenylacetamide (1.61 g, 3.28 mmol) in DMF (1 mL) was added sodium hydride (131 mg 60% dispersion, 3.6 mmol). The resulting mixture was stirred for 5 minutes before the addition of allyl bromide (0.3 mL, 3.44 mmol). The reaction mixture was stirred at room temperature for 2 h then quenched with water. The mixture was extracted twice with ethyl acetate and the combined organic extracts were washed with water and brine, dried and evaporated. The residue was purified by silica gel chromatography (eluent 3% MeOH in DCM). The crude product was treated with ethereal HCl to afford the title compound (0.902 g); NMR (CDCl₃): 1.2 (m, 2H), 1.9 (m, 2H), 2.1 (m, 2H), 2.3 (m, 4H), 2.5 (m, 1H), 2.8 (m, 3H), 3.0 (s, 3H), 3.4 (m, 1H), 3.8 (m, 1H), 4.0 (dd, 1H), 5.1 (m, 2H), 5.4 (d, 1H, 5.7 (m, 1H), 7.2 (m, 10H), 7.6 (d, 2H), 7.9 (d, 2H); MS: 531.

### EXAMPLE 17

This Example illustrates the preparation of N′-phenylmethyl-N-[1-(3,3-diphenylpropyl)-4-piperidinyl]-N-allylurea (Compound No. 245 of Table II).

3-Phenylbutyraldehyde (0.2 g, 1.36 mmol) was added to a solution of N′-phenylmethyl-N-[piperidin-4-yl]-N-allylurea hydrochloride (370 mg, 1.36 mmol) in methanol (20 ml). After 15 mins sodium triacetoxyborohydride (430 mg, 2.0 mmol) was added portionwise over 15 mins and the reaction was left to stir for 16 h. Water (5 ml) was added to the mixture and the methanol was removed in vacuo. The solution was diluted with water (30 ml), and partitioned with EtOAc (2×40 ml). The organic fractions were combined and washed with brine (30 ml), dried (MgSO₄) and concentrated. The oil was dissolved in MeOH (5 ml) and then applied to an ISOLUTE™ SCX column (5 g) which was then washed with MeOH (30 ml) followed by a 1:4 mixture of aqueous ammonia and methanol (30 ml). Addition of ethereal HCl to the final wash, followed by evaporation gave the title compound as a gum (152 mg, 0.38 mmol); MS: 406.

### EXAMPLE 18

This Example illustrates the preparation of N-[1-(3-phenyl-3-[4fluorophenyl]-3-hydroxypropyl)-4-piperidinyl]-N-ethyl-4-methanesulfonylphenylacetamide (Compound No. 11 of Table III).

To a solution of N-[1-(3-[4-fluorophenyl]-3-oxopropyl)-4-piperidinyl]-N-ethyl-4-methanesulfonylphenylacetamide hydrochloride (470 mg, 0.92 mmol) in THF (40 mL) under an inert atmosphere was added phenylmagnesium bromide (10 mL, 1 M in THF) at room temperature. After stirring for 1 h saturated aqueous sodium bicarbonate solution was added and the resulting mixture was extracted with ethyl acetate. The organic phase was dried (MgSO₄) and concentrated. The title compound was obtained by silica column chromatography, eluting with 10% methanol in ethyl acetate yielding 120 mg. NMR (CDCl₃): 1.18 and 1.23 (t, 3H), 1.65 (m, 2H), 1.84 (m, 2H), 2.42 (m, 2H), 3.02 (s, 3H), 3.35 (m, 2H), 3.65 (m, 4H), 3.68 and 3.78 (s, 2H), 4.73 (t, 2H), 6.97 (m, 2H), 7.2-7.4 (m, 9H), 7.90 (d, 2H); MS: 553.

### EXAMPLE 19

This Example illustrates the preparation of N-[1-(3-phenyl-4-pentenyl)-4-piperidinyl]-N-methyl-4-fluorophenylacetamide (Compound No. 12 of Table III).

5-Bromo-3-phenylpent-1-ene (131 mg, 0.58 mmol), 4-(N-(4-fluorophenyl-acetamido)-N-methyl)aminopiperidine (73 mg, 0.29 mmol), potassium carbonate (120 mg, 0.87 mmol) and tetrabutylammonium iodide (5 mg) were stirred in DMF (3 ml). After 16 h, water was added and the mixture extracted with EtOAc (2×20 ml). The organics were combined and washed with water, dried (MgSO₄), concentrated and purified by Bond Elut chromatography (eluent DCM, followed by 2.5% EtOW/DCM and finally 5% EtOH/DCM) to afford the title compound as an oil (55 mg, 0.14 mmol); MS: 395.

### EXAMPLE 20

This Example illustrates the preparation of N-[1-(3-phenyl-3-azetidinylpropyl)-4-piperidinyl]-N-methyl-4-fluorophenylacetamide dihydrochloride (Compound No. 13 of Table III).

To a solution of N-[1-(3-phenyl-3-chloropropyl)-4-piperidinyl]-N-methyl-4-fluorophenylacetamide (120 mg, 0.3 mmol) in DCM (5 mL) was added azetidine (0.12 mL, 1.8 mmol) and the resulting mixture was stirred at room temperature for 18 h. The reaction mixture was washed with water, dried (MgSO₄) concentrated, and purified by Bond Elut chromatography (eluent 5% MeOH/DCM followed by 10% MeOH/DCM) to afford the title compound as an oil which was then treated with ethereal HCl to provide N-[1-(3-phenyl-3-azetidinylpropyl)-4-piperidinyl]-N-methyl-4-fluorophenylacetamide dihydrochloride as a white solid (35 mg, 24%); NMR (d6-DMSO, 373K): 1.5-1.65 (m, 2H), 1.85-2.1 (m, 4H), 2.55-2.9 (m, 8H), 3.1-3.2 (m, 1H), 3.25-3.35 (m, 1H), 3.6-3.75 (m, 5H), 4.1-4.2 (m, 2H), 7.0-7.1 (m, 2H), 7.2-7.3 (m, 2H), 7.35-7.5 (m, 5H); MS: 424.

### EXAMPLE 21

This Example illustrates the preparation of N-[1-(3-phenyl-3-[4-fluorophenyl]propyl)-4-piperidinyl]-N-ethyl-4-methanesulfonylphenylacetamide (Compound No. 15 of Table III).

To a solution of 4-(N-(4-fluorophenylacetamido)-N-methyl)aminopiperidine (143 mg, 1.74 mmol) in DMF (5 mL) was added 3-phenyl-3-(4-fluorophenyl)-1-bromopropane (Method V) (420 mg, 1.5 mmol) and K₂CO₃ (300 mg). The reaction was then stirred overnight and poured onto water (20 mL). Extracted into EtOAc, washed with water (20 mL), brine (20 mL), and dried over MgSO₄. The solvents were evaporated and the crude product was purified by Bond Elut chromatography (eluent 5% MeOH/DCM) to afford the title compound as a sticky gum, (148 mg, 20%); NMR: 1.65 (2H, m), 2.20 (1H, broad t), 3.2-2.6 (9H, m), 3.8-3.6 (6H, m), 4.10 (1H, m) and 7.4-7.2 (13H, m); MS: 463.

### EXAMPLE 22

This Example illustrates the preparation of N-[1-(3,3-di-[4-fluorophenyl]propyl)-4-piperidinyl]-N-ethyl-4-methanesulfonylphenylacetamide (Compound No. 16 of Table III).

To a DMF solution of 1-(3,3-di-(4-fluorophenyl)propyl)-4-(methylamino)piperidine (250 mg, 0.72 mmol, in 5 mL) was added 4-fluorophenylacetic acid (115 mg, 0.75 mmol), HATU (285 mg, 0.75 mmol), and DIPEA (130 μl). The reaction was stirred overnight and poured into water (20 mL). The organics were extracted into EtOAc (20 mL) and dried over MgSO₄. The desired product was then precipitated from the EtOAc by addition of 2M HCl in Et₂O, to afford a pale yellow gum (139 mg, 46%); NMR: 1.60 (2H, m), 2.20 (2H, m), 2.75 (3H, s), 3.3-3.7 (12H, m), 6.80 (2H, m) and 7.3-7.0 (10H, m); MS: 481.

### EXAMPLE 23

This Example illustrates the preparation of N-[1-(N,N-diphenyl-2-ethylamino)-4-piperidinyl]-N-allyl-4-methanesulfonylphenylacetamide (Compound No. 18 of Table III).

To a mixture of N-(4-piperidinyl)-N-allyl-4-methanesulfonylphenylacetamide (0.25 g, 0.74 mmol) and 4-methyl-2-pentanone (10 μL) was added potassium carbonate (0.31 g), potassium iodide (100 mg) and N-(2-bromoethyl)diphenylamine (0.21 g) and the resulting mixture was stirred and heated to reflux for 18 h. After cooling, water was added and the volatiles removed by evaporation. The residue was extracted three times with ethyl acetate and the combined extracts were dried and concentrated to give an oil which was purified by eluting through a silica gel column with 1% methanol in dichloromethane then 5% methanol in dichloromethane to give the title compound (73 mg); NMR: 1.5 (m, 4H), 2.1 (m, 2H), 2.5 (m, 2H), 3.1 (s, 3H), 3.8 (m, 7H), 3.9 (s, 2H), 5.1 (m, 2H), 5.8 (m, 1H), 6.9 (m, 6H), 7.2 (m, 4H), 7.4 (d, 2H), 7.8 (d, 2H); MS: 532.

### EXAMPLE 24

This Example illustrates the preparation of N-[1-(N-phenyl-N-[2-(4-hydroxyphenyl)ethylcarbonyl]-2-ethylamino)-4-piperidinyl]-N-ethyl-4-methanesulfonylphenylacetamide (Compound No. 20 of Table III).

To 3-(4-hydroxyphenyl)propanoic acid (0.1 mmol) was added DMF (5 μL) followed by oxalyl chloride (1 mL of a 0.1M solution in DCM, 0.1 mmol) and the resulting mixture was shaken at room temperature for 2 h. 100 μL Of this mixture was then added to 100 μL of a solution of N-[1-(N-phenyl-2-ethylamino)-4-piperidinyl]-N-ethyl-4-methanesulfonylphenylacetamide (230 mg, 0. mmol) and triethylamine (0.334 mL, 2.4 mmol) in DCM (12 mL). The resulting mixture left at room temperature for 20 h then water (250 μL) and DCM (250 μL) were added and the mixture was shaken. The aqueous phase was removed and the organic phase was concentrated giving the title compound which was characterised by LC-MS; MS: 591.

### EXAMPLE 25

This Example illustrates the preparation of N-[1-(3-phenyl-3-aminopropyl)-4-piperidinyl]-N-ethyl-4-methanesulfonylphenylacetamide dihydrochloride (Compound No. 23 of Table III).

To a solution of 3-phenyl-3-Bocaminopropanal (513 mg, 2.0 mmol) and N-(4-piperidinyl)-N-ethyl-4-methanesulfonylphenylacetamide (645 mg, 2.0 mmol) in methanol (15 mL) was added acetic acid (0.2 mL) and the resulting mixture was stirred at room temperature for 1 h. Sodium triacetoxyborohydride (844 mg, 4.0 mmol) was added and the mixture was stirred at room temperature for 18 h then evaporated. The residue was partitioned between DCM and water, and the organic phase was washed with brine, dried and concentrated. The residue was suspended in 4M HCl in dioxane (20 mL) and methanol (5 mL) was added. The resulting mixture was heated to reflux for 7 h, then cooled to room temperature and concentrated giving an oily residue which was purified by silica gel chromatography (eluent 5% MeOH/DCM then 10% MeOH/DCM) yielding the title compound as a solid (675 mg); NMR (d6 DMSO at 373K): 1.1 (t, 3H), 1.5 (m, 2H), 1.9 (m, 2H), 2.0 (m, 1H), 2.3 (m, 2H), 3.0 (m, 1H), 3.2 (m, 4H), 3.3 (q, 2H), 3.9 (s, 2H), 4.0 (m, 1H), 4.4 (m, 1H), 7.4 (m, 3H), 7.5 (m, 4H), 7.9 (m, 2H); MS: 458.

### EXAMPLE 26

This Example illustrates the preparation of N-[1-(3-phenyl-3-benzoylaminopropyl)-4-piperidinyl]-N-ethyl-4-methanesulfonylphenylacetamide (Compound No. 1 of Table IV).

A solution of benzoic acid (0.005 mmol) in NMP (50 μL) was added to a solution of HATU (0.01 mmol) and diisopropylethylamine (0.03 mmol) in NMP (100 μL). To the resulting mixture was added N-[1-(3-phenyl-3-aminopropyl)-4-piperidinyl]-N-ethyl-4-methanesulfonylphenylacetamide dihydrochloride (Example 25; 0.005 mmol) in NMP (100 μL). The mixture was left at room temperature for 18 h, then evaporated. The residue was partitioned between DCM (250 μL) and water (250 μL) and the phases separated. The organic phase was concentrated giving the title compound which was characterised by LC-MS; MS: 562.

### EXAMPLE 27

This Example illustrates the preparation of N-[1-(N-Phenyl-2-ethylamino)-4-piperidinyl]-N-ethyl-4-methanesulfonylphenylacetamide (Compound No. 24 of Table III).

To a mixture of N-(4-piperidinyl)-N-ethyl-4-methanesulfonylphenylacetamide (2.0 g, 6.2 mmol) and N-(2-chloroethyl)aniline hydrochloride (1.2 g, 6.2 mmol) (J. Med. Chem. 1965, 173) in 4-methyl-2-pentanone (15 mL) was added potassium carbonate (2.56 g, 18.6 mmol) and potassium iodide (150 mg, 0.9 mmol) and the resulting mixture stirred at reflux for 20 h. After cooling to room temperature the solid was removed by filtration and the filtrate concentrated. The residue was purified by Bond Elut chromatography (eluent 5% MeOH/DCM) to afford, after trituration with diethyl ether, the title compound as a white solid (1.30 g, 50%); NMR (d6 DMSO, 373K): 1.1 (t, 3H), 1.4 (m, 2H), 1.8 (m, 2H), 2.1 (m, 2H), 2.5 (m, 2H), 3.1 (m, 5H), 3.3 (q, 2H), 3.8 (s, 2H), 5.0 (m, 1H), 6.6 (m, 3H), 7.1 (dd, 2H), 7.5 (d, 2H), 7.8 (d, 2H); MS: 444.

Compound No. 25 of Table III was prepared according to the method of Example 27 using N-(4-pipenrdinyl)-N-ethyl-4-fluorophenylacetamide. NMR: 1.0 and 1.5 (t, 3H), 1.3 (m, 1H) 1.5 (m, 1H), 1.7 (m, 2H), 2.0 (m, 2H), 2.4 (m, 2H), 2.9 (m, 2H), 3.1 (m, 2H), 3.2 (m, 2H), 3.6 and 3.7 (s, 2H), 4.1 (m, 1H), 5.2 (br s, 1H), 6.5 (m, 3H), 7.0 (dd, 2H), 7.1 (dd, 2H), 7.2 (m, 2H); MS: 384.

### EXAMPLE 28

This Example illustrates the preparation of Compound No. 26 of Table III.

To a solution of N-[1-(3-phenyl]-3-oxopropyl)-4-piperidinyl]-N-ethyl-4-methanesulfonylphenylacetamide hydrochloride (5.00 g, 10.1 mmol) in methanol (150 mL) was added sodium borohydride (0.96 g, 25.4 mmol) portionwise. The resulting mixture was stirred at room temperature for 20 h. Water (10 mL) was added and the mixture was evaporated. The residue was purified by silica column chromatography (gradient elution from ethyl acetate to 50% ethyl acetate/MeOH) to give the title compound (3.92 g, 84%); NMR: (CDCl₃): 1.14 and 1.23 (t, 3H), 1.56 (m, 1H), 1.75 (m, 2H), 1.83 (m, 3H), 1.98 (m, 1H), 2.20 (m, 1H), 2.56 (m, 1H), 2.66 (m, 1H), 3.02 (s, 3H), 3.10 (m, 1H), 3.18 (m, 1H), 3.31 (q, 2H), 3.57 and 4.49 (m, 1H), 3.79 and 3.80 (s, 2H), 4.94 (m, 1H), 7.23 (m, 1H), 7.34 (m, 4H), 7.44 (d, 2H) and 7.90 (d, 2H); MS: 459.

### EXAMPLE 29

This Example illustrates the preparation of N-[1-(4,4-diphenyl-but-2-yl)-4-piperidinyl]-N-ethyl-4-methanesulfonylphenylacetamide hydrochloride (Compound No. 27 of Table III).

N-(4-Piperidinyl)-N-ethyl-4-methanesulfonylphenylacetamide (323 mg, 1 mmol) was dissolved in DCM (10 mL). Acetic acid (1 ml) and 4,4-diphenyl-2-butanone (384 mg, 1.5 mmol) was added followed by sodium triacetoxyborohydride (516 mg, 2.1 mmol). The reaction mixture was stirred at room temperature for 7 days. Water (10 ml) was added and the layers separated. The organic phase was washed with brine, dried (MgSO₄) and evaporated to dryness. The residue was purified by Bond Elut chromatography (eluent 5% MeOH/DCM). The resultant oily residue was dissolved in a small amount of DCM, 1M HCl in diethyl ether was added and the mixture concentrated to yield the title compound as a white solid (120 mg, 22%); NMR (d6-DMSO, 373K): 1.0-1.2 (m, 6H), 1.5-2.1 (m, 6H), 2.5-3.0 (m, 6H), 3.1 (s, 3H), 3.3 (q, 2H), 3.8 (s, 2Hs), 4.1 (t, 1H) 7.1 (m, 2H), 7.2-7.4 (m, 8H), 7.5 (d, 2H), 7.9 (d, 2H); MS: 533.

### EXAMPLE 30

This Example illustrates the preparation of N-[1-(4-phenyl-but-2-yl)-4-piperidinyl]-N-ethyl-4-methanesulfonylphenylacetamide (Compound No. 28 of Table III).

To a mixture of N-(4-piperidinyl)-N-ethyl-4-methanesulfonylphenylacetamide (324 mg, 1 mmol), 4-phenyl-2-butanone (0.22 ml, 1.5 mmol), sodium triacetoxyborohydride (318 mg, 1.5 mmol) and acetic acid (0.11 ml, 2 mmol) in DCM (8 ml) was added a little MgSO₄ and the resulting mixture heated to reflux for 48 h. The reaction mixture was eluted through a column of silica gel (isohexane then 89% DCM/10% MeOH/10% NH₄OH) yielding the title compound (60 mg); NMR (CDCl₃): 1.1 and 1.2 (t, 3H), 1.3 (t, 3H), 1.6 (br m, 2H), 1.8 (m, 1H), 2.0 (s, 2H), 2.1 (m, 2H), 2.6 (br m, 3H), 3.0 (s, 3H), 3.2 (br m, 2H), 3.3 (q, 2H), 3.8 (s, 2H), 4.5 (m, 1H), 7.2 (m, 3H), 7.3 (m, 2H), 7.4 (m, 2H) and 7.9 (m, 2H); MS: 457.

### EXAMPLE 31

This Example illustrates the preparation of N-[1-(3-[3-trifluoromethylphenyl]-butyl)-4-piperidinyl]-N-ethyl-4-methanesulfonylphenylacetamide (Compound No. 29 of Table III).

To a solution of N-(4-pipetidinyl)-N-ethyl-4-methanesulfonylphenylacetamide (680 mg, 2.1 mmol) in MeOH/DCM (10 ml, 1:1) was added 3-(3-trifluoromethylphenyl)butyraldehyde (Method BP) (500 mg, 2.3 mmol) and acetic acid (0.25 ml). The resulting mixture was stirred at room temperature for 30 min. then sodium triacetoxyborohydride (735 mg, 3.2 mmol) was added. The resulting mixture was stirred at room temperature for 2 h then quenched with water (5 ml) and concentrated to a third of the volume. The residual mixture was extracted with DCM and the organic extracts washed with saturated NaHCO₃ solution and brine and evaporated to give the title compound (260 mg); NMR (CDCl₃): 1.18 (t, 3H), 1.3 (t, 3H), 1.5 (m, 1H), 1.7 (m, 6H), 2.0 (m, 2H), 2.2 (m, 2H), 2.8 (m, 3H), 3.05 (s, 3H), 3.3 (m, 2H), 3.8 (d, 2H), 7.4 (m, 6H), 7.9 (d, 2H); NMR: 525.

Compound No. 30 of Table III: NMR (CDCl₃): 1.18 (t, 3H), 1.3 (t, 3H), 1.5 (m, 1H), 1.7 (m, 8H), 2.2 (m, 2H), 2.7 (m, 1H), 2.9 (m, 2H), 3.05 (s, 3H), 3.3 (q, 2H), 3.8 (d, 2H), 7.05 (d, 1H), 7.2 (m, 3H), 7.45 (m, 2H), 7.9 (d, 2H); MS: 491.

Compound No. 31 of Table III: NMR (CDCl₃): 1.18 (t, 3H), 1.3 (t, 3H), 1.5 (m, 1H), 1.7 (m, 8H), 2.2 (m, 2H), 2.7 (m, 1H), 2.9 (m, 2H), 3.05 (s, 3H), 3.3 (q, 2H), 3.8 (d, 2H), 7.2 (d, 3H), 7.3 (m, 2H), 7.45 (m, 2H), 7.9 (d, 2H); MS: 457.

Compound No. 32 of Table III: NMR (CDCl₃): 1.18 (t, 3H), 1.3 (t, 3H), 1.5 (m, 1H), 1.7 (m, 8H), 2.2 (m, 2H), 2.7 (m, 1H), 2.9(m, 2H), 3.05 (s, 3H), 3.3 (q, 2H), 3.8 (d, 2H), 7.0 (d, 1H) 7.35 (d, 1H), 7.45 (d, 2H), 7.9 (d, 2H); MS: 525.

### EXAMPLE 32

This Example illustrates the preparation of N-[1-(3,3-diphenylpropyl)-3-pyrrolidinyl]-N-ethyl-4-methanesulfonylphenylacetamide (Compound No. 33 of Table III).

To a solution of 4-methanesulfonylphenylacetic acid (1.01 g, 4.72 mmol) in DCM (20 ml) was added carbonyldiimidazole (765 mg, 4.72 mmol) and the resulting mixture stirred at room temperature for 2 h. A solution of 3-amino-1-(3,3-diphenylpropyl)pyrrolidine di-(trifluoroacetic acid) salt (Method BQ) (2.4 g, 4.72 mmol) and triethylamine (1.43 g, 11.4 mmol) in DCM (10 mL) was added and the resulting mixture stirred at room temperature for 2 h. The mixture was washed twice with water (50 ml), dried and evaporated. The residue was purified by silica column chromatography (eluent DCM then ethyl acetate) giving the title compound (1.6 g); NMR: 1.5 (m, 1H), 2-2.2 (m, 6H), 2.6 (m, 2H), 3.5 (s, 2H), 3.95 (t, 1H), 4.1(m, 2H), 7.1-7.3 (m 10H), 7.5(d, 2H), 7.8(d, 2H), 8.3 (d, 1H); MS: 477.

### EXAMPLE 33

This Example illustrates the preparation of N-[1-(3-[4-chlorophenyl]-3-[4-pyridyl]propyl)-4-piperidinyl]-N-ethyl-4-methanesulfonylphenylacetamide (Compound No. 34 of Table III).

N-(4-Piperidinyl)-N-ethyl-4-methanesulfonylphenylacetamide (480 mg, 1.47 mmol) was dissolved in DCM (40 ml). Acetic acid (6 ml) and 3-(4-chlorophenyl)-3-(4-pyridyl)propionaldehyde (Method BR) (2.2 mmol) was added and the mixture stirred at room temperature for 30 min. followed by the addition of sodium triacetoxyborohydride (340 mg, 1.6 mmol). The reaction mixture was stirred at room temperature for 2 h. The reaction mixture was eluted through a column of silica gel (ethyl acetate then 89% DCM/10% MeOH/1% NH₄OH) yielding the title compound (60 mg); NMR (CDCl₃): 1.1 and 1.3 (t, 3H), 1.5 (br m, 1H), 1.8 (m, 4H), 2.2 (m, 4H), 2.9 (m, 2H), 3.0 (s, 3H), 3.3 (q, 2H), 3.5 (br m, 1H), 3.8 (m, 2H), 4.0 (m, 1H), 4.4 (br m, 1H), 7.1 (m, 4H), 7.3 (m, 2H), 7.5 (m, 2H), 7.9 (m, 2H) and 8.5 (m, 2H); MS: 554.

| Compound | |
|---|---|
| No in | |
| Table III | ¹H NMR (CDCl₃) |
| 35 | 1.1 and 1.3 (t, 3H), 1.5 (m, 1H), 1.7 (br m, 4H), 2.0 (m, |
| | 1H), 2.2 (m, 3H), 2.4 (m, 1H), 2.9 (m, 2H), 3.0 (s, 3H), |
| | 3.3 (q, 2H), 3.8 (m, 2H), 4.1 (m, 1H), 4.4 (m, 1H), 7.1 |
| | (m, 2H), 7.2 (m, 4H), 7.4 (m, 2H), 7.6 (t, 1H), 7.9 (d, 2H) |
| | and 8.5 (m, 1H) |
| 36 | 1.1 and 1.2 (t, 3H), 1.5 (br m, 1H), 1.7 (b rm, 4H), 2.0 |
| | (m, 1H), 2.2 (m, 2H), 2.3 (m, 2H), 2.4 (m, 1H), 2.9 (m, |
| | 2H), 3.0 (s, 3H), 3.3 (q, 2H), 3.5 (m, 1H), 3.8 (m, 2H), |
| | 3.9 (t, 1H), 4.4 (m, 1H), 5.9 (s, 2H), 6.7 (s, 2H), 7.2 |
| | (m, 4H), 7.4 (m, 2H) and 7.9 (d, 2H) |
| 37 | 1.1 and 1.2 (t, 3H), 1.4 (m, 1H), 1.7 (m, 2H), 1.8 (m, 2H), |
| | 2.0 (br t, 1H), 2.2 (m, 2H), 2.4 (d, 1H), 2.9 (m, 2H), 3.0 |
| | (s, 3H), 3.3 (m, 2H), 3.5 (m, 1H), 3.8 (m, 2H), 3.9 (m, |
| | 1H), 4.4 (m, 1H), 7.2 (m, 9H), 7.4 (m, 2H) and 7.9 (d, 2H) |
| 38 | 1.1 and 1.2 (t, 3H), 1.7 (br m, 4H), 2.0 (m, 1H), 2.2 (m, |
| | 2H), 2.4 (m, 1H), 2.9 (m, 2H), 3.0 (s, 3H), 3.3 (m, 2H), |
| | 3.5 (m, 1H), 3.6 (m, 1H), 3.8 (m, 2H), 4.0 (m, 1H), 4.4 |
| | (m, 1H), 7.3 (m, 10H) and 7.9 (d, 2H) |
| 39 | 1.1 and 1.2 (t, 3H), 1.4 (m, 1H), 1.7 (m, 2H), 1.8 (m, 2H), |
| | 2.0 (br t, 1H), 2.2 (m, 3H), 2.9 (m, 2H), 3.0 (s, 3H), 3.3 |
| | (m, 2H), 3.5 (m, 1H), 3.6 and 4.5 (m, 1H), 3.8 (m, 5H), 3.9 |
| | (t, 1H), 6.8 (d, 2H), 7.2 (m, 7H), 7.4 (m, 2H) and 7.9 |
| | (d, 2H) |
| 40 | 1.1 and 1.2 (t, 3H), 1.5 (m, 1H), 1.7 (m, 2H), 1.8 (m, 2H), |
| | 2.2 (m, 3H), 2.4 (m, 1H), 2.9 (m, 2H), 3.0 (s, 3H), 3.3 (m, |
| | 2H), 3.5 (m, 1H), 3.8 (m, 2H), 4.0 (br t, 1H), 4.4 (m, 1H), |
| | 7.2 (m, 9H), 7.4 (m, 2H) and 7.9 (d, 2H) |
| 41 | 1.1 and 1.2 (t, 3H), 1.5 (m, 1H), 1.7 (m, 2H), 1.8 (m, 2H), |
| | 2.0 (br t, 1H), 2.2 (m, 3H), 2.3 (s, 3H), 2.9 (m, 2H), 3.0 |
| | (s, 3H), 3.3 (m, 2H), 3.5 (m, 1H), 3.6 and 4.4 (m, 1H), 3.8 |
| | (m, 2H), 3.9 (t, 1H), 7.1 (m, 5H), 7.2 (m, 4H), 7.4 (m, 2H) |
| | and 7.9 (d, 2H) |
| 42 | 1.1 and 1.3 (t, 3H), 1.5 (m, 1H), 1.7 (m, 4H), 2.0 (br t, |
| | 1H), 2.2 (m, 3H), 2.4 (m, 1H), 2.9 (m, 2H), 3.0 (s, 3H), |
| | 3.3 (m, 2H), 3.6 (br m, 2H), 3.8 (m, 2H), 4.0 (m, 1H), 4.4 |
| | (m, 1H), 7.3 (m, 11H) and 7.9 (d, 2H) |
| 43 | 1.1 and 1.3 (t, 3H), 1.5 (m, 2H), 1.7 (m, 4H), 1.9 (m, 2H), |
| | 2.2 (m, 2H), 2.9 (m, 1H), 3.0 (s, 3H), 3.1 (m, 1H), 3.4 (m, |
| | 2H), 3.8 (m, 2H), 4.0 (t, 1H), 4.4 (m, 1H), 7.0 (m, 4H), 7.2 |
| | (m, 4H), 7.4 (d, 2H) and 7.9 (d, 2H). |
| 44 | 1.6 (m, 4H), 2.0 (m, 2H), 2.2 (m, 4H), 2.9 (d, 2H), 3.0 (s, |
| | 3H), 3.7 and 3.8 (s, 2H), 3.9 (m, 3H), 4.5 (m, 1H), 5.1 and |
| | 5.3 (m, 2H), 5.8 (m, 1H), 6.9 (m, 4H), 7.1 (m, 4H), 7.4 (d, |
| | 2H) and 7.9 (d, 2H). |

Starting materials are commercially available, have been described in the literature or can be prepared by adaptation of literature methods. Examples of literature methods include: P. Richter, Ch. Garbe and G. Wagner, E. Ger. Pharmazie, 1974, 29(4), 256-262; C. Oniscu, D. Nicoara and G. Funieru, “4-(Ureidosulfonyl)phenylacetic acid and its ureide”, RO79-966646, (Romanian document); and M. A. Zahran, M. M. Ali, Y. A. Mohammed and A. A. Shehata, Int. J. Chem., 1993, 4(3), 61.

Method A

4-Methylamino-1-N-(3,3-diphenylpropyl)piperidine dihydrochloride

To a solution of 4-tert-butoxycarbonylamino-1-N-(3,3-diphenylpropyl)piperidine (Method I) (15.9 g, 40 mmol) in THF (300 ml) was added lithium aluminium hydride (60 ml, 1M solution in THF, 60 mmol) and the mixture was refluxed. After 5 h the reaction mixture was cooled and sodium hydroxide was added carefully. The resultant granular precipitate was filtered off and the filtrate partitioned between water and EtOAc. The organic layer was dried (MgSO₄) and concentrated to a half of the original volume. 1M HCl in diethyl ether was then added to give the title compound as a white solid (13.8 g, 37 mmol); MS: 310.

Method B

4-Methylamino-1-N-(3-R/S-phenylbutyl)piperidine dihydrochloride

To a solution of 4-tert-butoxycarbonylamino-1-N-(3-R/S-phenylbutyl)piperidine (Method J) (22 g, 66 mmol) in THF (500 μl) was added lithium aluminium hydride (100 ml, 1M solution in THF, 0.1 mol) and the mixture was refluxed. After 5 h the reaction mixture was cooled and 3M sodium hydroxide and water were added carefully. The resultant granular precipitate was filtered off and the filtrate partitioned between water and EtOAc. The organic layer was dried (MgSO₄) and concentrated to a half of the original volume. 1M HCl in diethyl ether was then added to give the title compound as a white solid (21 g, 66 mmol); NMR: 1.2 (d, 3H), 2.0 (m, 6H), 2.8 (m, 4H), 3.4 (m, 7H), 7.1 (m, 5H), 9.3 (br s, 1H); MS: 247.

Method C

4-Propargylamino-1-N-(3-R/S-phenylbutyl)piperidine

To a solution of 1-(3-R/S-phenylbutyl)-4-piperidone (Method K) (500 mg, 2.2 mmol) in MeOH (8 ml) and acetic acid (2 ml) was added propargylamine (0.18 ml, 2.6 mmol). After 45 mins, sodium cyanoborohydride (170 mg, 2.7 mmol) was added and the reaction mixture left to stir at ambient temperature. After 16 h EtOAc was added and the reaction mixture was partitioned with dilute brine. The organic layer was separated, dried (MgSO₄) and concentrated to give the title compound as an oil (330 mg, 1.2 mmol); MS: 271.

Method D

4-Allylamino-1-N-(3,3-diphenylpropyl)piperidine

To a solution of 1-(3,3-diphenylpropyl)-4-piperidone (Method L) (500 mg, 2.2 mmol) in MeOH (8 ml) and acetic acid (2 ml) was added allylamine (0.19 ml, 2.6 mmol). After 45 mins, sodium cyanoborohydride (135 mg, 2.2 mmol) was added and the reaction mixture left to stir at ambient temperature. After 16 h EtOAc was added and the reaction mixture was partitioned with dilute brine. The organic layer was separated, dried (MgSO₄) and concentrated to give the title compound as an oil (170 mg, 0.50 mmol); MS: 335.

Method E

4-Allylamino-1-N-(3-R/S-phenylbutyl)piperidine

To a solution of 1-(3-R/S-phenylbutyl)-4-piperidone (Method K) (500 mg, 2.2 mmol) in MeOH (8 ml) and acetic acid (2 ml) was added allylamine (0.19 ml, 2.6 mmol). After 45 mins, sodium cyanoborohydride (170 mg, 2.7 mmol) was added and the reaction mixture left to stir at ambient temperature. After 16 h EtOAc was added and the reaction mixture was partitioned with dilute brine. The organic layer was separated, dried (MgSO₄) and concentrated to give the title compound as an oil (180 mg, 0.66 mmol); MS: 273.

Method F

4-Piperidinyl-N-2-phenylethyl-2,4-difluorophenylurea.trifluoroacetic acid salt

To a solution of 1-tert-butyoxycarbonylpiperidin-4-yl-N-2-phenylethyl-2,4-difluorophenylurea (Method O) (300 mg, 0.65 mmol) in DCM (4 ml) was added trifluoroacetic acid (1 ml). After 2 h the reaction mixture was concentrated to give the title compound as an oil (0.31 g, 0.65 mmol); MS: 360.

Method G

4-Amino-1-(3,3-diphenylpropyl)piperidine

To a solution of 4-tert-butoxycarbonylamino-1-N-(3,3-diphenylpropyl)piperidine (Method I) (10 g, 25 mmol) in DCM (100 ml) was added trifluoroacetic acid (20 ml) dropwise. After 3 h, toluene was added and the reaction mixture was concentrated to give the di-trifluoroacetic acid salt of the title compound as an oil (9.7 g, 19 mmol); MS: 295.

Method H

4-Amino-1-(3-R/S-phenylbutyl)piperidine.ditrifluoroacetic acid salt

To a solution of 4-tert-butoxycarbonylamino-1-(3-R/S-phenylbutyl)piperidine (Method J) (13.1 g, 39.5 mmol) in DCM (150 ml) was added trifluoroacetic acid (30 ml) dropwise. After 15 h, toluene was added and the reaction mixture was concentrated to give the di-trifluoroacetic acid salt of the title compound as an oil (12.8 g, 27.8 mmol); MS: 233.

Method I

4-tert-Butoxycarbonylamino-1-N-(3,3-diphenylpropyl)piperidine

To a solution of 4-(Boc-amino) piperidine (10 g, 50 mmol) in acetonitrile (200 ml) was added 3,3-diphenylpropyl bromide (15.1 g, 55 mmol), tetrabutylammonium iodide (2 g, 5 mmol) and potassium carbonate (15 g, 100 mmol) and the mixture refluxed. After 5 h the reaction mixture was cooled and poured into water. The solution was partitioned with EtOAc and the organic layer dried (MgSO₄), concentrated and purified by column chromatography (toluene:EtOAc, 1:1 with 1% triethylamine) to give the title compound as an oil (15.9 g, 40 mmol); MS: 395.

Method J

4-tert-Butoxycarbonylamino-1-(3-R/S-phenylbutyl)piperidine

To a stirred solution of 4-(Boc-amino) piperidine (45 g, 0.225 mol) in methanol (160 ml) was added 3-R/S-phenylbutyraldehyde (36.5 ml, 0.25 mol) followed by acetic acid (115 ml). After 1 hour, sodium triacetoxyborohydride (71.5 g, 0.34 mol) was added portionwise over 30 mins [Caution: effervescence and exotherm]. After 15 h water (60 ml) was added and the total mixture was concentrated to remove the methanol. Water (250 ml) was added and the mixture was extracted with EtOAc (3×500 ml). The combined organics were washed with water, brine and dried (MgSO₄) to give the title compound as a white solid that was further recrystallised from DCM/EtOAc (54.1 g, 0.163 mol); m pt 220-221° C.; NMR: 1.2 (m, 3H), 1.4 (s, 9H), 1.7 (m, 2H), 2.0 (m, 6H), 2.8 (m, 4H), 3.3 (m, 2H), 7.0 (br s, 1H), 7.3 (m, 5H); MS: 333.

Method K

1-(3-R/S-phenylbutyl)-4-piperidone

A solution of 1-(3-R/S-phenylbutyl)-4-piperidone ethylene ketal (Method M) (6.45 g, 23 mmol) in 6M hydrochloric acid (80 ml) was heated to reflux. After 3 h the reaction mixture was cooled and the pH was adjusted to pH 10 by the addition of 1M NaOH. The mixture was extracted with DCM (3×30 mL) and the combined organics were dried (MgSO₄), concentrated and purified by flash column chromatography (DCM to 5% MeOH/DCM) to give the title compound as an oil (2.3 g, 10 mmol); NMR (CDCl₃): 1.2 (d, 3H), 1.6 (s, 1H), 1.8 (q, 2H), 2.2-2.5 (m, 5H), 2.7 (m, 3H), 2.8 (q, 1H) and 7.1-7.4 (m, 5H); MS: 232.

Method L

1-(3,3-Diphenylpropyl)-4-piperidone

The procedure described in Method K was repeated using 1-(3,3-diphenylpropyl)-4-piperidone ethylene ketal (Method N) (5.3 g, 16 mmol) in place of 1-(3-R/S-phenylbutyl)-4-piperidone ethylene ketal to give the title compound as an oil (4.6 g, 16 mmol); NMR (CDCl₃): 2.3 (m, 2H), 2.4 (m, 6H), 2.7 (m, 4H), 4.05 (q, 1H) and 7.1-7.4 (m, 10H).

Method M

1-(3-R/S-Phenylbutyl)-4-piperidone ethylene ketal

To a solution of 4-piperidone ethylene ketal (10 g, 70 mmol) in MeOH (100 ml) was added acetic acid (5 ml) and 3-R/S-phenylbutyraldehyde (11.4 ml, 77 mmol) and the reaction mixture left to stir at ambient temperature. After 1 h sodium triacetoxyborohydride (21 g, 99 mmol) was added portionwise. After a further 3 h water was added and the methanol was partially removed by evaporation; more water was added and the mixture extracted with EtOAc (×3). The combined organics were washed with water, brine, dried (MgSO₄) and concentrated to give the title compound as an oil (17.8 g, 65 mmol); MS: 276.

Method N

1-(3,3-Diphenylpropyl)-4-piperidone ethylene ketal

To a solution of 4-piperidone ethylene ketal (5 g, 35 mmol) in acetonitrile (50 ml) was added potassium carbonate (9.6 g, 70 mmol) followed by 3,3-diphenylpropylbromide (9.6 g, 35 mmol) and tetrabutylammonium hydrogensulphate (1 g). After 16 h water was added and the acetonitrile was partially removed by evaporation; the mixture was then extracted with EtOAc (×3). The combined organics were washed with water, brine, dried (MgSO₄), concentrated and purified by flash column chromatography (DCM to 8% MeOH/DCM) to give the title compound as an oil (5.3 g, 16 mmol); MS: 338.

Method O

1-tert-Butyoxyarbonylpiperidin-4-yl-N-2-phenylethyl-2,4-difluorophenylurea

To a solution of 4-(2-phenylethylamino)-1-tert-butoxycarbonylpiperidine (Method P) (0.61 g, 2 mmol) in DCM (30 ml) was added 2,4-difluorophenylisocyanate (0.21 ml, 2 mmol). After 3 h water was added and the reaction mixture stirred for 20 mins. The organic layer was then separated and the aqueous layer partitioned with DCM. The combined organic layers were washed with water, dried (MgSO₄), concentrated and columned (20% EtOAc/iso-hexane to 40% EtOAc/iso-hexane) to give the title compound as an oil (0.73 g, 1.6 mmol); MS:460.

Method P

4-(2-Phenylethylamino)-1-tert-butoxycarbonylpiperidine

To a solution of 1-tert-butoxycarbonylpiperid-4-one (10 g, 50 mmol) and 2-phemethylamine.hydrochloride (7.9 g, 50 mmol) in MeOH (250 ml) was added sodium cyanoborohydride (6.3 g, 100 mmol). After 1.5 h, water was added carefully and the MeOH was partially removed by evaporation. The mixture was extracted with DCM (×3); the organics were combined and washed with water, dried (MgSO₄), concentrated and purified by column chromatography (DCM to 5% MeOH/DCM) to give the title compound as an oil (13.4 g, 44 mmol); NMR (CDCl₃): 1.5 (m, 9H), 1.9 (d, 2H), 2.2 (t, 4H), 2.8 (t, 2H), 2.9 (m, 2H), 3.0 (m, 2H), 3.85 (m, 1H), 4.1 (m, 2H) and 7.2-7.4 (m, 5H).

Method R

4-(Cyclopropylmethyl)amino-1-(3-R/S-phenylbutyl)piperidine

To a solution of 1-(3-R/S-phenylbutyl)-4-piperidone (Method K) (500 mg, 2.2 mmol) in MeOH (8 ml) and acetic acid (2 ml) was added cyclopropyhmethylamine (0.2 ml, 2.6 mmol). After 45 mins, sodium cyanoborohydride (170 mg, 2.7 mmol) was added and the reaction mixture left to stir at ambient temperature. After 16 h EtOAc was added and the reaction mixture was partitioned with dilute brine. The organic layer was separated, dried (MgSO₄) and concentrated to give the title compound as an oil (230 mg, 1.2 mmol); MS: 287.

Method S

4-Fluorocinnamanic acid tert-butyl ester

To a suspension of 4-fluorocinnamanic acid (1.66 g, 10 mmol) in toluene (15 mL) heated to 80° C., was added dimethylformamide di-tert-butylacetal (8.2 g, 40 mmol) dropwise, and the reaction heated for a further 30 minutes. Upon cooling, the reaction was partitioned between toluene and water (15 mL), and washed with NaHCO₃ solution (2×10 mL), and brine (10 mL). The organic layer was dried, and concentrated. Purified on a Bond Elut column (eluent DCM) to afford the desired product as a colourless oil (1.25 g, 5.6 mmol); NMR (CDCl₃): 1.57 (9H, s), 6.28 (1 h, d), 7.07 (2H, t) and 7.50 (3H, m).

Method T

3-Phenyl-3-(4-fluorophenyl)propionic acid tert-butyl ester

To a −78° C. solution of 4-fluorocinnamanic acid tert-butyl ester (Method S) (0.9 g, 4 mmol) in THF was added dropwise a solution of phenyllithium in hexanes (4 mL of 1.5M solution, 6 mmol). The reaction was stirred for 1 h and then quenched with water and extracted into EtOAc, dried and purified by Bond Elut chromatography (50:50 DCM/iso-hexane) to afford the title compound, as a colourless oil (500 mg, 1.8 mmol); NMR (CDCl₃): 1.21 (9H, s), 2.87 (2H, d), 4.40 (1H, t), 6.90 (2H, t) and 7.15 (7H, m).

Method U

3-Phenyl-3-(4-fluorophenyl)-propan-1-ol

To a THF (10 mL) solution of 3-phenyl-3-(4-fluorophenyl)-propionic acid, tert-butyl ester (Method T) (495 mg, 1.65 mmol) was added LiAlH₄ in THF (2.5 ml of a 1.0M solution) and the reaction stirred at RT for 2 h. The reaction mixture was quenched cautiously with 2M aqueous NaOH, and the precipitate removed. The solution was then extracted with EtOAc, washed with water (20 mL) dried, MgSO₄, and evaporated to afford the title compound as a pale solid, (379 mg, 1.65 mmol); NMR (CDCl₃): 2.23 (2H, m), 3.65 (2H, t), 4.06 (1H, t), 6.90 (2H, m) and 7.20 (7H, m).

Method V

3-Phenyl-3-(4-fluorophenyl)-1-bromopropane

To a solution of 3-phenyl-3-(4-fluorophenyl)-propan-1-ol (Method U) (379 mg, 1.65 mmol) in DCM (5 mL), was added carbon tetrabromide (564 mg, 1.7 mmol), and triphenyl phosphine (445 mg, 1.7 mmol). The reaction was stirred overnight, and filtered through a pad of silica, then evaporated. The title product was obtained as a pale white solid by Bond Elut chromatography, eluent iso-hexane, (415 mg, 86%); NMR (CDCl₃): 2.43 (2H, m), 3.20 (2H, t), 4.16 (1H, t), 6.90 (2H, m) and 7.20 (7H, m).

Method W

4,4-Di-(4-fluorophenyl)-1-iodobutane

To a suspension of sodium iodide (1.5 g, 10 mmol) in acetone (100 mL) was added 4,4-di(4-fluorophenyl)-1-chlorobutane (2 g, 7 mmol), and refluxed for 5 h. The acetone was evaporated and the product was partitioned between water and EtOAc. The organic phase was dried (MgSO₄) and evaporated to give the title compound as a pale yellow oil, (3 g, 2:1 mixture of product to starting material); NMR (CDCl₃): 1.80 (2H, m), 2.20 (2H, m), 3.20 (1 ⅓H, t, CH₂I), 3.55 (⅔H, t, CH₂Cl), 3.90 (1H, t), 6.96 (4H, m) and 7.16 (4H, m).

Method X

4,4-Di-(4-fluorophenyl)-but-1-ene

The crude 4,4-di-(4-fluorophenyl)iodobutane (Method W) (3 g) was added to potassium tert-butoxide (1.3 g, 12 mmol) in THF (30 mL), and stirred overnight. The product was extracted into EtOAc and washed with water (100 mL). The organic phase was dried (MgSO₄) and evaporated to afford a yellow oil. This was purified by chromatography (silica, iso-hexane) to afford the desired product as a colourless oil. (1.4 g, 82%); NMR: 2.80 (2H, t), 4.00 (1H, t), 4.98 (1H, dd) 5.05 (1H, dd), 5.70 (1H, ddt), 7.00 (4H, m) and 7.20 (4H, m).

Method Y

3,3-Di-(4-fluorophenyl)propanal

A DCM solution of 4,4-di-(4-fluorophenyl)-but-1-ene (Method X) (1.4 g, 5.7 mmol, in 20 mL) was cooled to −78° C. and exposed to ozone until a pale blue colour persisted (about 20 min). The reaction was then purged with oxygen until the colour faded, and finally quenched with triphenylphosphine (1.49 g, 5.7 mmol). Upon warning to RT the reaction was washed with water, dried (MgSO₄) and concentrated. The residue was passed through a plug of silica to afford the title product as a colourless oil, (1.18 g, 100%); NMR (CDCl₃): 3.15 (2H, d), 4.60 (1H, t), 7.00 (4H, m), 7.18 (4H, m), 9.75 (1H, s).

Method Z

1-(3,3-Di-[4-fluorophenyl]propyl)-4-([tert-butoxycarbonyl]amino)piperidine

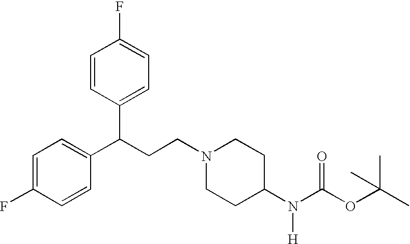

To a solution of 3,3-di-(4-fluorophenyl)propanal (Method Y) (1.18 g, 5.7 mmol), in dichloroethane (14 mL) and 4-Bocaminopiperidine (1.2 g, 6 mmol) was added acetic acid (0.3 mL), 3 Å molecular sieves (2 g), and sodium triacetoxyborohydride (1.27 g, 6 mmol), and the reaction mixture stirred for 5 h. The mixture was poured onto water and extracted into EtOAc (30 mL), dried and evaporated. The title product was obtained by purification by chromatography (silica, 5% MeOH/DCM) to give the product as a solid (1.7 g, 69%); MS: 431.

Method AA

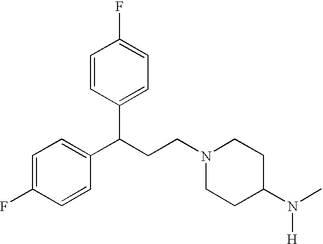

1-(3,3-Di-[⁴-fluorophenyl]propyl)-4-(methylamino)piperidine

To a solution of 1-(3,3-Di-[4-fluorophenyl]propyl)-4-([tert-butoxycarbonyl]amino)piperidine (Method Z) (1.7 g, 3.9 mmol) in THF (50 mL), was added LiAlH₄ solution (5 mL of a 1.0M solution in THF) dropwise (CARE gas evolution) and then the reaction was refluxed for 16 h. The reaction mixture was then cooled to RT and cautiously quenched with 2M NaOH, filtered to remove precipitate and partitioned between water and EtOAc. The organic layer was dried over MgSO₄ and evaporated. The crude product was purified by chromatography (silica, eluent 1:1, toluene:EtOAc with 0.5% isopropylamine) to afford the title compound as a yellow oil (500 mg, 37%); NMR: 2.2-1.0 (9H, m), 2.67 (1H, m), 3.4-3.2 (4H, m), 3.90-4.10 (2H, m), 4.35 (2H, m), 7.05 (4H, m) and 7.30 (4H, m); MS: 345.

Method AB

4-Ethylamino-1-N-(3,3-diphenylpropyl)piperidine

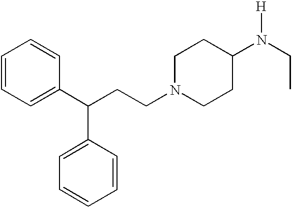

To a solution of 1-(3,3-diphenylpropyl)-4-piperidone (Method L) (2.2 g, 7.5 mmol) in DCM (30 ml) was added ethylamine (8.5 ml, 2M in THF, 17 mmol), sodium triacetoxyborohydride (1.6 g, 7.5 mmol) and 4 Å Molecular Sieves (10 rods). The reaction mixture left to stir at ambient temperature. After 16 h the mixture was filtered, washed with water, dried (NaSO₄) and concentrated to give the title compound as an oil (1.4 g, 4.35 mmol);

MS: 323.

Method AC

N[1-Phenylmethyl-piperidin-4-yl]-N-methyl-(4-fluorophenyl)acetamide

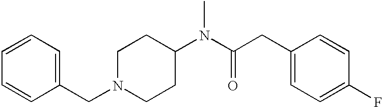

To a solution of 4-methylamino-1-N-(phenylmethyl)piperidine (2.95 g, 14.5 mmol) in DMF (25 ml) was added DIPEA (10 ml), 4-fluorophenylacetic acid (2.67 g, 17.3 mmol) and HATU (6.0 g, 16 mmol). After 16 h at RT water was added and the mixture was partitioned with EtOAc (×3). The organics were combined, washed with water and brine, dried (MgSO₄) and concentrated to give the title compound as a brown oil (4.90 g, 14.4 mmol); MS: 341. † 4-Methylamino-1-N-(phenylmethyl)piperidine is described in J. Med. Chem. 1999, 42, 4981-5001.

Method AD

4-(N-(4-Fluorophenylacetamido)-N-methyl)aminopiperidine

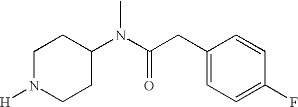

To a solution of N-[1-phenylmethyl-piperidin-4-yl]-N-methyl-(4-fluorophenyl)acetamide(Method AC) (4.90 g, 14.4 mmol) in EtOH (50 ml) was added 20% palladium hydroxide on carbon (1 g) followed by ammonium formate (5.18 g, 82 mmol). The reaction mixture was then refluxed until the evolution of gas ceased at which point it was filtered through Celite® and concentrated to give the title compound as an oil (2.86 g, 11.4 mmol); MS: 251.

Method AE

3-Phenylpent-4-enoic acid

Cinnamyl alcohol (5 g, 37 mmol), triethylorthoacetate (47 ml) and propionic acid (0.17 ml) were heated at 140° C. under a distillation head and condenser. After 1 h the reaction mixture was cooled and concentrated to give a pale yellow oil. This oil was dissolved in EtOH (15 ml) and water (15 ml) and NaOH (3.73 g, 93 mmol) was added and the mixture stirred at 80° C. After 16 h the mixture was heated to 100° C. for 2 h then allowed to cool. The reaction mixture was diluted with water (120 ml) and extracted with diethyl ether (2×150 ml). The aqueous layer was acidified with AcOH and then re-extracted with diethyl ether (3×150 ml). The organics were combined and dried (MgSO₄) and concentrated to give the desired product as a brown oil (5.52 g, 31 mmol); NMR: 2.65 (m, 2H), 3.75 (1, 1H), 4.95 (s, 1H), 5.05 (d, 1H), 5.95 (m, 1H), 7.2 (m, 5H), 12.1 (br s, 1H); MS: 177.

Method AF

3-Phenylpent-4-en-1-ol

To a solution of 3-phenylpent-4-enoic acid (Method AE) (2.0 g, 11.4 mmol) in THF (20 ml) at 0° C. was added lithium aluminium hydride (12.5 ml, 1M solution in THF) dropwise over 15 mins and the reaction mixture was allowed to warm to RT. After 64 h water (2.4 ml) was added followed by 2N NaOH (2.4 ml) then water (7.2 ml). The resulting gelatinous precipitate was filtered, washed with THF and concentrated. The residue was dissolved in DCM and washed with saturated sodium hydrogen carbonate (2×150 ml), dried (MgSO₄) and concentrated to give the title compound as a pale yellow oil (1.8 g, 1.1 mmol); NMR: 1.8 (m, 2H), 3.4 (m, 2H), 4.4 (t, 1H), 5.0 (m, 2H), 5.9 (m, 1H) and 7.2 (m, 5H).

Method AG

5-Bromo-3-phenylpent-1-ene

The procedure described in Method V was repeated except using 3-phenylpent-4-en-1-ol (1.75 g, 10.8 mmol), triphenylphosphine (3.12 g, 11.9 mmol), carbon tetrabromide (3.94 g, 11.9 mmol) and DCM (35 ml) to give the title compound as a colourless oil (2.02 g, 9 mmol); NMR: 2.2 (m, 2H), 3.4 (m, 3H), 5.1 (m, 2H), 5.95 (m, 1H) and 7.2 (m, 5H).

Method AH

N-[-(3-[4-Fluorophenyl]-3-oxopropyl)-4-piperidinyl]-N-ethyl-4-methanesulfonylphenylacetamide hydrochloride

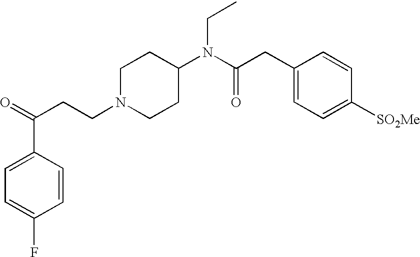

To a solution of N-4-piperidinyl-N-ethyl-4-methanesulfonylphenylacetamide (1.3 g, 4.0 mmol) in DMF (25 mL) was added DIPEA (2 mL, 11.5 mmol) and 3-chloro-4′-fluoropropiophenone (770 mg, 4.0 mmol). The resulting mixture was stirred at room temperature overnight then evaporated. The residue was heated to reflux with 5% methanol in ethyl acetate giving a white solid which was isolated (1.6 g, 80%). NMR: 1.00 and 1.16 (t, 3H), 1.75 (t, 2H), 2.23 (q, 2H), 3.10 (t, 2H), 3.18 (s, 3H), 3.30 (m, 2H), 3.35 and 3.64 (q, 2H), 3.56 (m, 2H), 3.82 and 3.93 (s, 2H), 4.15 and 4.28 (m, 1H), 7.40 (m, 2H), 7.50 (m, 2H),7.83 (m, 2H), 8.07 (m, 2H); MS: 475.

Method AI

N-(4-Piperidinyl)-N-ethyl-4-methanesulfonylphenylacetamide

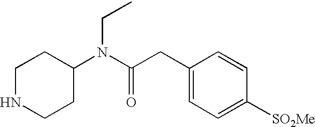

To a solution of N-(1-phenylmethyl-4-piperidinyl)-N-ethyl-4-methanesulfonylphenylacetamide (34 g, 82 mmol) in ethanol (600 mL) was added ammonium formate (40 g). The mixture was purged with argon and 30% Pd on carbon (4.2 g) was added. The resulting mixture was stirred at reflux for 4 h, then allowed to cool and filtered through diatomaceous earth. The filtrate was evaporated to give a thick oil which solidified on standing to yield the title compound (24.9 g, 94%); NMR: 1.02 and 1.15 (t, 3H), 1.4-1.6 (br m, 4H), 2.45 (m, 2H), 2.93 (br m, 2H), 3.18 (s, 3H), 3.20 and 3.32 (q, 2H), 3.72 and 4.18 (m, 1H), 3.80 and 3.87 (s, 2H), 7.50 (m, 2H), 7.85 (m, 2H); MS: 325 (MH+).

Method AJ

N-(1-Phenylmethyl-4-piperidinyl)-N-ethyl-4-methanesulfonylphenylacetamide

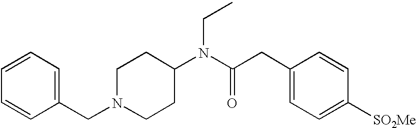

To a solution of 1-phenylmethyl-4-ethylaminopiperidine dihydrochloride (32.0 g, 110 mmol) in DCM (500 mL) was added N,N-diisopropylethylamine (60 mL) with stirring to ensure complete dissolution. 4-Methanesulfonylphenylacetic acid (25.0 g, 117 mmol), 4-Dimethylaminopyridine (4-DMAP) (2.0 g) and dicyclohexylcarbodiimide (DCCI) (25.0 g, 121 mmol) were added and the resulting mixture was stirred at room temperature for 20 h. The precipitate was removed by filtration and the resulting solution was washed successively with 2N aqueous HCl, water and 1N aqueous NaOH, dried (MgSO₄) and evaporated. The residue was purified by silica gel chromatography (eluent 10% MeOH/ethyl acetate) to afford the title compound (35 g, 76%); NMR: 1.00 and 1.14 (t, 3H), 1.45 and 1.70 (m, 2H), 1.95 (br m, 2H), 2.80 (br m, 2H), 3.18 (s, 3H), 3.20 and 3.33 (q, 2H), 3.45 (s, 2H), 3.80 and 3.87 (s, 2H), 3.70 and 4.10 (m, 1H), 7.2-7.3 (m, 5H), 7.48 (m, 2H), 7.82 (m, 2H); MS: 415 (MH+).

Method AK

1-Phenylmethyl-4-ethylaminopiperidine dihydrochloride

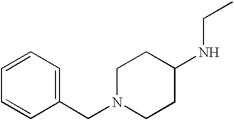

To a solution of 1-phenylmethyl-4-piperidone (25.0 g, 132 mmol) in THF (250 mL) was added ethylamine hydrochloride (12.0 g, 147 mmol) and methanol (50 mL) and the resulting mixture stirred at room temperature for 10 min. Sodium triacetoxyborohydride (40 g, 189 mmol) was added portionwise and the resulting mixture stirred at room temperature for 1 h. 2M Sodium hydroxide solution (250 mL) was added and the resulting mixture extracted with diethyl ether. The organic extracts were dried (K₂CO₃) and evaporated to give 1-phenylmethyl-4-ethylaminopiperidine as an oil. This was dissolved in ethanol (500 mL) and concentrated hydrochloric acid (20 mL) was added. The resulting crystals were collected, washed with diethyl ether and dried giving the title compound as a solid (38 g); NMR: (CDCl₃): 1.10 (t, 3H), 1.40 (m, 2H), 1.83 (m, 2H), 2.02 (m, 2H), 2.65 (q, 2H), 2.85 (m, 2H), 3.50 (s, 2H), 3.75 (m, 1H), 7.2-7.4 (m, 5H); MS: 219 (MH+).

Method AL

N-[1-(3-Phenyl-3-chloropropyl)-4-piperidinyl]-N-methyl-4-fluorophenylacetamide

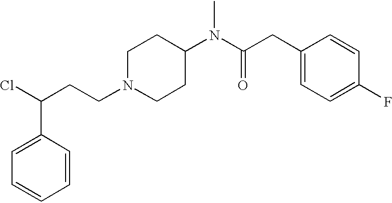

To a cooled (5° C.) solution of N-[1-(3-phenyl-3-hydroxypropyl)-4-piperidinyl]-N-methyl-4-fluorophenylacetamide (112 mg, 0.29 mmol) in DCM (5 mL) was added N,N-diisopropylethylanine (0.10 mL, 0.58 mmol) then methanesulfonyl chloride (0.03 mL, 0.35 mmol). The resulting mixture was stirred at ambient temperature for 18 h, then was concentrated. The residue was purified by Bond Elut chromatography (eluent DCM, followed by 5% MeOH/DCM) to afford the title compound as an oil (120 mg) which was characterised by LC-MS; MS: 403, 405.

Method AM

N-[1-(3-Phenyl-3-hydroxypropyl)piperidinyl]-N-methyl-4-fluorophenylacetamide

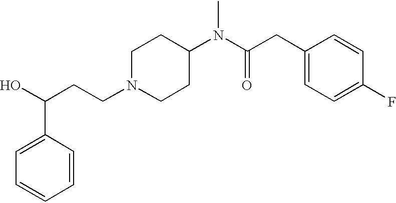

To a solution of N-[1-(3-phenyl-3-oxopropyl)-4-piperidinyl]-N-methyl-4-fluorophenylacetamide (300 mg, 0.78 mmol) in methanol (30 mL) was added sodium borohydride (120 mg) and the resulting mixture was stirred at room temperature for 2 h. Water (5 mL) was added and the mixture was concentrated. The residue was extracted with DCM and the organic extract was washed with water and brine, dried and concentrated to give the title compound (230 mg, 76%); NMR: 1.4 (m, 2H), 1.7 (m, 4H), 1.9 (m, 2H), 2.7 and 2.8 (s, 3H), 2.9 (m, 2H), 3.65 and 3.75 (s, 2H), 4.2 (m, 1H), 4.6 (m, 1H), 5.4 (br s, 1H), 7.1 (m, 2H), 7.2 (m, 3H), 7.3 (m, 4H); MS: 385.

Method AN

N-[1-(3-Phenyl-3-oxopropyl)-4-piperidinyl]-N-methyl-4-fluorophenylacetamide

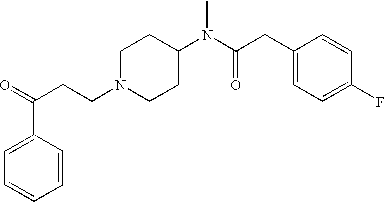

To a solution of N-(4-piperidinyl)-N-methyl-4-fluorophenylacetamide (250 mg, 1.0 mmol) in DMF (10 mL) was added 3-chloropropiophenone (168 mg, 1.0 mmol) and DIPEA (0.35 mL, 2.0 mmol). The resulting mixture was stirred at room temperature for 3 h. Water and DCM were added and the phases separated. The organic phase was washed with brine, dried and concentrated. The residue was purified by silica column chromatography (eluent 10% MeOH in DCM) yielding the title compound (305 mg); NMR: 1.3 (m, 2H), 1.6 (m, 2H), 2.0 (m, 2H), 2.6 (s, 3H), 2.7 (m, 2H), 2.9 (m, 2H), 3.1 (t, 2H), 3.7 (m, 2H), 4.2 (m, 1H), 7.1 (m, 2H), 7.2 (m, 2H), 7.4 (dd, 2H), 7.6 (t, 1H), 7.9 (d, 2H); MS: 383.

Method AO

N-(2-Bromoethyl)diphenylamine

To a cooled (5° C.) solution of N,N-diphenylbromoacetamide (1.4 g, 5.0 mmol) in THF (20 mL) was added borane methyl sulfide complex (26 mL, 1.0M) gradually. The reaction mixture was stirred at room temperature for 4 h. 10% Acetic acid in methanol (30 mL) was added and the resulting mixture was stirred for 20 h. The solvent was removed by evaporation and the residue was partitioned between ethyl acetate and water. The organic phase was dried and concentrated to give the title compound (1.0 g); NMR (CDCl3): 3.52 (t, 2H), 4.10 (t, 2H), 7.00 (m, 4H), 7.23 (m, 6H).

Method AP

N,N-Diphenylbromoacetamide

To a cooled (5° C.) solution of diphenylamine (2.0 g, 12 mmol) in DMF (15 mL) was added sodium hydride (520 mg, 60% dispersion) followed by bromoacetyl bromide (3.58 g) and the resulting mixture was stirred for 2 h. Water was added gradually, then the mixture was extracted three times with ethyl acetate. The combined organic extracts were washed three times with brine, dried (MgSO₄) and evaporated to yield the title compound (3.4 g, 99%); NMR (CDCl₃): 3.83 (S, 2H), 7.35 (m, 10H).

Method AQ

N-(4-Piperidinyl)-N-allyl-4-methanesulfonylphenylacetamide

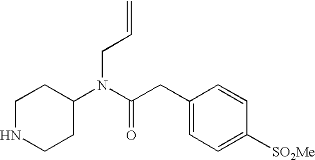

To a solution of N-(1-phenylmethyl-4-piperidinyl)-N-allyl-4-methanesulfonylphenylacetamide (4.40 g, 10.3 mmol) in DCM (30 mL) under an argon atmosphere and the mixture cooled in an ice-water bath. 1-Chloroethyl chloroformate (1.34 mL, 12.4 mmol) was added and the resulting mixture was stirred for 3 h while warming to room temperature. The mixture was evaporated and the residue dissolved in methanol (30 mL). The resulting mixture was refluxed for 1 h, allowed to cool and concentrated. The crude product was purified by silica column chromatography (eluent 5% EtOH/DCM then 15% EtOH/2% isopropylamine/DCM) to give the title compound (1.30 g); NMR: 1.50 (m, 4H), 2.50 (m, 2H), 2.95 (m, 2H), 3.20 (s, 3H), 3.74 and 3.91 (s, 1H), 3.80 and 3.95 (d, 1H), 4.29 (m, 1H), 5.00 and 5.05 (d, 1H), 5.20 (m, 1H), 5.73 and 5.89 (dddd, 1H), 7.44 and 7.49 (d, 2H), 7.85 (m, 2H).

Method AR

N-(1-Phenylmethyl-4-piperidinyl)-N-allyl-4-methanesulfonylphenylacetamide

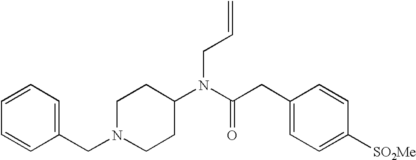

This was prepared by reacting 1-phenylmethyl-4-allylamine with 4-methanesulfonylphenylacetamide according to the procedure used for Method AJ; NMR (d6-DMSO, 373K): 1.65 (m, 2H), 1.88 (m, 2H), 2.39 (m, 2H), 3.05 (m, 2H), 3.09 (s, 3H), 3.75 (m, 4H), 3.93 (s, 2H), 4.08 (m, 1H), 5.15 (m, 2H), 5.82 (dddd, 1H), 7.30 (m, 5H), 7.45 (d, 2H), 7.80 (d, 2H).

Method AS

1-Phenylmethyl-4-allylamine

This was prepared by reacting 1-phenylmethyl-4-piperidone with allylamine according to the procedure used for Method AK; NMR (CDCl₃): 1.4 (m, 2H), 1.5 (m, 2H), 1.9 (m, 2H), 2.0 (dd, 2H), 2.5 (m, 1H), 2.8 (m, 2H), 3.3 (d, 2H), 3.5 (s, 3H), 5.1 (d, 1H), 5.2 (d, 1H), 5.9 (dddd, 1H), 7.3 (m, 5H); MS: 231 (MH+).

Method AT

N-4-Piperidinyl-N-ethyl-4-fluorophenylacetamide

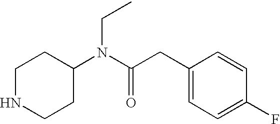

This was prepared by reacting N-(1-phenylmethyl-4-piperidinyl)-N-ethyl-4-fluorophenylacetamide according to the procedure used for Method AI; NMR: (formic acid salt): 0.97 and 1.10 (t, 3H), 1.46 and 1.62 (m, 2H), 1.8-2.0 (m, 2H), 2.78 (m, 2H), 3.1-3.3 (m, 4H), 3.65 and 3.74 (s, 2H), 3.97 and 4.22 (m, 1H), 7.08 (m, 2H), 7.25 (m, 2H), 8.42 (s, 1H); MS: 265.

Method AU

3-Phenyl -3-Boc-aminopropanal

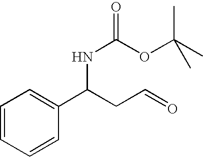

A solution of 3-phenyl-2-Boc-aminopropanol (700 mg, 2.78 mmol) in DCM (8 mL) was added to a stirred solution of Dess-Martin periodinane (1.30 g, 3.06 mmol) in DCM (5 mL) at room temperature followed by pyridine (0.3 mL). After stirring for 6 h at room temperature the mixture was partitioned between diethyl ether and saturated aqueous sodium bicarbonate solution containing sodium thiosulfate. The organic phase was washed with water and brine, dried and concentrated giving the title compound as a solid (790 mg); NMR: 1.4 (s, 9H), 2.8 (m, 2H),.5.1 (m, 1H), 7.3 (m, 5H), 8.6 (m, 1H), 9.6 (t, 1H).

Method AV

3-Phenyl-2-Boc-aminopropanol

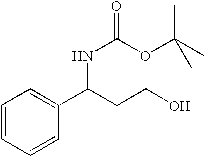

To a solution of 3-phenyl-3-Bocaminopropanoic acid (1.0 g, 3.78 mmol) in THF (10 mL) was added borane-THF complex (7.5 mL, 1.5M, 11.3 mmol) at 0° C. The resulting mixture was stirred with warming to room temperature for 5 h. 10% Acetic acid in methanol (20 mL) was added dropwise, the resulting mixture was concentrated and the residue partitioned between DCM and 1M aqueous HCl. The organic phase was washed with water and brine, dried (MgSO₄) and concentrated. The residue was purified by Bond Elut chromatography (eluent 5% MeOH/DCM) to afford the title compound (900 mg).

Method AW

3-Phenyl-3-Boc-aminopropanoic acid

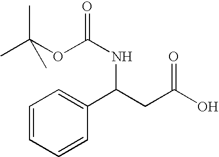

To a solution of DL-3-amino-3-phenylpropanoic acid (5 g, 30.2 mmol) in 2M aqueous sodium hydroxide (70 mL) was added a solution of di-tert-butyldicarbonate (8.56 g, 39.2 mmol) in THF (60 mL) and the resulting mixture stirred at room temperature for 48 h. Water (50 mL) was added and the mixture washed twice with ethyl acetate (50 mL). The aqueous phase was acidified to pH 3 with concentrated aqueous HCl, and the resulting mixture was extracted twice with ethyl acetate (60 mL). The combined organic extracts were dried (MgSO₄) and concentrated to give the title compound as a white solid (4.8 g); NMR: 1.4 (s, 9H), 2.7 (m, 2H), 4.8 (m, 1H), 7.3 (m, 5H), 7.5 (br d, 1H), 12.1 (br s, 1H); MS: 266.

Method AX

4-Cyclopropylamino-1-(3,3-diphenylpropyl)piperidine

This was prepared using a method similar to that used for 4-ethylamino-1-(3,3-diphenylpropyl)piperidine (Method AB). NMR: 0.0 (m, 2H), 0.2 (m, 2H); 1.1 (m, 2H), 1.55 (m, 2H), 1.7 (m, 2H), 1.9 (m, 5H), 2.5 (m, 2H), 3.7 (m, 1H), 6.9 (m, 2H), 7.1 (m, 8H); MS: 335.

Method AY

4-(2-Hydroxyethylamino)-1-(3,3-diphenylpropyl)piperidine

This was prepared using a method similar to that used for 4-ethylamino-1-(3,3-diphenylpropyl)piperidine. NMR: 1.2 (m, 2H), 1.7 (m, 2H), 1.9 (t, 2H), 2.1 (m, 4H), 2.3 (m, 1H), 2.7 (m, 2H), 3.1 (s, 3H), 3.4 (m, 1H), 3.95 (m, 1H), 7.1 (m, 2H), 7.3 (m, 8H); MS: 339.

Method AZ

4-(2-Fluoroethylamino)-1-(3,3-diphenylpropyl)piperidine

This was prepared using a method similar to that used for 4-ethylamino-1-(3,3-diphenylpropyl)piperidine; MS: 341.

Method BA

4-Chlorosulfonylphenylacetic acid.

Chlorosulfonic acid (10 ml, 148 mmol) was heated to 40° C. and phenyl acetic acid (5 g, 36.7 mmol) was added slowly. Stirred for two hours then cooled and carefully poured onto ice (50 g). The filtrate was cooled by filtration and dried under vacuum to afford the title compound as a pale cream solid. (7.9 g, 92%); NMR (CDCl₃), 3.80 (2H, s), 7.68 (2H, d), 8.00 (2H, d); MS: ES−233, ES+189.

Method BB

4-Fluorosulfonylphenylacetic acid.

18-Crown-6 (63 mg, 1 mol%) was added to a solution of 4-chlorosulfonylphenylacetic acid (5 g, 24 mmol) and KF (2.78 g, 48 mmol) in MeCN (5 mL) and stirred for 4 h. The product was then drowned out by the addition of water (100 mL) and collected by filtration to afford desired product (4.78 g, 97%); NMR (CDCl₃): 3.80 (2H, s), 7.68 (2H, d), 8.00 (2H, d); MS: 187.

Method BC

N-[1-(3,3-Diphenylpropyl)-4-piperidinyl]-N-methyl-4-fluorosulfonylphenylacetamide

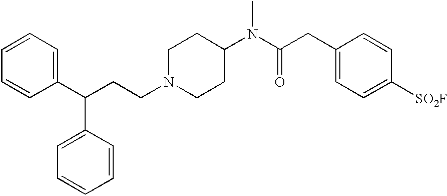

To a solution of HATU (836 mg, 2.2 mmol), 4-fluorosulfonylphenylacetic acid (409 mg, 2.2 mmol), 1-(3,3-diphenylpropyl)-4-methylaminopiperidine (618 mg, 2 mmol) in DMF (10 mL) was added DIPEA (0.4 mL) and stirred over night. Poured onto water and extracted into ethyl acetate (50 mL). Washed (brine 100 mL) and dried over MgSO₄, and evaporated to afford a pale yellow solid. Trituration with ethyl acetate/hexane (50:50) afforded the title product as a pale yellow solid (577 mg, 57%); NMR: 1.80 (2H, m), 2.00 (2H, m), 2.40 (2H, m), 2.80-3.20 (6H, m), 3.27 (3H, s), 3.45 (2H, m), 3.92 (1H, m), 4.46 (1H, m) 7.27 (8H, m), 7.60 (2H, t), 8.04 (2H, d); MS: 509.

Method BD

N-[1-(3,3-diphenylpropyl)-4-piperidinyl]-N-methyl-4-methoxycarbonylphenylacetamide

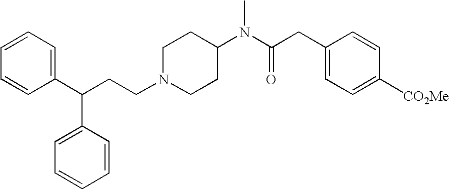

Solid HATU (2.55 g; 6.7 mmol) followed by DIPEA (1.22 ml; 6.7 mmol) was added at room temperature to a solution of 4-methoxycarbonylphenylacetic acid (1.3 g; 6.7 mmol) in DMF (10 ml). After 5 minutes, 4-methylamino-1-(3,3-diphenylpropyl)piperidine (2.1 g; 6.7 mmol) was added and stirring continued overnight at ambient temp. The mixture was then partitioned between water (10 ml) and ethyl acetate (10 ml). The organic layer was separated, washed with water (1 ml) and dried over Na₂SO₄ and evaporated to give an oil. Purification was by Bond Elut, eluting with a stepped gradient from DCM to 5% methanol in DCM yielding the title compound (2.47 g, 77%); MS: 485 (MH⁺).

Method BE

4-tert-Butoxycarbonylamino-1-(3-R-phenyl-1-butanoic amide)piperidine

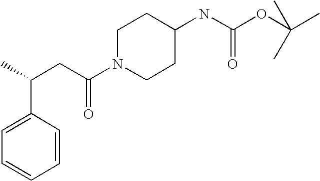

To a solution of 4-Boc-amino piperidine (2.46 g, 12.3 mmol) in DMF (30 mL) was added HATU (4.67 g, 12.3 mmol) and 3-R-phenyl-1-butanoic acid(2 g, 12.2 mmol) and DIPEA (2.12 mL). Stirred over night then poured into water and extracted into ethyl acetate. The organic extracts were dried over MgSO₄ and evaporated to afford the title compound as a white solid, (4.03 g, 94%); NMR: 1.20 (6H, m), 1.38 (9H, s), 1.65 (2H, m), 2.60 (2H, m), 3.00 (1H, m), 3.15 (1H, q), 3.40 (1H, m), 3.80 (1H, d, broad), 4.20 (1H, m), 6.80 (1H, m), 7.18 (1H, m), 7.24 (4H, m) MS: 347, 291 (−BOC).

Method BF

4-Amino-1-(3-R-phenyl-1-butanoic amide)piperidine hydrochloride

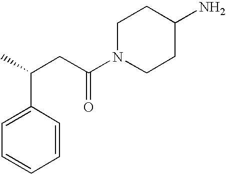

To a solution of acetyl chloride (5 mL) in methanol (20 mL) was 4-Boc-amino-1-(3-R-phenyl-1-butanoic amide)piperidine (1 g, 3 mmol) and stirred for one hour. The solvents were then evaporated to afford the title compound as a white solid. (929 mg, 100% for HCl salt); NMR: 1.20 (3H, d), 1.35 (2H, m), 1.41 (1H, m), 1.89 (2H, m), 2.80-3.20 (5H, m), 3.90 (1H, d), 4.30 (1H, d), 7.10 (1H, m), 7.20 (4H, m); MS: 247.

Method BG

4-Amino-1-(3-R-phenylbutyl)piperidine

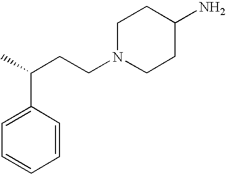

To a solution of 4-amino-1-(3-R-phenyl-1-butanoic amide)piperidine(1 g, 3 mmol) in THF (20 mL) was added a solution of LiAlH₄ in THF (10 mL of 1.0M solution) and the mixture was refluxed for 5 hours. The mixture was cooled, quenched with aqueous sodium hydroxide, filtered and the filtrate partitioned between water and ethyl acetate. The combined organic phase was dried, MgSO₄, and evaporated to afford the title compound as a white solid. (610 mg, 87%); NMR: 1.20 (4H, m), 1.60 (4H, m), 1.89 (2H, m), 2.10 (2H, m), 2.43 (1H, m), 2.70 (4H, m), 7.10 (3H, m), 7.20 (2H, m); MS: 233.

Method BH

4-tert-Butoxycarbonylamino-1-(3-S-phenyl-1-butanoic amide)piperidine

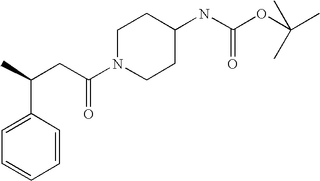

To a solution of 4-Boc-amino piperidine (2.46 g, 12.3 mmol) in DMF (30 mL) was added HATU (4.67 g, 12.3 mmol) and 3-S-phenyl-1-butanoic acid (2 g, 12.2 mmol) and DIPEA (2.12 mL). Stirred over night then poured into water and extracted into ethyl acetate. Dried over MgSO₄ and evaporated to afford the title compound as a white solid, (4.17 g, 99%); NMR: 1.20 (6H, m), 1.38 (9H, s), 1.65 (2H, m), 2.60 (2H, m), 3.00 (1H, m), 3.15 (1H, q), 3.40 (1H, m), 3.80 (1H, d, broad), 4.20 (1H, m), 6.80 (1H, m), 7.18 (1H, m), 7.24 (4H, m); MS: 347, 291 (−BOC).

Method BI

4-Amino-1-(3-S-phenyl-1-butanoic amide)piperidine hydrochloride

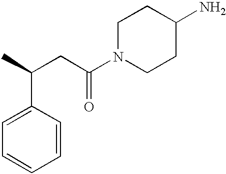

To a solution of acetyl chloride (5 mL) in methanol (20 mL) was added 4-Boc-amino-1-(3-S-phenyl-1-butanoic amide)piperidine(1 g, 3 mmol) and stirred for one hour. The solvents were then evaporated to afford the title compound as a white solid. (930 mg, 100% for HCl salt); NMR: 1.20 (3H, d), 1.35 (2H, m), 1. 41 (1H, m), 1.89 (2H, m), 2.80-3.20 (5H, m), 3.90 (1H, d), 4.30 (1H, d), 7.10 (1H, m), 7.20 (4H, m); MS: 247.

Method BJ

4-Amino-1-(3-S-phenylbutyl)piperidine

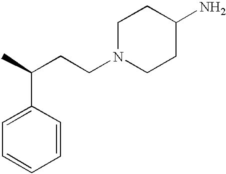

To a solution of 4-amino-1-(3-S-phenyl-1-butanoic amide)piperidine(1 g, 3 mmol) in THF (20 mL) was added a solution of LiAlH₄ in THF (10 mL of 1.0M soln) and the mixture was refluxed for 5 hours. The mixture was cooled, quenched with aqueous sodium hydroxide, filtered and the filtrate partitioned between water and ethyl acetate. The combined organic phase was dried, MgSO₄, and evaporated to afford the title compound as a white solid. (680 mg, 97%); NMR: 1.20 (4H, m), 1.60 (4H, m), 1.89 (2H, m), 2.10 (2H, m), 2.43 (1H, m), 2.70 (4H, m), 7.10 (3H, m), 7.20 (2H, m); MS: 233.

Method BK

N′-Phenylmethyl-N-(4-piperidinyl)-N-allylurea hydrochloride

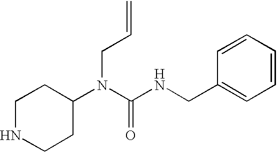

Acetyl chloride (5.5 mL) was added to methanol (20 mL) at 0° C. and the mixture stirred for 10 minutes before addition of a solution of N′-phenylmethyl-N-(1-tert-butyloxycarbonyl-4-piperidinyl)-N-allylurea (1.54 g, 4.17 mmol) in methanol (1 mL). The resulting mixture was stirred at 0° C. for 1 h and at room temperature for 1 h. Evaporation afforded the title compound as a solid (0.96 g); NMR: 1.60 (br d, 2H), 1.93 (m, 2H), 2.80 (m, 22H), 3.10 (m, 2H), 3.79 (d, 2H), 4.21 (m, 3H), 5.10 (d, 1H), 5.18 (dd, 1H), 5.80 (ddt, 1H), 7.20 (m, 5H), 9.21 (br s, 2H); MS: 274.

Method BL

N′Phenylmethyl-N-(1-tert-butoxycarbonyl-4-piperidinyl)-N-allylurea

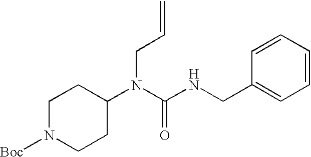

To a stirred solution of 1-tert-butoxycarbonyl-4-allylaminopiperidine (1.0 g, 4.17 mmol) in DCM (20 mL) was added benzylisocyanate (0.52 mL, 4.2 mmol) and the resulting mixture was stirred at room temperature for 20 h. Water was added and the mixture evaporated to yield the title compound (1.54 g, 99%); NMR 1.39 (s, 9H), 1.50 (m, 4H), 2.70 (m, 2H), 3.79 (d, 2H), 4.0 (mn, 3H), 4.21 (d, 2H), 5.10 (d, 1H), 5.18 (dd, 1H), 5.90 (ddt, 1H), 6.62 (t, 1), 7.20 (m, 5H); MS: 274 (MH⁺−BOC).

Method BM

1-tert-Butoxycarbonyl-4-allylaminopiperidine

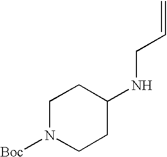

To a solution of 1-tert-butoxycarbonyl-4-piperidone (10.0 g, 50 mmol) in 1,2-dichloroethane (140 mL) was added allylamine (3.4 g, 60 mmol), acetic acid (3.0 mL) and 3 Å molecular sieves (20 g). The resulting mixture was stirred at room temperature for 45 min. Sodium triacetoxyborohydride (16.2 g, 76 mmol) was added and stirring was continued for a further 4 h. The reaction was quenched with water and extracted twice with ethyl acetate. The organic extracts were washed with sodium bicarbonate solution, combined, dried (MgSO₄) and concentrated to afford the title compound as an oil (11.5 g, 96%); NMR (CDCl₃): 1.21 (m, 2H), 1.40 (s, 9H), 1.60 (br s, 1H), 1.81 (d, 2H), 2.63 (m, 1H), 2.80 (t, 2H), 3.29 (t, 2H), 4.05 (d, 2H), 5.10 (d, 1H), 5.18 (dd, 1H), 5.90 (ddt, 1H).

Method BN

N-(1-Phenylmethyl-4-piperidinyl-N-ethyl-4-fluorophenylacetamide

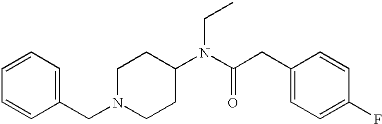

This was prepared by reacting 1-phenylmethyl-4-ethylaminopiperidine dihydrochloride with 4-fluorophenylacetic acid according to the procedure used for Method AJ; NMR (CDCl3): 1.13 and 1.19 (t, 3H), 1.35 and 1.85 (m, 2H), 1.74 and 2.08 (m, 2H), 2.90 (br m, 2H), 3.30 (m, 2H), 3.46 (s, 2H), 3.66 (s, 2H), 3.55 and 4.42 (m, 1H), 7.00 (m, 2H), 7.2-7.3 (m, 7H); MS: 355.

Method BO

N-[1-(3-phenyl]-3-oxopropyl)-4-piperidinyl]-N-ethyl-4-methanesulfonylphenylacetamide hydrochloride

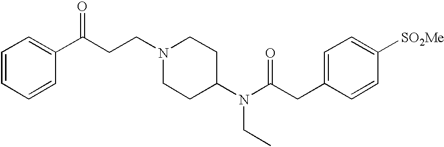

To a solution of N-(4-piperidinyl)-N-ethyl-4-methanesulfonylphenylacetamide (Method AI) (14.8 g, 45.8 mmol) and DIPEA (24mL, 137 mmol) in DMF (250 mL) was added 3-chloropropiophenone (7.3 g, 43.5 mmol). The resulting mixture was stirred at room temperature for 20 h. The mixture was evaporated and the residue triturated with 5% MeOH/EtOAc to give a solid which was collected by filtration and washed with EtOAc affording the title compound (16.9 g, 75%); NMR (DMSO at 373K): 1.14 (t, 3H), 1.77 (m, 2H), 2.34 (m, 2H), 3.11 (m, 2H), 3.15 (s, 3H), 3.45-3.60 (m, 6H), 3.65 (t, 2H), 3.93 (s, 2H), 4.25 (br m, 1H), 7.53 (m, 4H), 7.65 (m, 1H), 7.84 (d, 2H) and 7.98 (d, 2H); MS: 457.

Method BP

3-(3-Trifluoromethylphenyl)butyraldehyde

Step 1: (E)-Ethyl 3-(3-trifluoromethylphenyl)-2-butenoate

To a solution of triethyl phophonoacetate (1.98 ml, 10 mmol) in THF at 0° C. was added lithium bis(trimethylsilyl)amide (12 ml 1M in THF, 12 mmol) and the resulting mixture stirred. for 10 min. 3′-Trifluoromethylacetophenone (1.52 ml, 10 mmol) was added and the resulting mixture was stirred whilst allowing to warm to room temperature over 1 h. The mixture was evaporated and the residue partitioned between water and ethyl acetate, the organic phase was washed with brine, dried (MgSO₄) and evaporated. The residue was purified by Bond Elut chromatography (eluent isohexane then 1:1 ethyl acetate/isohexane) affording the sub-titled compound (1.4 g); NMR (CDCl₃): 1.3 (t, 3H), 2.6 (s, 3H), 4.2 (q, 2H), 6.15 (s, 1H), 7.15 (m, 1H), 7.6 (m, 2H), 7.7 (s, 1H).

Step 2: Ethyl 3-(3-trifluoromethylphenyl)butanoate

To a solution of (E)-ethyl 3-(3-trifluoromethylphenyl)-2-butenoate (Step 1) (1.4 g) in ethyl acetate (50 ml) was added 10% Pd/C (140 mg) and the resulting mixture was stirred under an atmosphere of hydrogen for 18 h. The mixture was filtered through Celite® and the filtrate evaporated to give the sub-titled compound (1.33 g); NMR (CDCl₃): 1.2 (t, 3H), 1.35 (d, 3H), 2.6 (m, 2H), 3.4 (m, 1H), 4.1 (q, 2H), 7.4 (m, 4H).

Step 3: 3-(3-Trifluoromethylphenyl)butanol

To a solution of ethyl 3-(3-trifluoromethylphenyl)butanoate (Step 2) (1.35 g, 5.2 mmol) in THF (15 ml) at 0° C. was added lithium aluminium hydride (5.2 ml, 1M in THF, 5.2 mmol) and the resulting mixture was stirred for 5 min. Ethyl acetate (10 mL) was added followed by water (0.2 ml) then 6M NaOH solution (0.2 ml) then water (2 ml) and the resulting mixture stirred at room temperature for 5 min. before filtration through Celite®. The filtrate was dried (MgSO₄) and evaporated giving the sub-titled compound (1.1 g); NMR (CDCl₃): 1.3 (d, 3H), 1.9 (m, 2H), 3.0 (m, 1H), 3.6 (m, 2H), 7.4 (m, 4H).

Step 4: 3-(3-Trifluoromethylphenyl)butyraldehyde

To a stirred solution of 3-(3-trifluoromethylphenyl)butanol (Step 3) (1.1 g, 5.05 mmol) in DCM (10 mL) was added Dess-Martin periodinane (2.36 g, 5.56 mmol) and the resulting mixture stirred at room temperature for 10 min. The mixture was washed three times with 2M NaOH solution (20 ml), then with brine (20 ml), dried (MgSO₄) and evaporated giving the title compound (1 g, 92%); NMR (CDCl₃): 1.34 (d, 3H), 2.75 (m, 2H), 3.43 (m, 1H), 7.46 (m, 4H), 9.73 (s, 1H).

The same sequence of reactions was used to prepare 3-(3-chlorophenyl)butyraldehyde and 3-(3,4-dichlorophenyl)butyraldehyde except that platinum (IV) oxide was used as catalyst in the reduction of (E)-ethyl 3-(3-chlorophenyl)-2-butenoate and (E)-ethyl 3-(3,4-dichlorophenyl)-2-butenoate to ethyl 3-(3-chlorophenyl)butanoate and ethyl 3-(3,4-dichlorophenyl)butanoate respectively.

Method BQ

3-Amino-1-(3,3-diphenylpropyl)pyrrolidine di-(trifluoroacetic acid) salt

Step 1: 3-Boc-amino-1-(3,3-diphenylpropyl)pyrrolidine

To a mixture of 3-boc-aminopyrrolidine (1 g, 5.4 mmol) and 3,3-diphenylpropionaldehyde (1.1 g, 5.4 mmol) in DCM (20 ml) and MeOH (5 ml) was added acetic acid (0.1 ml) and the resulting mixture stirred at room temperature for 1 h. Sodium triacetoxyborohydride (5.4 mmol) was added and the mixture stirred for 18 h. The reaction mixture was washed twice with water (10 ml), dried and evaporated giving the sub-titled compound (2.1 g); MS: 381.

Step 2: 3-Amino-1-(3,3-diphenylpropyl)pyrrolidine di-(trifluoroacetic acid) salt

3-Boc-amino-1-(3,3-diphenylpropyl)pyrrolidine (Step 1) (2.1 g) was dissolved in trifluoroacetic acid (10 mL) and the resulting mixture was stirred at room temperature for 2 h then evaporated giving the title compound (2.3 g).

Method BR

3-(4-Chlorophenyl)-3-(4-pyridyl)propionaldehyde

Step 1: 3-(4-Chlorophenyl)-3-(4-pyridyl)prop-1-ene

To a solution of 4-(4-chlorobenzyl)pyridine (1 g, 4.9 mmol) in THF was added n-butyl lithium (3.4 ml of 1.6M solution, 5.4 mmol) dropwise at room temperature. After stirring for 15 min. the mixture was cooled to −78° C. and allyl bromide (0.65 g, 5.4 mmol) was added dropwise. The reaction mixture was stirred while warming to room temperature over 18 h. The mixture was purified by Bond Elut chromatography (eluent isohexane then diethyl ether) giving the sub-titled compound as an oil (0.54 g); NMR (CDCl₃): 2.8 (t, 2H), 4.0 (t, 1H), 5.0 (m, 2H), 5.7 (m, 1H), 7.1 (m, 4H), 7.3 (m, 2H) and 8.5 (m, 2H); MS: 244.

Step 2: 3-(4-Chlorophenyl)-3-(4-pyridyl)propionaldehyde

3-(4-Chlorophenyl)-3-(4-pyridyl)prop-1-ene (Step 1) (0.54 g, 2.2 mmol) was dissolved in MeOH (30 ml) and the solution cooled to −78° C. Ozone was bubbled through until a blue colour persisted (20 min.). The mixture was purged with oxygen and dimethyl sulphide (0.33 ml) was added. The mixture was stirred for 1 h while warming to room temperature, then evaporated and the crude product used directly in the next reaction.

The same sequence of two reactions was used to prepare 3-(4-chlorophenyl)-3-(2-pyridyl)propionaldehyde.

Method BS

3-(1,3-Benzodioxol-5-yl)-3-phenylpropionaldehyde

Step 1: (E)-tert-Butyl 3-(1,3-benzodioxol-5-yl)propenonate

A solution of 3,4-methylenedioxycinnamic acid (0.77 g, 4 mmol) in toluene (10 ml) was heated with stirring to 80° C. and N,N-dimethylformamide di-tert-butyl acetal (3.83 ml, 16 mmol) was added dropwise. The resulting mixture was stirred at 80° C. for 2 h then cooled to room temperature. The mixture was washed with water and brine, dried (Na₂SO₄) and evaporated. The residue was purified by Bond Elut chromatography (eluent iso-hexane then DCM) giving the sub-titled compound as a solid (0.48 g).

Step 2: tert-Butyl 3-(1,3-benzodioxol-5-yl)-3-phenylpropionate

To a −78° C. solution of (E)-tert-butyl 3-(1,3-benzodioxol-5-yl)propenonate (Step 1) (2.4 mmol) in THF (5 ml) was added phenyl lithium (2 ml of 1.8M solution, 3.6 mmol) dropwise and the resulting mixture stirred at −78° C. for 2 h. Water (5 ml) was added and the mixture allowed to warm to room temperature over 18 h. The mixture was extracted with ethyl acetate, the organic phase was concentrated and the residue purified by Bond Elut chromatography (eluent iso-hexane then DCM) giving the sub-titled compound as an oil (0.51 g).

Step 3: 3-(1,3-Benzodioxol-5-yl)-3-phenylpropionaldehyde

To a −78° C. solution of tert-butyl 3-(1,3-benzodioxol-5-yl)-3-phenylpropionate (Step 2) (1.36 mmol) in DCM (5 ml) was added diisobutylaluminium hydride (3 ml 1M solution, 3 mmol) dropwise and the resulting mixture stirred at −78° C. for 90 min. MeOH (3 ml) was added slowly and the mixture warmed to room temperature. Citric acid solution (10% aqueous, 5 ml) was added, the mixture stirred for 10 min. then filtered. The filtrate was dried and evaporated yielding the title compound which was used immediately in the next reaction.

The same sequence of three reactions was used to prepare 3-(4-chlorophenyl)-3-phenylpropionaldehyde, 3-(3,4-dichlorophenyl)-3-phenylpropionaldehyde, 3-(4-methoxyphenyl)-3-phenylpropionaldehyde, 3-(3-chlorophenyl)-3-phenylpropionaldehyde, 3-(4-methylphenyl)-3-phenylpropionaldehyde and 3-(4-trifluoromethylphenyl)-3-phenylpropionaldehyde.

### EXAMPLE 34

The ability of compounds to inhibit the binding of RANTES was assessed by an in vitro radioligand binding assay. Membranes were prepared from Chinese hamster ovary cells which expressed the recombinant human CCR5 receptor. These membranes were incubated with 0.1 nM iodinated RANTES, scintillation proximity beads and various concentrations of the compounds of the invention in 96-well plates. The amount of iodinated RANTES bound to the receptor was determined by scintillation counting. Competition curves were obtained for compounds and the concentration of compound which displaced 50% of bound iodinated RANTES was calculated (IC₅₀). Preferred compounds of formula (I) have an IC₅₀ of less than 50 μM.

### EXAMPLE 35

The ability of compounds to inhibit the binding of MIP-1α was assessed by an in vitro radioligand binding assay. Membranes were prepared from Chinese hamster ovary cells which expressed the recombinant human CCR5 receptor. These membranes were incubated with 0.1 nM iodinated MIP-1α, scintillation proximity beads and various concentrations of the compounds of the invention in 96-well plates. The amount of iodinated MlP-1α bound to the receptor was determined by scintillation counting. Competition curves were obtained for compounds and the concentration of compound which displaced 50% of bound iodinated MIP-1α was calculated (IC₅₀). Preferred compounds of formula (I) have an IC₅₀ of less than 50 μM.

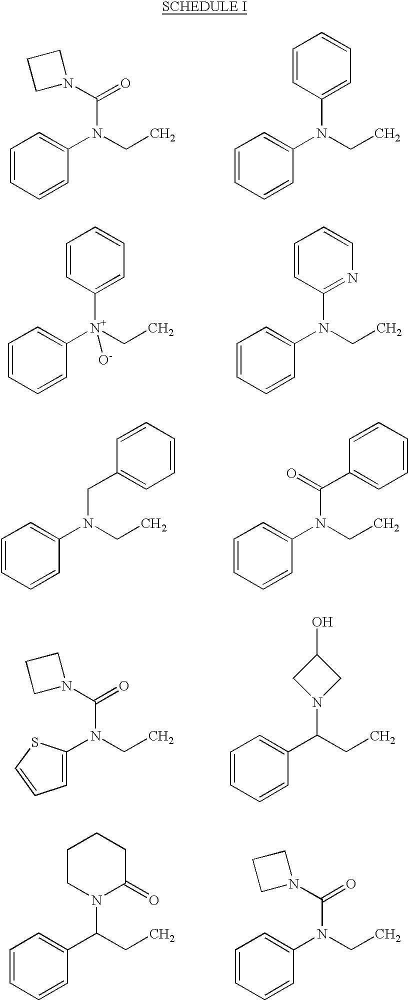

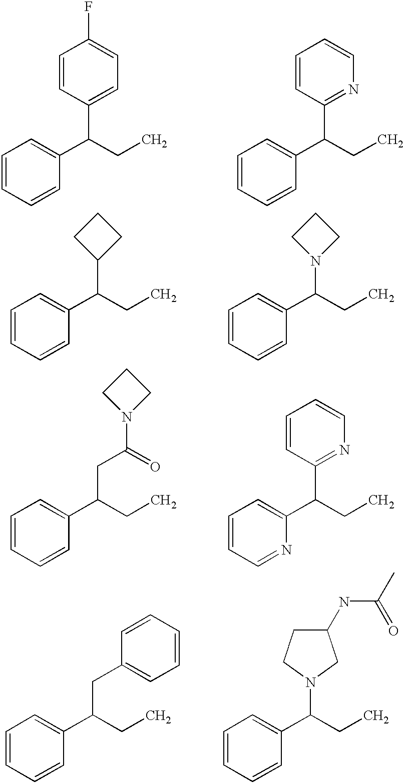

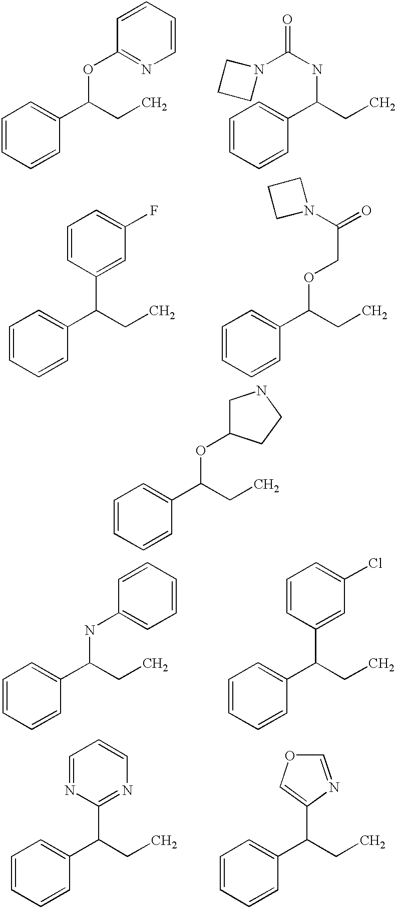

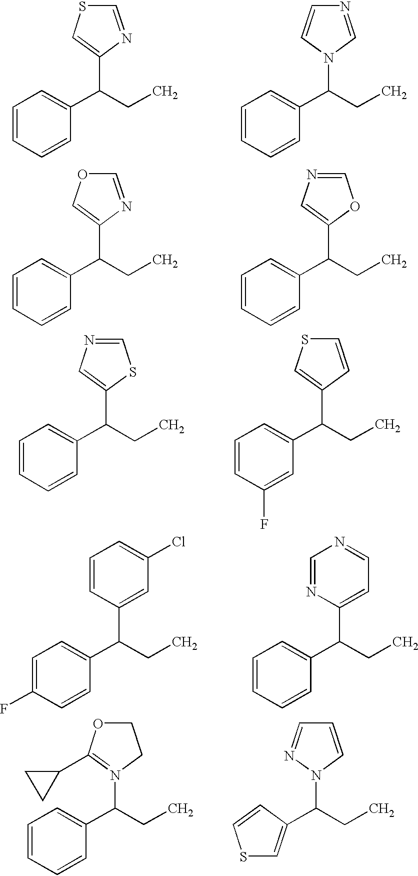

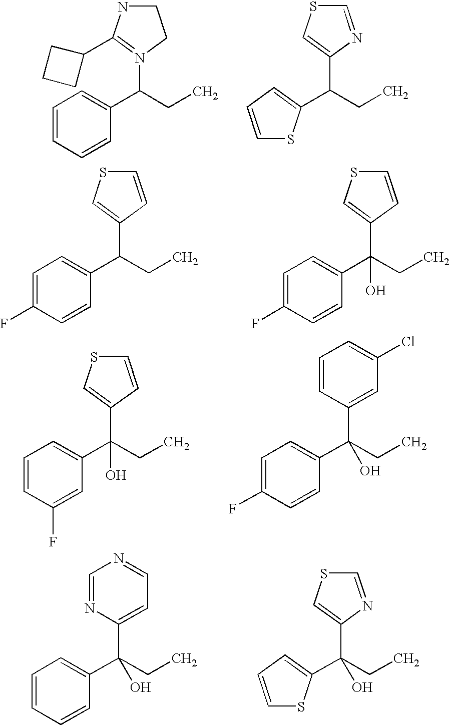

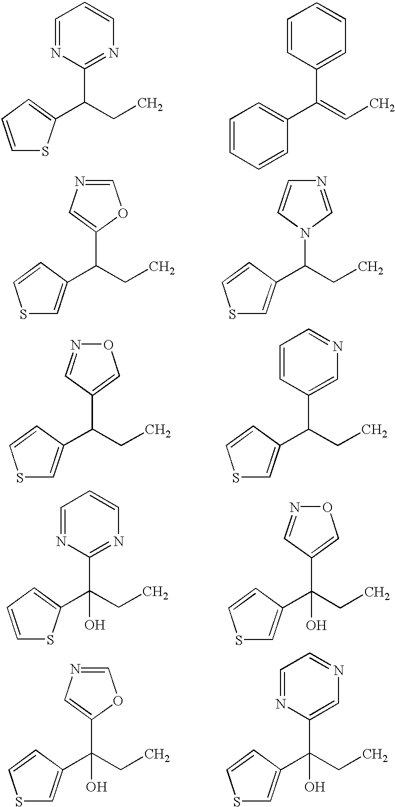

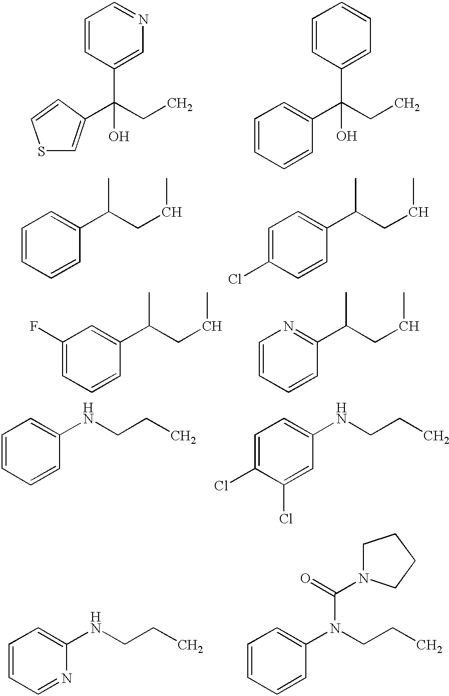

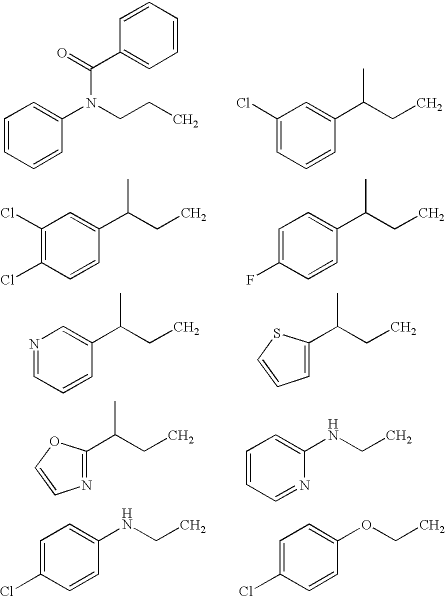

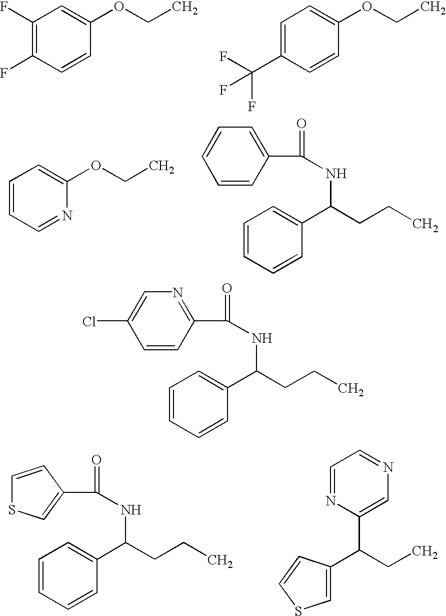

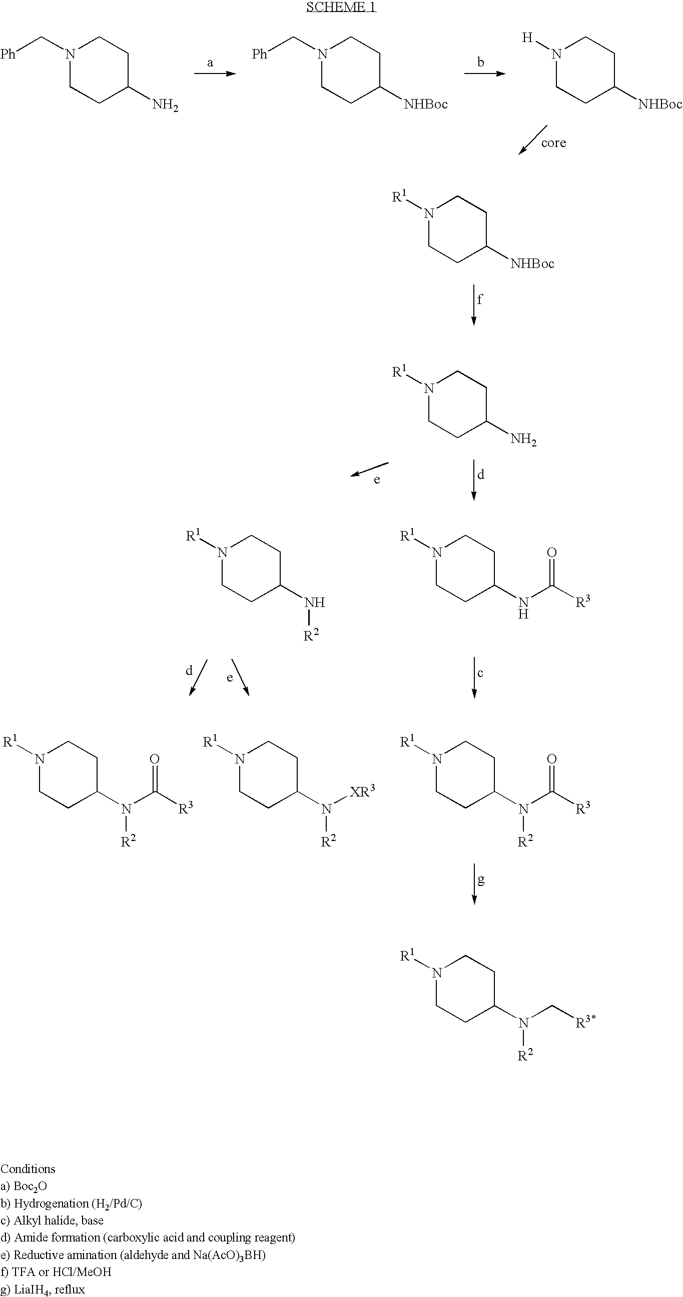

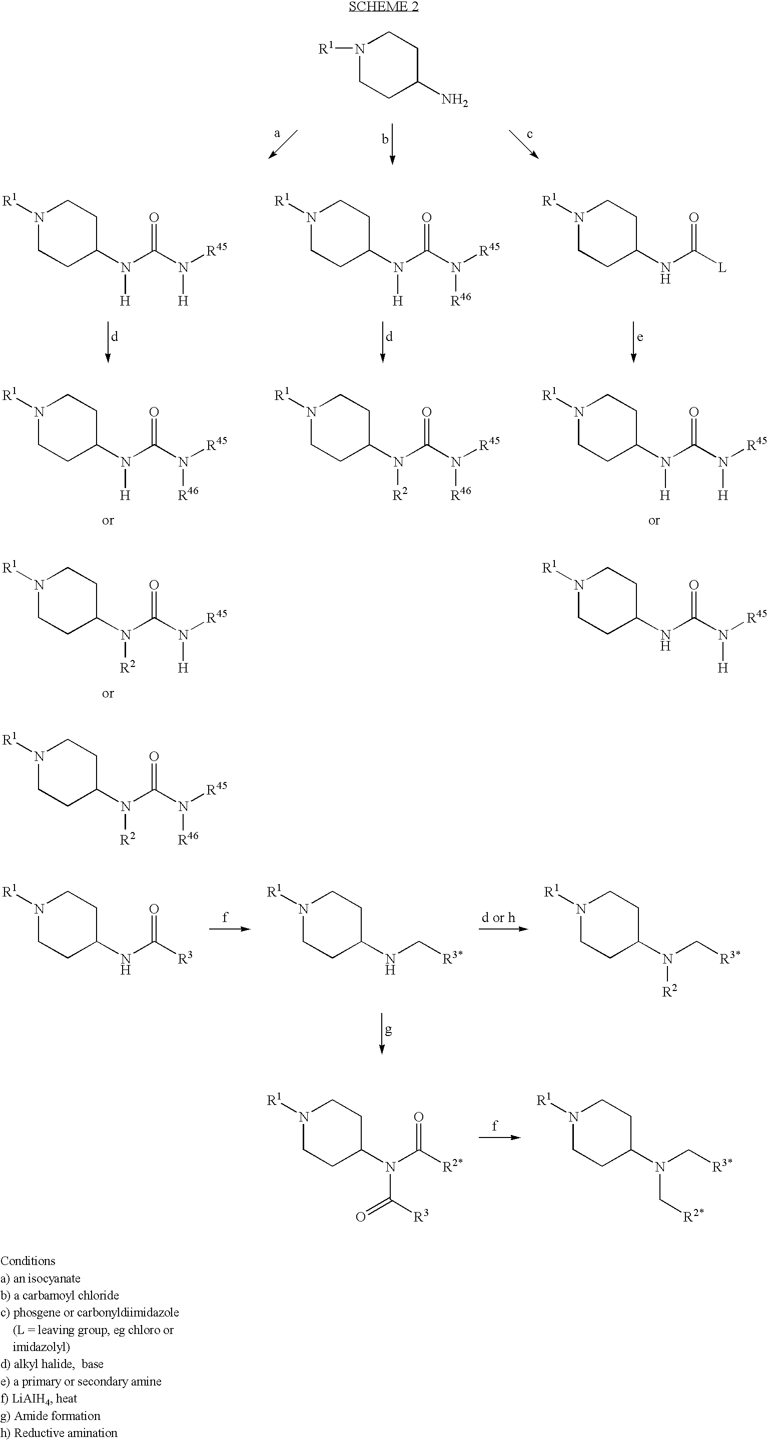

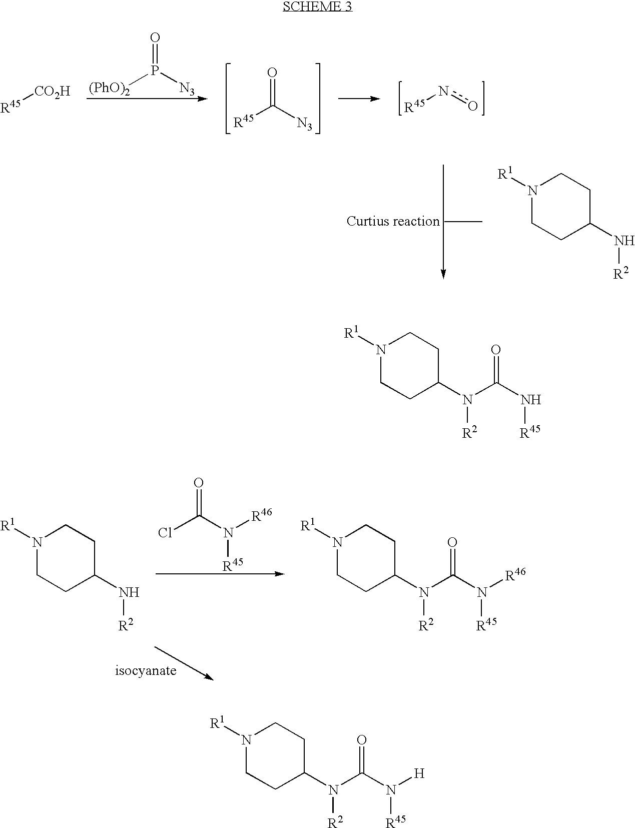

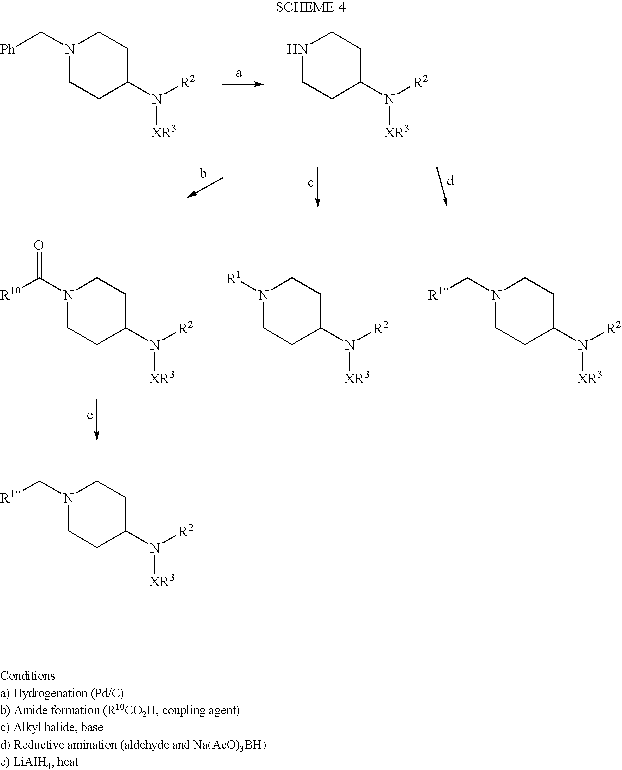

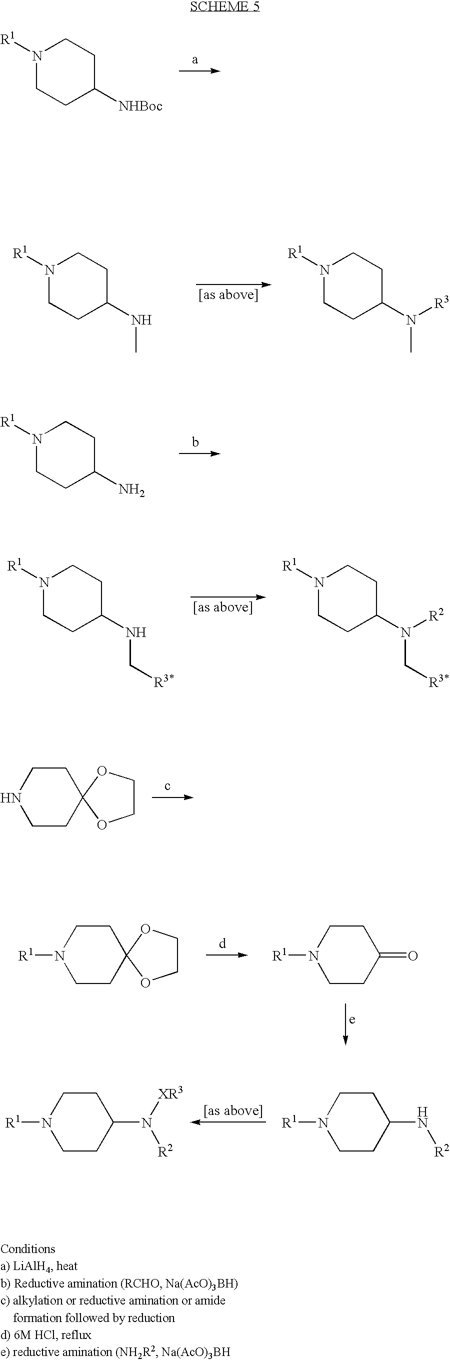

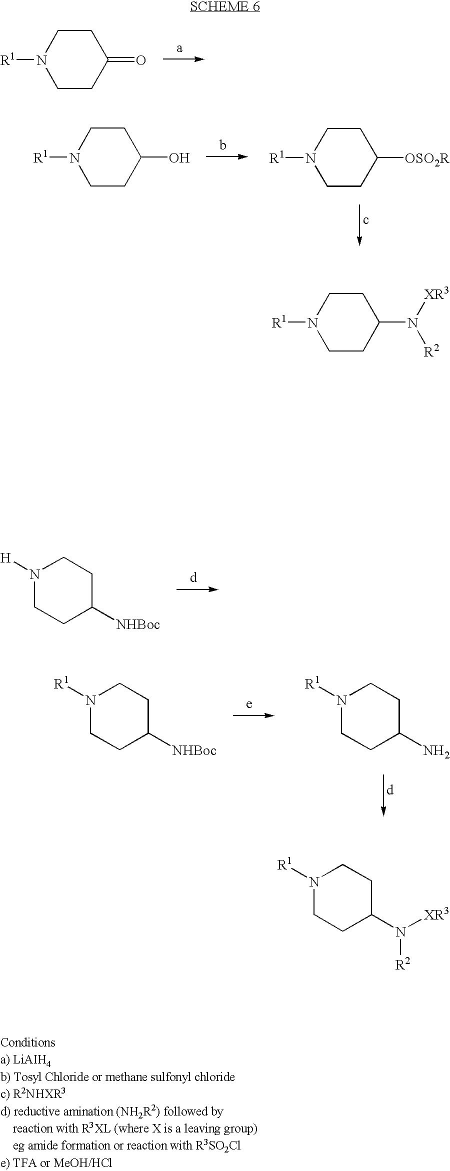

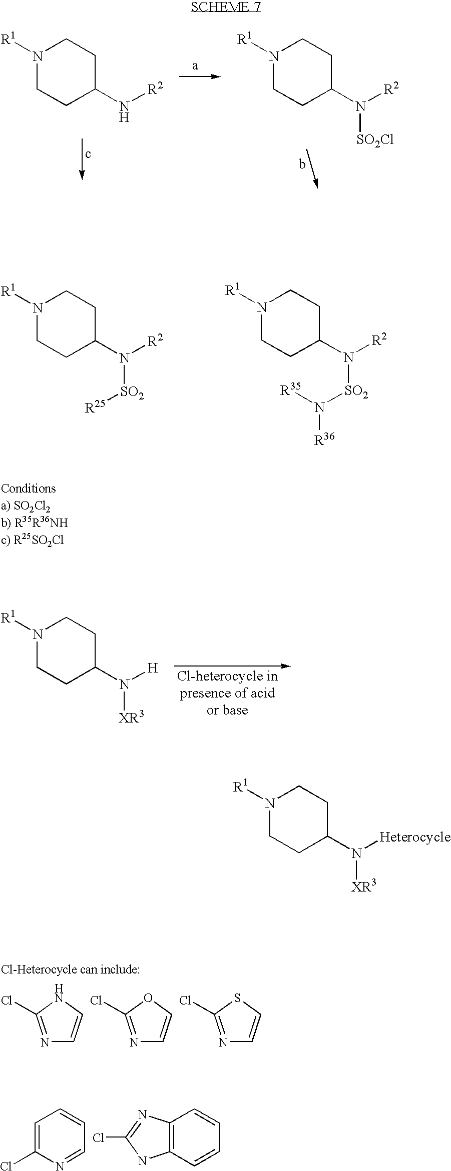

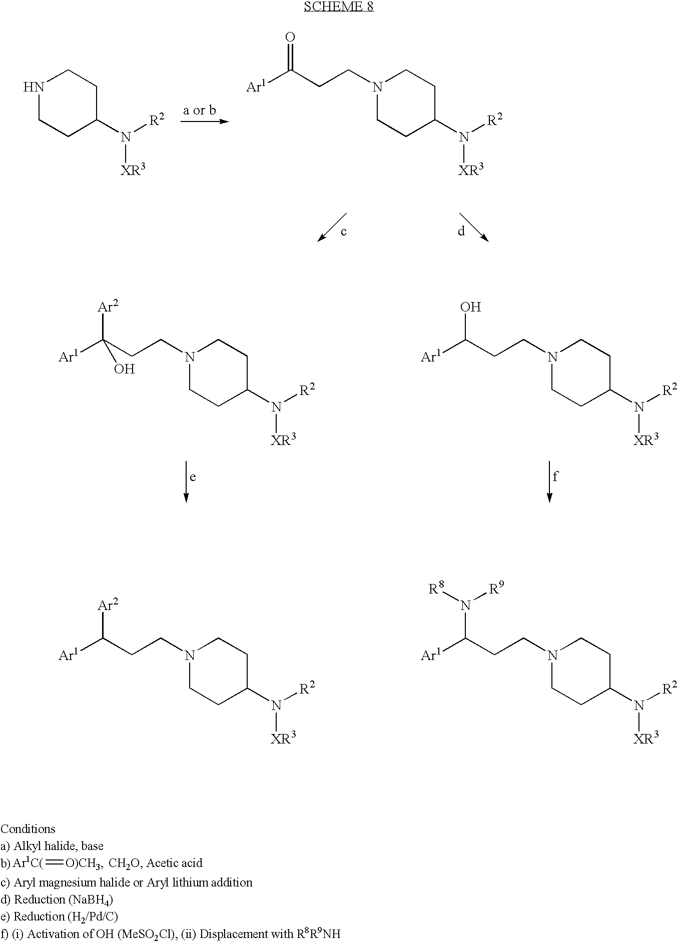

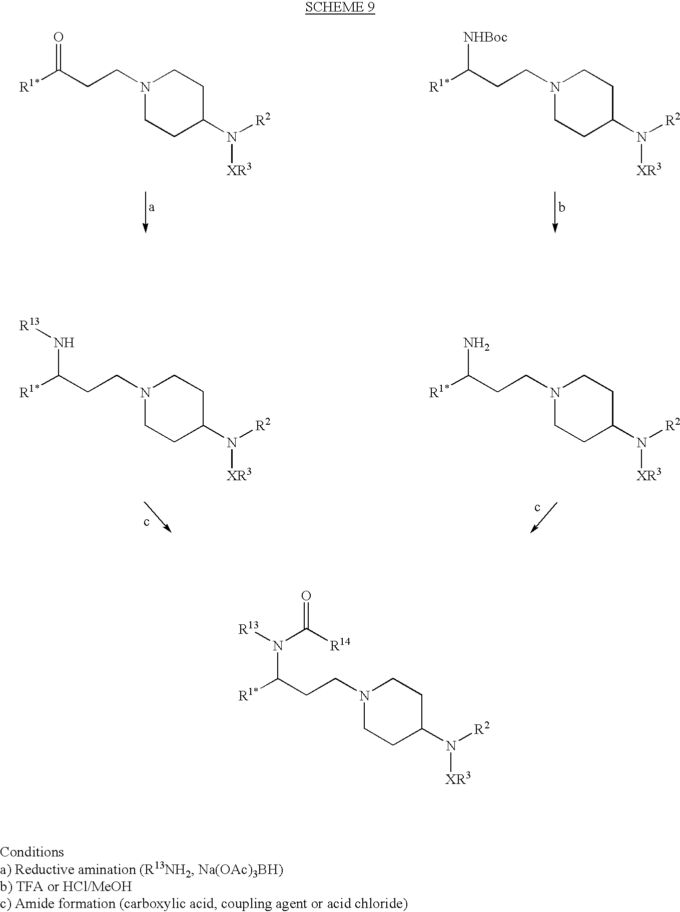

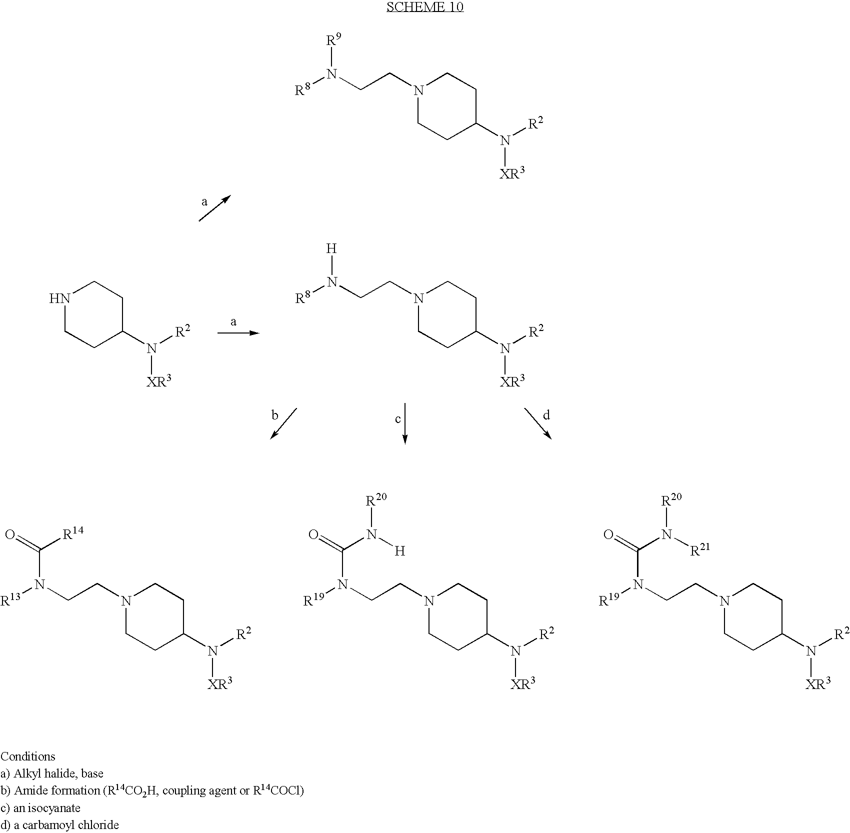

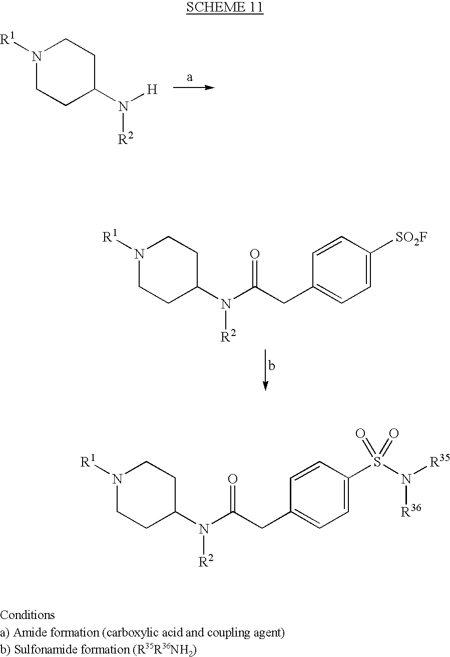

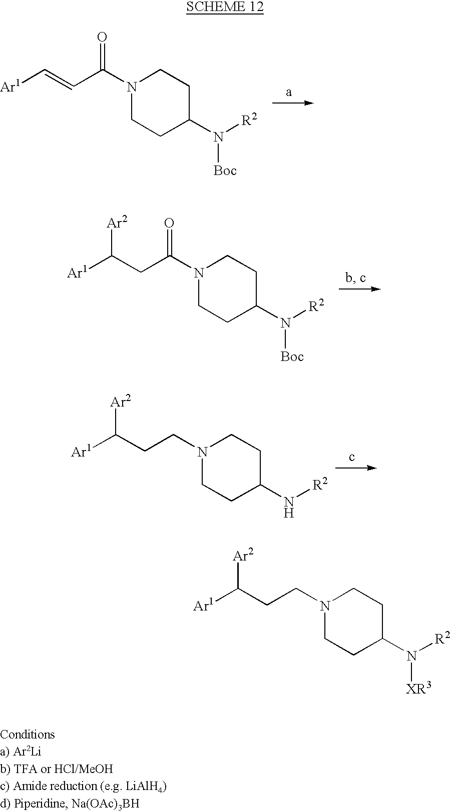

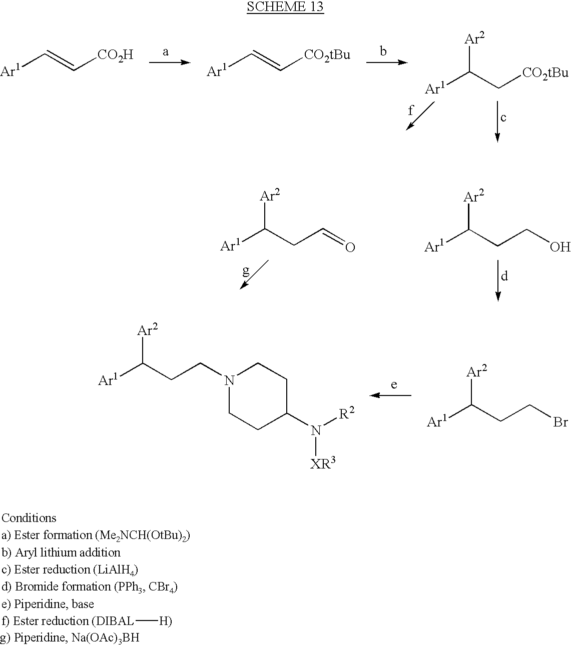

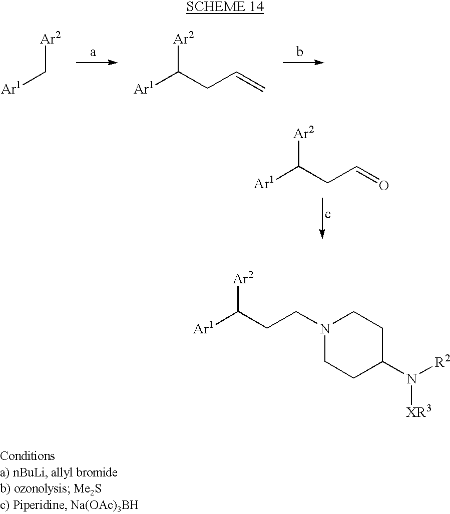

1. A compound of formula (I): 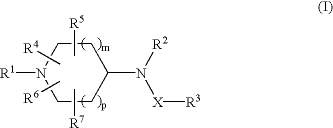
wherein:
R¹ is C₁₋₆ alkyl, C₃₋₇ cycloalkyl, C₃₋₈ alkenyl or C₃₋₈ alkynyl, each optionally substituted with one or more of: halo, hydroxy, cyano, nitro, C₃₋₇ cycloalkyl, NR⁸R⁹, C(O)R¹⁰, NR¹³C(O)R¹⁴, C(O)NR¹⁷R¹⁸, NR¹⁹C(O)NR²⁰R²¹, S(O)ₙR²², C₁₋₆ alkoxy (itself optionally substituted by heterocyclyl or C(O)NR²³R²⁴), heterocyclyl, heterocyclyloxy, aryl, aryloxy, heteroaryl or heteroaryloxy;
R² is hydrogen, C₁₋₈ alkyl, C₃₋₈ alkenyl, C₃₋₈ alkynyl, C₃₋₇ cycloalkyl, aryl, heteroaryl, heterocyclyl, aryl(C₁₋₄)alkyl, heteroaryl(C₁₋₄)alkyl or heterocyclyl(C₁₋₄)alkyl;
R³ is C₁₋₈ alkyl, C₂₋₈ alkenyl, NR⁴⁵R⁴⁶, C₂₋₈ alkynyl, C₃₋₇ cycloalkyl, C₃₋₇ cycloalkenyl, aryl, heteroaryl, heterocyclyl, aryl(C₁₋₄)alkyl, heteroaryl(C₁₋₄)alkyl or heterocyclyl(C₁₋₄)alkyl;
R⁴⁶ is C₁₋₈ alkyl, C₃₋₈ alkenyl, C₃₋₈ alkynyl, C₃₋₇ cycloalkyl, aryl, heteroaryl, heterocyclyl, aryl(C₁₋₄)alkyl, heteroaryl(C₁₋₄)alkyl or heterocyclyl(C₁₋₄)alkyl;
wherein the groups of R², R³ and R⁴⁶, and the heterocyclyl, aryl and heteroaryl moieties of R¹, are independently optionally substituted by one or more of halo, cyano, nitro, hydroxy, S(O)_{q}R²⁵, OC(O)NR²⁶R²⁷, NR²⁸R²⁹, NR³⁰C(O)R³¹, NR³²C(O)NR³³R³⁴, S(O)₂NR³⁵R³⁶, NR³⁷S(O)₂R³⁸, C(O)NR³⁹R⁴⁰, C(O)R⁴¹, CO₂R⁴², NR⁴³CO₂R⁴⁴, C₁₋₆ alkyl, C₃₋₁₀ cycloalkyl, C₁₋₆ haloalkyl, C₁₋₆ alkoxy, C₁₋₆ haloalkoxy, phenyl, phenyl(C₁₋₄)alkyl, phenoxy, phenylthio, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halo, hydroxy, nitro, S(O)ₖC₁₋₄ alkyl, S(O)₂NH₂, cyano, C₁₋₄ alkyl, C₁₋₄ alkoxy, C(O)NH₂, C(O)NH(C₁₋₄ alkyl), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; the C₃₋₇ cycloalkyl, aryl, heteroaryl and heterocyclyl moieties of R¹, R² and R³ being additionally optionally substituted with C₁₋₆ alkyl, C₂₋₆ alkenyl, C₂₋₆ alkynyl or C₁₋₆ alkoxy(C₁₋₆)alkyl;
R⁴, R⁵, R⁶ and R⁷ are, independently, hydrogen, C₁₋₆ alkyl {optionally substituted by halo, cyano, hydroxy, C₁₋₄ alkoxy, OCF₃, NH₂, NH(C₁₋₄ alkyl), N(C₁₋₄ alkyl)₂, NHC(O)(C₁₋₄ alkyl), N(C₁₋₄ alkyl)C(O)(C₁₋₄ alkyl), NHS(O)₂(C₁₋₄ alkyl), N(C₁₋₄ alkyl)S(O)₂(C₁₋₄ alkyl), CO₂(C₁₋₄ alkyl), C(O)NH(C₁₋₄ alkyl), C(O)N(C₁₋₄ alkyl)₂, C(O)NH₂, CO₂H, S(O)₂(C₁₋₄ alkyl), S(O)₂NH(C₁₋₄ alkyl), S(O)₂N(C₁₋₄ alkyl)₂, heterocyclyl or C(O)(heterocyclyl)}, S(O)₂NH₂, S(O)₂NH(C₁₋₄ alkyl), C(O)N(C₁₋₄ alkyl)₂, C(O)(C₁₋₄ alkyl), CO₂H, CO₂(C₁₋₄ alkyl) or C(O)(heterocyclyl); or two of R⁴, R⁵, R⁶ and R⁷ can join to form, together with the ring to which they are attached, a bicyclic ring system; or two of R⁴, R⁵, R⁶ and R⁷ can form an endocyclic bond (thereby resulting in an unsaturated ring system);
X is C(O), S(O)₂, C(O)C(O), a direct bond or C(O)C(O)NR⁴⁷;
k, m, n, p and q are, independently, 0, 1 or 2;
R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³ and R⁴⁴ are, independently, C₁₋₈ alkyl, C₃₋₈ alkenyl, C₃₋₈ alkynyl, C₃₋₇ cycloalkyl, aryl, heteroaryl or heterocyclyl each or which is optionally substituted by halo, cyano, nitro, hydroxy, C₁₋₄ alkyl, C₄ alkoxy, SCH₃, S(O)CH₃, S(O)₂CH₃, NH₂, NHCH₃, N(CH₃)₂, NHC(O)NH₂, C(O)NH₂, NHC(O)CH₃, S(O)₂N(CH₃)₂, S(O)₂NHCH₃, CF₃, CHF₂, CH₂F, CH₂CF₃ or OCF₃; and R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³ and R⁴⁴ may additionally be hydrogen;
R⁸, R⁹, R¹⁰, R¹³, R¹⁴, R¹⁷, R¹⁸, R¹⁹, R²⁰, R²¹, R²³, R²⁴, R⁴⁵ and R⁴⁷ are, independently, hydrogen, alkyl {optionally substituted by halo, hydroxy, C₁₋₆ alkoxy, C₁₋₆ haloalkoxy, heterocyclyl or phenyl (itself optionally substituted by halo, hydroxy, cyano, C₁₋₄ alkyl or C₁₋₄ alkoxy)}, phenyl (itself optionally substituted by halo, hydroxy, nitro, S(O)ₖC₁₋₄ alkyl, S(O)₂NH₂, cyano, C₁₋₄ alkyl, C₁₋₄ alkoxy, C(O)NH₂, C(O)NH(C₁₋₄ alkyl), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃) or heteroaryl (itself optionally substituted by halo, hydroxy, nitro, S(O)ₖC₁₋₄ alkyl, S(O)₂NH₂, cyano, C₁₋₄ alkyl, C₁₋₄ alkoxy, C(O)NH₂, C(O)NH(C₁₋₄ alkyl), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃);
R²² is alkyl {optionally substituted by halo, hydroxy, C₁₋₆ alkoxy, C₁₋₆ haloalkoxy, heterocyclyl or phenyl (itself optionally substituted by halo, hydroxy, cyano, C₁₋₄ alkyl or C₁₋₄ alkoxy)}, phenyl (itself optionally substituted by halo, hydroxy, cyano, C₁₋₄ alkyl or C₁₋₄ alkoxy) or heteroaryl (itself optionally substituted by halo, hydroxy, cyano, C₁₋₄ alkyl or C₁₋₄ alkoxy);
the pairs of substituents: R⁸ and R⁹, R¹³ and R¹⁴, R¹⁷ and R¹⁸, R²⁰ and R²¹, R²³ and R²⁴, R²⁶ and R²⁷, R²⁸ and R²⁹, R³⁰ and R³¹, R³² with either R³³ or R³⁴, R³³ and R³⁴, R³⁵ and R³⁶, R³⁷ and R³⁸, R³⁹ and R⁴⁰ and R⁴³ and R⁴⁴ may, independently, join to form a ring and such a ring may also comprise an oxygen, sulphur or nitrogen atom;
where for any of the foregoing heterocyclic groups having a ring —N(H)— moiety, that —N(H)— moiety may be optionally substituted by C₁₋₄ alkyl (itself optionally substituted by hydroxy), C(O)(C₁₋₄ alkyl), C(O)NH(C₁₋₄ alkyl), C(O)N(C₁₋₄ alkyl)₂ or S(O)₂(C₁₋₄ alkyl);
a ring nitrogen and/or sulphur atom is optionally oxidised to form an N-oxide and/or an S-oxide;
foregoing heteroaryl or heterocyclyl rings are C- or, where possible, N-linked;
or a pharmaceutically acceptable salt thereof or a solvate thereof.

2. A compound as claimed in claim 1 wherein heteroaryl is pyrrolyl, imidazolyl, pyrazolyl, 1,2,3-triazolyl, 1,2,4-triazolyl, oxazolyl, isoxazolyl, thiazolyl, isothiazolyl, pyridinyl, pyrimidinyl, pyrazinyl, pyridazinyl, thienyl, furyl, quinolinyl, isoquinolinyl, dihydroisoquinolinyl, indolyl, benzimidazolyl, benzo[b]furyl, benzo[b]thienyl, phthalazinyl, indanyl, oxadiazolyl or benzthiazolyl.

3. A compouind as claimed in claim 1 or 2 wherein aryl is phenyl.

4. A compound as claimed in claim 1, 2 or 3 wherein heterocyclyl is piperidinyl, morpholinyl, pyrrolidinyl, piperazinyl or tetrahydrofuryl.

5. A compound as claimed in claim 1, 2, 3 or 4 wherein R⁴, R⁵, R⁶ and R⁷ are all hydrogen.

6. A compound as claimed in claim 1, 2, 3, 4, or 5 wherein X is C(O).

7. A compound as claimed in claim 1, 2, 3, 4, 5 or 6 wherein m and p are both 1.

8. A compound as claimed in claim 1, 2, 3, 4, 5, 6 or 7 wherein R² is methyl, ethyl, allyl, cyclopropyl or propargyl.

9. A compound as claimed in claim 1, 2, 3, 4, 5, 6, 7 or 8 wherein R³ is NR⁴⁵R⁴⁶, aryl, heteroaryl, aryl(C₁₋₄)alkyl or heteroaryl(C₁₋₄)alkyl; R⁴⁵ is hydrogen or C₁₋₆ alkyl; R⁴⁶ is aryl, heteroaryl, aryl(C₁₋₄)alkyl or heteroaryl(C₁₋₄)alkyl; wherein the aryl and heteroaryl groups of R³ and R⁴⁶ are independently substituted by S(O)_{q}R²⁵, OC(O)NR²⁶R²⁷, NR³²C(O)NR³³R³⁴ or C(O)R⁴¹, and optionally further substituted by one or more of halo, cyano, nitro, hydroxy, C₁₋₆ alkyl, C₂₋₆ alkenyl, C₂₋₆ alkynyl, C₁₋₆ alkoxy(C₁₋₆)alkyl, S(O)_{q}R²⁵, OC(O)NR²⁶R²⁷, NR²⁸R²⁹, NR³⁰C(O)R³¹, NR³²C(O)NR³³R³⁴, S(O)₂NR³⁵R³⁶, NR³⁷S(O)₂R³⁸, C(O)NR³⁹R⁴⁰, C(O)R⁴¹, CO₂R⁴², NR⁴³CO₂R⁴⁴, C₃₋₁₀ cycloalkyl, C₁₋₆ haloalkyl, C₁₋₆ alkoxy, C₁₋₆ haloalkoxy, phenyl, phenyl(C₁₋₄)alkyl, phenoxy, phenylthio, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halo, hydroxy, nitro, S(O)ₖC₁₋₄ alkyl, S(O)₂NH₂, cyano, C₁₋₄ alkyl, C₁₋₄ alkoxy, C(O)NH₂, C(O)NH(C₁₋₄ alkyl), CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; wherein q, k, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³ and R⁴⁴ are as defined in claim 1.

10. A compound as claimed in claim 1, 2, 3, 4, 5, 6, 7, 8 or 9 wherein R¹ is 2,6-dimethoxybenzyl, 2,4,6-trimethoxybenzyl, 2,4-dimethoxy-6-hydroxybenzyl, 3-(4-dimethylamino-phenyl)prop-2-enyl, (1-phenyl-2,5-dimethylpyrrol-3-yl)methyl, 2-phenylethyl, 3-phenylpropyl, 3-R/S-phenylbutyl, 3-cyano-3,3-diphenylpropyl, 3-cyano-3-phenylpropyl, 4-(N-methylbenzamido)-3-phenylbutyl or 3,3-diphenylpropyl.

11. A pharmaceutical composition which comprises a compound of the formula (I) as claimed in claim 1, or a pharmaceutically acceptable salt thereof or solvate thereof, and a pharmaceutically acceptable adjuvant, diluent or carrier.

12. A compound of the formula (I), or a pharmaceutically acceptable salt thereof or solvate thereof, for use as a medicament.

13. A compound of formula (I), or a pharmaceutically acceptable salt thereof or solvate thereof, in the manufacture of a medicament for use in therapy.

14. A compound of formula (I), or a pharmaceutically acceptable salt thereof or solvate thereof, in the manufacture of a medicament for use in modulating CCR5 receptor activity in a warm blooded animal.

15. A method of treating a patient comprising administering a compound of formula (I) as claimed in claim 1, or a pharmaceutically acceptable salt thereof or solvate thereof, or a composition as claimed in claim 11.

16. A process for the preparation of a compound of formula (I) as claimed in claim 1 comprising:
a. reductively aminating a compound of formula (II): 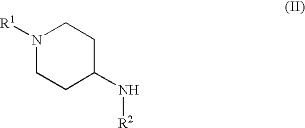
with an aldehyde R³CHO; or
b. where R¹ is optionally substituted alkyl, reacting a compound of formula (III): 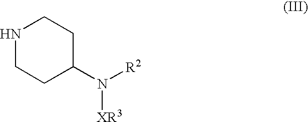
with an alkyl halide, in the presence of a base.
